SEQUENCE LISTING (40 sequences)

SEQ ID NO: 1 - protein, 403 aa, Homo sapiens [sequence omitted]

SEQ ID NO: 2 - protein, 9 aa, Homo sapiens
ALLEIASCL

SEQ ID NO: 3 - protein, 21 aa, Homo sapiens
DTLLKALLEIASCLEKALQVF

SEQ ID NO: 4 - protein, 9 aa, Homo sapiens
QLRERVEKL

SEQ ID NO: 5 - protein, 9 aa, Homo sapiens
FLVSLLVEI

SEQ ID NO: 6 - protein, 9 aa, Homo sapiens
TLLKALLEI

SEQ ID NO: 7 - protein, 9 aa, Homo sapiens
FIAKHLPDL

SEQ ID NO: 8 - protein, 9 aa, Homo sapiens
VLSKGDAGL

SEQ ID NO: 9 - protein, 9 aa, Homo sapiens
DLMNFLKTV

SEQ ID NO: 10 - protein, 9 aa, Homo sapiens
VLLGIQQTA

SEQ ID NO: 11 - protein, 9 aa, Homo sapiens
KVLPRNIAV

SEQ ID NO: 12 - protein, 10 aa, Homo sapiens
KLNMLSIDHL

SEQ ID NO: 13 - protein, 10 aa, Homo sapiens
SLRSYHLQIV

SEQ ID NO: 14 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 15 - protein, 9 aa, Homo sapiens
LLNAFTVTV

SEQ ID NO: 16 - protein, 9 aa, Homo sapiens
ILLCLGVAL

SEQ ID NO: 17 - protein, 9 aa, Homo sapiens
ILGAILLCL

SEQ ID NO: 18 - protein, 9 aa, Homo sapiens
ALQITDVKL

SEQ ID NO: 19 - protein, 9 aa, Homo sapiens
KLFNVTSTL

SEQ ID NO: 20 - protein, 9 aa, Homo sapiens
RLLKDQLSL

SEQ ID NO: 21 - protein, 9 aa, Homo sapiens
QLSLGNAAL

SEQ ID NO: 22 - protein, 9 aa, Homo sapiens
KINQRILVV

SEQ ID NO: 23 - protein, 9 aa, Homo sapiens
HLVILGAIL

SEQ ID NO: 24 - protein, 9 aa, Homo sapiens
RINTTTNEI

SEQ ID NO: 25 - protein, 9 aa, Homo sapiens
CLGVALTFI

SEQ ID NO: 26 - protein, 9 aa, Homo sapiens
QLDLAALIV

SEQ ID NO: 27 - protein, 9 aa, Homo sapiens
SLGNAALQI

SEQ ID NO: 28 - protein, 10 aa, Homo sapiens
VILGAILLCL

SEQ ID NO: 29 - protein, 10 aa, Homo sapiens
HTAELVIPEL

SEQ ID NO: 30 - protein, 10 aa, Homo sapiens
FIFMTYWHLL

SEQ ID NO: 31 - protein, 10 aa, Homo sapiens
VIWTSSDHQV

SEQ ID NO: 32 - protein, 19 aa, Homo sapiens
FMTYWHLLNAFTVTVPKDL

SEQ ID NO: 33 - RNA, 25 nt, Artificial Sequence
  PD-L1 siRNA duplex sense sequence
ccuacuggcauuugcugaacgcauu

SEQ ID NO: 34 - RNA, 25 nt, Artificial Sequence
  PD-L1 siRNA duplex antisense sequence
aaugcguucagcaaaugccaguagg

SEQ ID NO: 35 - DNA, 21 bp, Artificial Sequence
  IDO siRNA duplex - siRNA1 sense sequence
acaucugccugaucucauatt

SEQ ID NO: 36 - DNA, 21 bp, Artificial Sequence
  IDO siRNA duplex - siRNA1 antisense sequence
uaugagaucaggcagaugutt

SEQ ID NO: 37 - DNA, 21 bp, Artificial Sequence
  IDO siRNA duplex - siRNA2 sense sequence
ccacgaucaugugaacccatt

SEQ ID NO: 38 - DNA, 21 bp, Artificial Sequence
  IDO siRNA duplex - siRNA2 antisense sequence
uggguucacaugaucguggat

SEQ ID NO: 39 - DNA, 21 bp, Artificial Sequence
  IDO siRNA duplex - siRNA3 sense sequence
cgaucaugugaacccaaaatt

SEQ ID NO: 40 - DNA, 21 bp, Artificial Sequence
  IDO siRNA duplex - siRNA3 antisense sequence
uuuuggguucacaugaucgtg

(19) United States
(12) Patent Application Publication
(10) Pub. No.: US 2024/0307516 A1
(43) Pub. Date: Sep. 19, 2024

(54) COMBINATION THERAPY FOR CANCER

(71) Applicant: IO BIOTECH APS, Copeenhagen (DK)

(72) Inventor: Mads Hald ANDERSEN, Naerum (DK)

(21) Appl. No.: 18/550,854

(22) PCT Filed: Aug. 31, 2021
(86) PCT No.: PCT/EP2021/074064

§ 371 Date: Sep. 15, 2023

(30) Foreign Application Priority Data
Mar. 17, 2021 (GB) 2103673.6

(51) Int. Cl. A61K 39/00; A61K 31/405; A61K 31/407; A61K 31/4245; A61K 31/517; A61P 35/00; C07K 16/28; G01N 33/569

(52) U.S. Cl. CPC A61K 39/001154; A61K 31/405; A61K 31/407; A61K 31/4245; A61K 31/517; A61K 39/001111; A61P 35/00; C07K 16/2818; C12Y 113/11052; G01N 33/56972; A61K 2039/505; A61K 2039/55516

(57) ABSTRACT

The present disclosure relates to the field of cancer treatment and prevention. The present disclosure further relates to (a) a first immune checkpoint polypeptide or a polynucleotide encoding the same; (b) a second immune checkpoint polypeptide or a polynucleotide encoding the same; and (c) an immune checkpoint inhibitor. The disclosure also relates to compositions comprising one or more of (a), (b) and/or (c), methods of use, and kits comprising same. The disclosure also relates to methods for stratifying cancer patients and methods for monitoring the treatment response.

### CROSS-REFERENCE TO RELATED APPLICATIONS

This application claims priority to EP 2103673.6, filed Mar. 17, 2016, and which is incorporated herein by reference in its entirety.

### FIELD

The present disclosure relates to the field of cancer treatment and prevention. The present disclosure further relates to (a) a first immune checkpoint polypeptide or a polynucleotide encoding the same; (b) a second immune checkpoint polypeptide or a polynucleotide encoding the same; and (c) an immune checkpoint inhibitor. The disclosure also relates to compositions comprising one or more of (a), (b) and/or (c), methods of use, and kits comprising same. The present disclosure also relates to methods for stratifying cancer patients and methods for monitoring treatment response.

### BACKGROUND

The human immune system is capable of mounting a response against cancerous tumours. Exploiting this response is increasingly seen as one of the most promising routes to treat or prevent cancer. The key effector cell of a long-lasting anti-tumour immune response is the activated tumour-specific effector T cell. However, although cancer patients usually have T cells specific for tumour antigens, the activity of these T cells is frequently suppressed by inhibitory factors and pathways, and cancer remains a leading cause of premature deaths in the developed world.

Over the past decade treatments have emerged which specifically target immune system checkpoints. An example of this is Ipilimumab, which is a fully human IgG1 antibody specific for CTLA-4. Treatment of metastatic melanoma with Ipilimumab was associated with an overall response rate of 10.9% and a clinical benefit rate of nearly 30% in a large phase III study and subsequent analyses have indicated that responses may be durable and long lasting. However, these figures still indicate that a majority of the patients do not benefit from treatment, leaving room for improvement.

Accordingly, there exists a need for methods for the prevention or treatment of cancer which augment the T cell anti-tumour response in a greater proportion of patients, but without provoking undesirable effects such as autoimmune disease.

### SUMMARY

Despite remarkable advances in the treatment of cancer with immune checkpoint inhibitors (ICIs), including inhibitors programmed death-1 (PD-1) and cytotoxic T lymphocyte antigen-4 (CTLA-4), substantial percentages of patients are resistant, or develop resistance to, ICI monotherapy (See e.g., Robert, C. et al., Lancet Oncol. 20, 1239-1251 (2019)). The combination of anti-CTLA-4 (αCTLA-4) and anti-PD-1 (αPD-1) is to date the most effective therapy resulting in a response rate of around 60% in metastatic melanoma; however, 50% of the patients also develop severe adverse events (Weber et al, Oncologist 1-11 (2016) doi:10.1634/theoncologist. 2016-0055; Larkin, J. et al. N. Engl. J. Med. 381, 1535-1546 (2019). Therefore, there remains a need for an equally effective but less toxic treatment.

The antitumor activity of αPD1 monotherapy may be compromised by a limited pool of pre-existing naive and primed tumour-specific T-cells. Immune modulatory vaccines targeting tumoral immune escape mechanisms offer a treatment strategy that is applicable to general patient populations, as these escape mechanisms are found in numerous cancer types and across diverse patient populations. This is in contrast to patient-specific-neoantigen cancer vaccines, which are specifically tailored to a particular tumour and are not broadly applicable (Ott, P. A. et al. Cell 740 183, 347-362.e24 (2020); Andersen, M. H. Semin. Immunopathol. 41, 1-3 (2019)).

Circulating cytotoxic T-cells against two immune checkpoint molecules, indoleamine 2,3-dioxygenase (IDO) and programmed death-ligand 1 (PD-L1), have been detected in the blood of cancer patients and, to a lesser extent, healthy donors (Munir, S. et al., Oncoimmunology 2, e23991 (2013); Ahmad, S. et al., Cancer Immunol. Immunother. 65, 797-804 (2016); Andersen, M. Cancer Immunol. Immunother. 61, 1289-1297 (2012); Sorensen, Cancer Res. 71, 2038-2044 (2011); Ahmad, S., et al., Leukemia 28, 236-8 (2014); Andersen, M. H. Oncoimmunology 1, 1211-1212 (2012)). These T-cells directly recognize tumour cells as well as immunosuppressive cells in the tumour microenvironment and can therefore be exploited to restrict the range of immunosuppressive signals and reverse the immunosuppressive nature of the tumour microenvironment. Thus, the IDO/PD-L1 immune-modulating vaccine strategy described herein may lead to a translatable strategy for improving the efficacy of αPD1 therapy through activation of these specific T-cells.

In some embodiments, the present disclosure provides a method for treating a cancer in a subject in need thereof comprising administering to the subject: a first immune checkpoint polypeptide or a polynucleotide encoding the same; a second immune checkpoint polypeptide or a polynucleotide encoding the same; and an immune checkpoint inhibitor.

In some embodiments, the present disclosure provides a method of preventing disease progression in a subject suffering from a cancer comprising administering to the subject: a first immune checkpoint polypeptide or a polynucleotide encoding the same; a second immune checkpoint polypeptide or a polynucleotide encoding the same; and an immune checkpoint inhibitor.

In some embodiments, the present disclosure provides a method of reducing tumor volume in a subject suffering from a cancer comprising administering to the subject: a first immune checkpoint polypeptide or a polynucleotide encoding the same; a second immune checkpoint polypeptide or a polynucleotide encoding the same; and an immune checkpoint inhibitor.

In some embodiments, the subject has not previously received treatment with the immune checkpoint inhibitor. In some embodiments, the subject has previously received treatment with the immune checkpoint inhibitor. In some embodiments, the subject was refractory to the treatment with the immune checkpoint inhibitor or developed resistance to the immune checkpoint inhibitor during the course of the previous treatment.

In some embodiments, the first and second immune checkpoint polypeptide are independently selected from an IDO1 peptide, a PD-1 peptide, a PD-L1 peptide, a PD-L2 peptide, a CTLA4 peptide, a B7-H3 peptide, a B7-H4 peptide, an HVEM peptide, a BTLA peptide, a GAL9 peptide, a TIM3 peptide, a LAG3 peptide, or a KIR polypeptide.

In some embodiments, the first immune checkpoint polypeptide is an IDO1 polypeptide and wherein the second immune checkpoint polypeptide is a PD-L1 polypeptide. In some embodiments, the IDO1 polypeptide consists of up to 50 consecutive amino acids of SEQ ID NO: 1, and wherein said consecutive amino acids comprise the sequence of ALLEIASCL (SEQ ID NO: 2) or the sequence of DTLLKALLEIASCLEKALQVF (SEQ ID NO: 3). In some embodiments, the IDO1 polypeptide comprises or consists of the sequence of ALLEIASCL (SEQ ID NO: 2) or the sequence of DTLLKALLEIASCLEKALQVF (SEQ ID NO: 3). In some embodiments, the PD-L1 polypeptide consists of up to 50 consecutive amino acids of SEQ ID NO: 14, and wherein said consecutive amino acids comprise the sequence of any one of SEQ ID NOs: 15 to 32. In some embodiments, the PD-L1 polypeptide consists of up to 50 consecutive amino acids of SEQ ID NO: 14, and wherein said consecutive amino acids comprise the sequence of any one of SEQ ID NOs: 15, 25, 28 or 32. In some embodiments, the PD-L1 peptide comprises or consists of the sequence of FMTYWHLLNAFTVTVPKDL (SEQ ID NO: 32).

In some embodiments, the IDO1 polypeptide comprises or consists of the sequence of ALLEIASCL (SEQ ID NO: 2) or the sequence of DTLLKALLEIASCLEKALQVF (SEQ ID NO: 3) and the PD-L1 peptide comprises or consists of the sequence of FMTYWHLLNAFTVTVPKDL (SEQ ID NO: 32).

In some embodiments, the immune checkpoint inhibitor is an antibody or small molecule inhibitor (SMI). In some embodiments, the SMI is an inhibitor of IDO1. In some embodiments, the SMI is selected from Epacadostat (INCB24360), Indoximod, GDC-0919 (NLG919), and F001287. In some embodiments, the antibody binds to CTLA4 or PD1. In some embodiments, the antibody that binds to CTLA4 is ipilimumab. In some embodiments, the antibody that binds to PD-1 is pembrolizumab or nivolumab.

In some embodiments, the first and second immune checkpoint polypeptides or polynucleotides encoding the same are administered as a first composition and the immune checkpoint inhibitor is administered as a second composition. In some embodiments, the first and second immune checkpoint polypeptides or polynucleotides encoding the same and the immune checkpoint inhibitor are administered as one composition.

In some embodiments, the compositions further comprise an adjuvant or carrier. In some embodiments, the adjuvant is selected from herein said adjuvant is a Montanide ISA adjuvant, a bacterial DNA adjuvant, an oil/surfactant adjuvant, a viral dsRNA adjuvant, an imidazoquinoline, and GM-CSF. In some embodiments, the Montanide ISA adjuvant is selected from Montanide ISA 51 and Montanide ISA 720.

In some embodiments, the disease does not progress for at least 12 months, at least 2 years, at least 3 years, at least 4 years, at least 5 years, or longer after completion of treatment.

In some embodiments, the cancer is selected from prostate cancer, brain cancer, breast cancer, colorectal cancer, pancreatic cancer, ovarian cancer, lung cancer, cervical cancer, liver cancer, head/neck/throat cancer, skin cancer, bladder cancer, or a hematologic cancer. In some embodiments, the cancer is a solid tumor cancer selected from an adenoma, an adenocarcinoma, a blastoma, a carcinoma, a desmoid tumour, a desmopolastic small round cell tumour, an endocrine tumour, a germ cell tumour, a lymphoma, a leukaemia, a sarcoma, a Wilms tumour, a lung tumour, a colon tumour, a lymph tumour, a breast tumour, or a melanoma. In some embodiments, the cancer is metastatic melanoma.

In some embodiments, the subject has an immune profile indicative of response to treatment with the first immune checkpoint polypeptide or the polynucleotide encoding the same, the second immune checkpoint polypeptide or the polynucleotide encoding the same, and the immune checkpoint inhibitor.

In some embodiments, the present disclosure provides a method for treating a cancer in a subject in need thereof comprising administering to the subject: an IDO immune checkpoint polypeptide or a polynucleotide encoding the same, wherein the IDO polypeptide comprises or consists of the sequence of ALLEIASCL (SEQ ID NO: 2) or the sequence of DTLLKALLEIASCLEKALQVF (SEQ ID NO: 3); a PD-L1 immune checkpoint polypeptide or a polynucleotide encoding the same, wherein the PD-L1 polypeptide comprises or consists of the sequence of FMTYWHLLNAFTVTVPKDL (SEQ ID NO: 32); and an immune checkpoint inhibitor, wherein the immune checkpoint inhibitor is an anti-PD1 antibody.

In some embodiments, the present disclosure provides a method of preventing disease progression in a subject suffering from a cancer comprising administering to the subject: an IDO immune checkpoint polypeptide or a polynucleotide encoding the same, wherein the IDO polypeptide comprises or consists of the sequence of ALLEIASCL (SEQ ID NO: 2) or the sequence of DTLLKALLEIASCLEKALQVF (SEQ ID NO: 3); a PD-L1 immune checkpoint polypeptide or a polynucleotide encoding the same, wherein the PD-L1 polypeptide comprises or consists of the sequence of FMTYWHLLNAFTVTVPKDL (SEQ ID NO: 32); and an immune checkpoint inhibitor, wherein the immune checkpoint inhibitor is an anti-PD1 antibody.

In some embodiments, the present disclosure provides a method of reducing tumor volume in a subject suffering from a cancer comprising administering to the subject: an IDO immune checkpoint polypeptide or a polynucleotide encoding the same, wherein the IDO polypeptide comprises or consists of the sequence of ALLEIASCL (SEQ ID NO: 2) or the sequence of DTLLKALLEIASCLEKALQVF (SEQ ID NO: 3); a PD-L1 immune checkpoint polypeptide or a polynucleotide encoding the same, wherein the PD-L1 polypeptide comprises or consists of the sequence of FMTYWHLLLNAFTVTVPKDL (SEQ ID NO: 32); and an immune checkpoint inhibitor, wherein the immune checkpoint inhibitor is an anti-PD1 antibody.

In some embodiments, the subject has an immune profile indicative of response to treatment with the IDO polypeptide or polynucleotide encoding the same, the PD-L1 polypeptide or polynucleotide encoding the same, and the anti-PD1 antibody.

In some embodiments, the present disclosure provides a kit comprising: a first immune checkpoint polypeptide or a polynucleotide encoding the same; a second immune checkpoint polypeptide or a polynucleotide encoding the same; and an immune checkpoint inhibitor.

In some embodiments, first and second immune checkpoint polypeptides or polynucleotides encoding the same are provided as a single composition, in a separate sealed container from immune checkpoint inhibitor.

In some embodiments, the present disclosure provides an immunotherapeutic composition for use in a method for the prevention or treatment of cancer in a subject, wherein the immunotherapeutic composition comprises a first immune checkpoint polypeptide or a polynucleotide encoding the same and a second immune checkpoint polypeptide or a polynucleotide encoding the same.

In some embodiments, the present disclosure provides use of an immunotherapeutic composition in the manufacture of a medicament for the prevention or treatment of cancer in a subject, the immunotherapeutic composition comprising a first immune checkpoint polypeptide or a polynucleotide encoding the same and a second immune checkpoint polypeptide or a polynucleotide encoding the same, which is formulated for administration before, concurrently with, and/or after an immune checkpoint inhibitor.

In some embodiments, the subject has an immune profile indicative of response to treatment with the first immune checkpoint polypeptide or the polynucleotide encoding the same, the second immune checkpoint polypeptide or the polynucleotide encoding the same, and the immune checkpoint inhibitor. In some embodiments, wherein the first immune polypeptide is an IDO1 polypeptide, the second immune polypeptide is a PD-L1 polypeptide and the immune checkpoint inhibitor is an antibody that binds to PD1.

In some embodiments, the immune profile comprises one or more of the following: a decrease in CD4+ T regulatory cells compared to a control subject group; a decrease in CD28+CD4+ T cells compared to a control subject group; an increase in LAG-3+CD4+ T cells compared to a control subject group; a decrease in monocytic-myeloid derived suppressor cells (mMDSCs) compared to a control subject group; an increase in CD56dimCD16+ Natural Killer (NK) cells compared to a control subject group; a decrease in CD56brightCD16− NK cells compared to a control subject group; and/or an increase in conventional dendritic cells type 2 (cDC2) compared to a control subject group.

In some embodiments, the cell populations are determined by FACS analysis of a peripheral blood sample obtained from the subject.

In some embodiments, the present disclosure provides a method for stratifying a cancer patient into one of at least two treatment groups, wherein said method comprises analysing one or more cell populations in a peripheral blood sample from the patient to determine an immune profile; and: i. stratifying the patient into a first treatment group if the immune profile determined is indicative of response to treatment with an IDO1 polypeptide, a PD-L1 polypeptide and an antibody that binds to PD1; or ii. stratifying the patient into a second treatment group if the immune profile determined in step indicates that the subject will not respond to said treatment.

In some embodiments, the first treatment group is to be treated, or is treated with, the IDO1 polypeptide, the PD-L1 polypeptide and the antibody that binds to PD1 and the second treatment group is to be treated with, or is treated with, one or more alternative therapies. In some embodiments, the immune profile is a baseline immune profile.

In some embodiments, the immune profile indicative of response to treatment comprises or more of: a) a decrease in CD4+ T regulatory cells compared to a control subject population; b) a decrease in CD28+CD4+ T cells compared to a control subject population; c) an increase in LAG-3+CD4+ T cells compared to a control subject population; d) a decrease in monocytic-myeloid derived suppressor cells (mMDSCs), compared to a control subject population; e) an increase in CD56^{dim}CD16+ Natural Killer (NK) cells compared to a control subject population; f) a decrease in CD56^{bright}CD16-Natural Killer (NK) cells compared to a control subject population; and g) an increase in conventional dendritic cells type 2 (cDC2) compared to a control subject population.

In some embodiments, the present disclosure provides a method of monitoring the response of a cancer patient to treatment with an IDO1 polypeptide, a PD-L1 polypeptide and an antibody that binds to PD1, wherein said method comprises analysing one or more cell populations in a peripheral blood sample from the patient to determine an immune profile, and: i. determining that the patient is responding to treatment if the patient has an immune profile indicative of response to treatment; or ii. determining that the patient is not responding to treatment if the patient does not have an immune profile indicative of response to treatment.

In some embodiments, the immune profile indicative of response to treatment comprises increased expression of CD28, HLA-DR, CD39, TIGIT and/or T4-3 on CD4+ T cells and/or increased expression of HLA-DR, CD39, LAG-3 and/or TIGIT on CD8+ T-cells.

In some embodiments, the cancer patient has a metastatic melanoma.

### BRIEF DESCRIPTION OF THE FIGURES

FIG. 1A-FIG. 1B provide schematics describing the methods provided herein. FIG. 1A illustrates the hypothesized mechanism of action for the combination immunotherapy regimen described herein (e.g., IDO/PD-L1 peptide vaccine and nivolumab (anti-PD1)). FIG. 1B provides a schematic of the combination immunotherapy regimen described herein.

FIG. 2A-FIG. 2F show various clinical responses. FIG. 2A provides percentages of overall response rate (ORR), complete response (CR), partial response (PR), and progressive disease (PD) according to RECIST 1.1 by investigator review in all patients (n=30). FIG. 2B illustrates treatment effect in MM1636 compared with a matched historical control group from the DAMMED database (n=74). FIG. 2C provides the best change in the sum of target lesion compared with baseline (n=30). FIG. 2D provides Kaplan-Meier curve of duration of response in the 24 patients with an objective response. FIG. 2E provides Kaplan-Meier curve of progression free survival in all 30 treated patients. FIG. 2F provides Kaplan-Meier curve of overall survival in all 30 treated patients.

FIG. 3A-FIG. 3C illustrate duration and kinetics of responses. FIG. 3A provides a Swimmer plot showing duration of response and time to response according to RECIST 1.1 of all treated patients (n=30). Triangle indicates first evidence of partial response while a square indicate first evidence of complete response. Closed circles indicate time of progression. Arrow indicates ongoing response. Patient MM18 died due to nivolumab induced side effects. FIG. 3B provides a Spider plot showing response kinetics in all treated patients (n=30). Red square indicates time of progression. FIG. 2C provides PET/CT images of patient MM42 pre- and post-treatment (after 12 series of treatment) showing FDG-metabolism in target lesions.

FIG. 4 provides bar plots indicating HLA-genotype and clinical response. Blue bars represent responders (CR+PR) and orange bars represent non-responders (PD).

FIG. 5A-FIG. 5D illustrates vaccine specific responses in blood. FIG. 5A illustrates IDO and PD-L1 specific T-cell responses in PBMCs at baseline and on vaccination as measured by IFN-γ Elispot assay. FIG. 5B illustrates IDO and PD-L1 specific T-cells response in PBMC in all treated patients measured by IFN-γ Elispot assay at baseline and on vaccination. FIG. 5C provides a representative example of Elispot-wells with response in patient MM23 in serial PBMCs before and on treatment. FIG. 5D illustrates the cytolytic potential of in vitro stimulated and sorted IDO specific CD4 and CD8 T-cells from blood in patient MM14 on vaccination shown by the expression of CD107a, IFN-γ and TNF-α

FIG. 6 illustrates vaccine related adverse events.

FIG. 7A-FIG. 7D illustrates T-cell changes in blood after treatment. FIG. 7A illustrates T-cell fraction in peripheral blood of five patients at baseline, series 3, 6 and 12 by TCR sequencing. T-cell fraction was calculated by taking the total number of T-cell templates and dividing by the total number of nucleated cells. FIG. 7B illustrates TCR clonality in peripheral blood of five patients at baseline, series 3, 6 and 12 by TCR sequencing. FIG. 7C illustrates TCR repertoire richness in peripheral blood of five patients at baseline, series 3, 6 and 12 by TCR sequencing. FIG. 7D provides a bar-chart representing peripherally expanded clones in five patients at series 3, series 6 and series 12. Light gray bar represents peripherally expanded clones that are present in baseline biopsy while dark grey bar represents peripherally expanded clones that are present in the post-treatment biopsy (after 6 series).

FIG. 8A-8C illustrate Vaccine specific responses in blood. FIG. 8A provides a heatmap of specific (background has been subtracted) IDO and PD-L1 responses in PBMCs at baseline, series 3, 6, 12, 18 and 24 measured by IFN-γ Elispot assay and shows fluctuations in the blood during treatment. FIG. 8B provides a heatmap of specific (background has been subtracted) IDO and PD-L1 responses in PBMCs at baseline and 3 and 6 months after last vaccine measured by an IFN-γ Elispot assay. FIG. 8C illustrates vaccine associated clones were tracked by summing the frequency of each rearrangement enriched in either IDO or PD-L1 T-cells

FIG. 9A-FIG. 9G illustrate changes in the tumour microenvironment after treatment, including number of CD3 and CD8 T-cells, TCR fraction, TCR clonality, TCR repertoire, biopsy expanded TCR clones and enrichment of IDO and PD-L1 specific T cells at the tumor site. FIG. 9A provides the number of CD3+ and CD8+ T-cells/mm² at the tumour site detected by IHC on paired biopsies from 4 patients. FIG. 9B illustrates T-cell fraction at the tumour site pre- and post-treatment (after series 6) by TCR sequencing. T-cell fraction was calculated by taking the total number of T-cell templates and dividing by the total number of nucleated cells. FIG. 9C illustrates representative example of IHC of CD3+ and CD8+ T-cells at the tumour site before and after treatment in patient MM01.

FIG. 9D illustrates tracking of vaccine associated clones in pre- and post-treatment tumour biopsies. Cumulative frequencies of IDO and PD-L1 vaccine specific TCR rearrangements are represented. FIG. 9D and FIG. 9F provide TCR clonality and TCR repertoire richness in 5 patients at the tumour site before and after treatment. FIG. 9G provides a bar-chart representing baseline expanded biopsy clones on five patients and the detection of biopsy expanded clones also found in the blood at baseline, series 3, 6 and 12 by TCR sequencing.

FIG. 10A-10D illustrate pro-inflammatory profiles of sorted CD4 and CD8 T cells from blood. FIG. 10A provides percentage of in vitro stimulated and sorted CD4+PD-L1 specific T cells that are positive for CD107a and percentages that secrete IFN-γ and/or TNFα. FIG. 10B provides percentage of in vitro stimulated and sorted CD8+PD-L1 specific T cells that are positive for CD107a and percentages that secrete IFN-γ and/or TNFα. FIG. 10C provides percentage of in vitro stimulated and sorted CD4+ IDO-specific T cells that are positive for CD107a and percentages that secrete IFN-γ and/or TNFα. FIG. 10D provides percentage of in vitro stimulated and sorted CD8+ IDO-specific T cells that are positive for CD107a and percentages that secrete IFN-γ and/or TNFα.

FIG. 11A-FIG. 11B illustrate treatment induced upregulation of PD-L1, IDO, MHC I and MHC II and distance between CD8 T cells and PD-L1 expressing cells. FIG. 11A provides IHC on 4 paired biopsies stained for CD3+ and CD8+ T-cells, PD-L1, MHCI and MHCII on tumor cells/mm² and IDO H-score (expression of IDO on both immune and tumor cells.) FIG. 11B provides distance in m between CD8+ T-cells and PD-L1+ stained cells on five baseline biopsies detected by IHC.

FIG. 12A-FIG. 12D illustrates vaccine-specific responses in skin. FIG. 12A illustrates IDO and PD-L1 specific T-cell responses in SKILs after 6 series of treatment measured by IFN-γ Elispot assay. FIGS. 12B, 12C, and 12D show percentage of cytokine secreting/CD107a CD4+ and CD8+ IDO and PD-L1 specific T-cells in response to in vitro peptide stimulation by flow cytometry.

FIG. 13A-FIG. 13B illustrates infiltrating PD-L1 specific T cell clones also found in biopsy in patient MM01. FIG. 13A illustrates TCR sequencing performed on a PD-L1 specific T cell culture generated from DTH area on the lower back injected with PD-L1 peptide on patient MM01. Bars show the frequency of top 25 clones in the culture with which indicates a high Simpson clonality of 0.43. FIG. 13B demonstrates tracking the frequency of the top five skin infiltrating PD-L1 specific clones in tumour before and after treatment.

FIG. 14A-FIG. 14C illustrates profiling of genes relevant to T cell activation, cytokines and exhaustion markers on pre- and post-treatment biopsies in two patients. FIG. 14A provides RNA expression profiling of genes related to T cell activation was performed using NanoString nCounter. FIG. 14B provides RNA expression profiling of genes related to cytokine activity was performed using NanoString nCounter. FIG. 14C provides RNA expression profiling of genes related to checkpoint inhibitors was performed using NanoString nCounter.

FIG. 15 provides the gating strategy used in cytokine production profile of IDO and PD-L1 specific T cells by intracellular staining.

FIG. 16 provides baseline correlative signatures at tumor site indicative of clinical response. FIG. 16A illustrates the immune profiles for eight patient biopsies that were stained for CD3 and CD8 T-cells, PD-L1, MHC I and MHC II on tumour cells and IDO H-score (expression of IDO on both immune and tumour cells). FIG. 16B illustrates IHC with staining of the exhaustion markers PD-1, TIM-3 and LAG-3 on CD8 T-cells in multiple combinations (positive for 1, positive for 2 or positive for 3).

FIG. 17 illustrates baseline RNA gene expression profiling at tumor site of 776 genes relating related to innate and adaptive immunity.

FIG. 18 illustrates multicolour flow cytometry analysis performed to determine a baseline peripheral blood immune cell profile. FIG. 18A-FIG. 18D illustrate the difference between responders and non-responders in terms of: (A) percentage Tregs as a percentage of CD4+ T cells; (B) percentage cCD2 as a percentage of PBMCs; (C) percentage LAG3 as a percentage of CD4+ T cells; and (D) percentage CD28 as a percentage of CD4+ T cells.

FIG. 19 illustrates multicolour flow cytometry analysis performed to assess response of patients during treatment. Responders are shown in left-hand three columns of each plot (light grey shading). Non-responders are shown on right-hand three columns of each plot (dark grey shading). FIG. 19A shows percentage Tregs as a percentage of CD4+ T cells for thirty patients (responders and non-responders) at baseline, cycle 3 and cycle 6. FIG. 19B shows mMDSCs as a percentage of PBMCs for thirty patients (responders and non-responders) at baseline, cycle 3 and cycle 6. Wilcoxon matched paired rank t-test was used and * denotes p<0.05.

FIG. 20 illustrates Kynurenine/Tryptophan (Kyn/Trp) ratio at baseline and after treatment. FIG. 20A provides Kyn/Trp ratios shown at baseline for complete responders (CR), partial responders (PR) and patients with progressive disease (PD). FIG. 20B provides fold changes in kyn/trp ratio (log 1) shown from baseline to cycle 3 in complete responders (CR), partial responders (PR) and patients with progressive disease (PD).

FIG. 21 summarises the treatment schedule with biopsy and blood samples time points. Responders are shown in left-hand three columns of each plot (light grey shading). Non-responders are shown on right-hand three columns of each plot (dark grey shading). Patients were treated with the IDO/PD-L1 peptide vaccine every second week for the first 6 injections and thereafter every fourth week. Nivolumab (2 mg/kg) was administered every second week up to two years. Blood samples for research use was drawn at baseline, cycle 3, cycle 6, cycle 12 and thereafter every third month. Needle biopsies for research use was taken at baseline and at cycle 6. Circle indicates samples that have been used for analysis in this study.

FIG. 22 illustrates multicolour flow cytometry analysis performed to assess baseline peripheral blood immune cell subset differences between responders and non-responders. FIG. 22A-FIG. 22C show the differences between responders and non-responders in terms of: (A) mMDSCs as a percentage of PBMCs; (B) CD56^{dim} CD16+ cells as a percentage of PMBCs; and (C) CD56^{bright} CD16+ cells as a percentage of PMBCs.

FIG. 23 illustrates multicolour flow cytometry performed to assess treatment-induced changes in immune status of CD4+ T cells. FIG. 23A-FIG. 23E show the differences between responders and non-responders at baseline, cycle 3 and cycle 6 in terms of percentages of: (A) CD28+CD4+ T cells; (B) HLA-DR+ CD4+ T cells; (C) CD39+ CD4+ T cells; (D) TIGIT+ CD4+ T cells; and (E) TIM-3+CD4+ T cells.

FIG. 24 illustrates multicolour flow cytometry performed to assess treatment-induced changes in immune status of CD8+ T cells. FIG. 23A-FIG. 23D show the differences between responders and non-responders at baseline, cycle 3 and cycle 6 in terms of percentages of: (A) HLA-DR+CD8+ T cells; (B) CD39+CD8+ T cells; (C) LAG-3+CD8+ T cells; and (D) TIGIT+CD8+ T cells.

FIG. 25 provides a gating strategy for T cell differentiation.

FIG. 26 provides a gating strategy for inhibitory and activation molecules on T cells.

FIG. 27 provides a universal gating strategy.

FIG. 28 provides a summary of the antibodies used for flow cytometry.

FIG. 29 illustrates progression free survival and overall survival in matched historical control group. FIG. 29A provides Kaplan-Meier curve of progression free survival in the matched historical control group (n=74). Patients were matched on BRAF-status, PD-L1− status, age, gender, M-stage and LDH level). FIG. 29B provides Kaplan-Meier curve of overall survival in the matched historical control group (n=74).

FIG. 30 illustrates CD4 and CD8 vaccine specific T-cell responses in blood. Top: heatmaps of IDO specific CD4 (FIG. 30A) and CD8 (FIG. 30B) T-cell responses in PBMCs at baseline and on/post treatment. Bottom: heatmaps of IDO specific CD4 (FIG. 30A) and CD8 (FIG. 30B) T cell responses. Responses quantified by flow cytometry by an increased expression of CD107α, CD137 and TNFα after 8 h peptide stimulation. Values represent specific responses after background values have been subtracted; n=21.

FIG. 31 illustrates T-cell changes in blood after treatment. T-cell fraction, TCR clonality and repertoire richness in blood and peripherally expanded TCR clones in both responding and non-responding patients. FIG. 31A indicates T-cell fraction in peripheral blood of five patients at baseline, series 3, 6 and 12 by TCR sequencing. T-cell fraction was calculated by taking the total number of T-cell templates and dividing by the total number of nucleated cells. FIG. 31B indicates TCR clonality in peripheral blood of five patients at baseline, series 3, 6 and 12 by TCR sequencing. Simpson clonality measures how evenly TCR sequences are distributed amongst a set of T-cells where 0 indicate even distribution of frequencies and 1 indicate an asymmetric distribution where a few clones dominate. FIG. 31C indicates TCR repertoire richness in peripheral blood of five patients at baseline, series 3, 6 and 12 by TCR sequencing. TCR repertoire richness report the mean number of unique rearrangements. FIG. 31D shows a bar-chart chart representing dynamics of the expanded T cell clones. Wide rear bars represent peripherally expanded clones in five patients at series 3, series 6 and series 12 as compared to the baseline PBMC samples. MM01 was CR. MM08 and MM13 were PR. MM02 and MM09 were PD. Superimposed light grey bar represents peripherally expanded clones that are present in baseline biopsy samples while the superimposed dark grey bar represents peripherally expanded clones that are present in the post-treatment biopsy (after 6 series). FIG. 31E indicates the frequency of the dominant TCR R chain in clonal PD-L1 and IDO specific cultures as determined by CDR3 sequencing.

FIG. 32 illustrates PD-L1 and IDO specific T cells from vaccinated patients react against PD-L1 and IDO expressing target cells. FIG. 32A: Left: PD-L1 specific T cell culture (MM1636.05) reactivity against PD-L1 peptide or autologous tumor cells in IFNγ ELISPOT. Tumour cells were either non-treated or pre-treated with 200 U/ml IFNγ for 48 h prior to assay. Right: (Top) PD-L1 surface expression on melanoma cell with (green; right-most peak) or without pre-treatment (yellow; central and largest peak) with IFNγ compared to isotype control (grey; left-most peak) as assed by flow cytometry; (Bottom) HLA II surface expression on melanoma cell with (green; right-most peak) or without pre-treatment (yellow; left aligned and largest peak) with IFNγ compared to isotype control (grey; left aligned and smallest peak) as assed by flow cytometry. FIG. 32B: Left: PD-L1 specific T cell (MM1636.05) reactivity in IFNγ ELISPOT assay against autologous tumour cells pre-treated with IFNγ (500 U/ml) and transfected with Mock or PD-L1 siRNA 24 h after transfection. Right: PD-L1 surface expression on melanoma tumour cells (MM1636.05) assessed by flow cytometry 24 h after transfection with Mock (blue; right-most and smallest peak) or PD-L1 (red; central peak of intermediate size) siRNA, compared to the isotype control (grey; left most and largest peak). FIG. 32C illustrates reactivity of CD4+PD-L1 specific T cell clone (MM1636.14) against PD-L1 peptide or autologous CD14+ cells; E:T ratio 10:1. CD14+ cells were isolated using magnetic bead sorting and used as targets in an ELISPOT assay directly or after 2 day pre-treatment with tumour conditioned media (TCM) derived from autologous tumor cell line. FIG. 32D illustrates RT-qPCR analysis of PD-L1 expression in sorted CD14+ cells before and after treatment with autologous TCM for 48 h. FIG. 32E illustrates reactivity of IDO specific CD4+ T cell clone (MM1636.23) against IDO peptide combined with HLA-DR (L243), HLA-DQ (SPV-L3) or HLA-DP (B7/21) blocking antibodies in intracellular staining assay for IFNγ and TNFα production. T cells incubated with 2 μg/mL of the individual blocking antibodies for 30 min prior to addition of IDO peptide. FIG. 32F illustrates IDO specific CD4+ T cell clone (MM1636.23) reactivity against HLA-DR matched IDO expressing cell line MonoMac1 transfected with Mock or IDO siRNA in ICS assay. siRNA transfection performed 48 h prior to the experiment. g) RT-qPCR analysis of IDO1 expression in MonoMac1 48 h after siRNA transfection. FIG. 32H illustrates reactivity of CD4+ IDO specific T cell clone (MM1636.14) against IDO peptide or autologous CD14+ cells; E:T ratio 20:1. CD14+ cells were isolated using magnetic bead sorting and used as targets in an ELISPOT assay directly or after pre-treatment with TCM derived from autologous tumour cell line. FIG. 32I illustrates RT-qPCR analysis of IDO1 expression in sorted CD14+ cells before and after treatment with autologous TCM for 48 h. In FIGS. 32D, 33G and 33I, RT-qPCR data bars represent mean of 3 or 6 technical replicates ±SD; P values determined by two-tailed parametric t test. In FIG. 32A-C and FIG. 32H, ELISPOT counts represent the mean value of 3 technical replicates ±SEM; response P values determined using DFR method. Bars labelled with ‘TNTC’ indicates that the number of IFNγ producing cells was too numerous to count.

FIG. 33 illustrates ex vivo vaccine specific responses in blood. FIGS. 33A and 33B provide heatmaps of detected specific (background has been subtracted) IDO (A) and PD-L1 (B) responses in PBMCs at baseline and on/after treatment as measured by IFNγ ELISPOT (n=25). FIG. 33C provides example well images of ex vivo ELISPOT wells for three different patients. 6-9×10⁵ cells per well were used.

FIG. 34 provides the gating strategy used for data presented in FIG. 30 in relation to the assessment of IDO and PD-L1 specific T cells by intracellular staining of PBMCs ex vivo.

### BRIEF DESCRIPTION OF THE SEQUENCE LISTING

SEQ ID NO: 1 is the amino acid sequence of human Indoleamine 2,3-dioxygenase (IDO1). SEQ ID NO: 2 is the amino acid sequence of a fragment of IDO1, referred to herein as 10101 or IDO5. SEQ ID NO: 3 is the amino acid sequence of a fragment of IDO1, referred to herein as 10102. SEQ ID NOs 4 to 13, are the amino acid sequences of other fragments of IDO1 disclosed herein. SEQ ID NO: 14 is the amino acid sequence of human PD-L1. SEQ ID NOs: 15 to 31 and 32 are the amino acid sequences of fragments of PD-L1 disclosed herein. SEQ ID NO: 32 is the amino acid sequence of a fragment of PD-L1 which may be referred to herein as 10103. SEQ ID NOs: 33 and 34 are the nucleotide sequences for a PD-L1 siRNA duplex. SEQ ID NOs: 35 to 40 are the nucleotide sequences for the IDO siRNA duplexes designated siRNA1, 2 and 3.

### DETAILED DESCRIPTION

### Overview

The antitumor activity of PD1 monotherapy may be compromised by a limited pool of pre-existing naive and primed tumour-specific T-cells. Immune modulatory vaccines targeting tumoral immune escape mechanisms offer a treatment strategy that is applicable to general patient populations, as these escape mechanisms are found in numerous cancer types and across diverse patient populations. This is in contrast to patient-specific-neoantigen cancer vaccines, which are specifically tailored to a particular tumour and are not broadly applicable (Ott, P. A. et al. Cell 740 183, 347-362.e24 (2020); Andersen, M. H. Semin. Immunopathol. 41, 1-3 (2019)).

Circulating cytotoxic T-cells against two immune checkpoint molecules, indoleamine 2,3-dioxygenase (IDO) and programmed death-ligand 1 (PD-L1), have been detected in the blood of cancer patients and, to a lesser extent, healthy donors (Munir, S. et al., Oncoimmunology 2, e23991 (2013); Ahmad, S. et al., Cancer Immunol. Immunother. 65, 797-804 (2016); Andersen, M. Cancer Immunol. Immunother. 61, 1289-1297 (2012); Sorensen, Cancer Res. 71, 2038-2044 (2011); Ahmad, S., et al., Leukemia 28, 236-8 (2014); Andersen, M. H. Oncoimmunology 1, 1211-1212 (2012)). These T-cells directly recognize tumour cells as well as immunosuppressive cells in the tumour microenvironment and can therefore be exploited to restrict the range of immunosuppressive signals and reverse the immunosuppressive nature of the tumour microenvironment. Thus, the IDO/PD-L1 immune-modulating vaccine strategy described herein may lead to a translatable strategy for improving the efficacy of αPD1 therapy through activation of these specific T-cells.

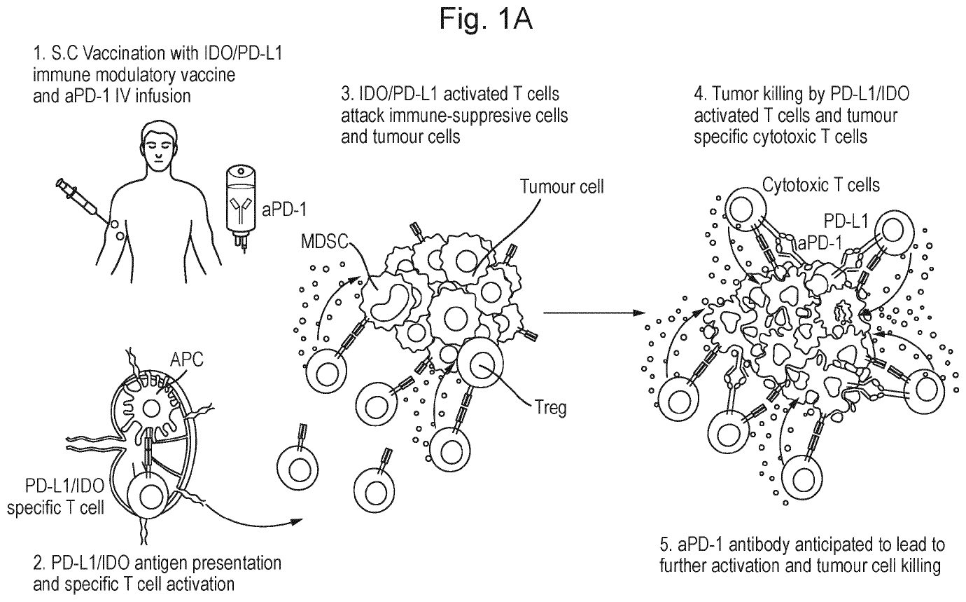

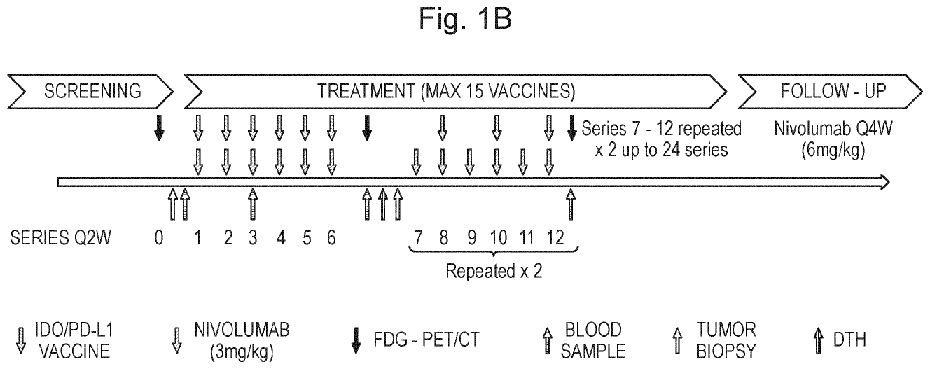

While not intending to be limiting on the present disclosure, the hypothesized mechanism of action is depicted in FIG. 1A. Briefly, (1) the IDO/PD-L1 vaccine and αPD1 antibody are administered to the patient. (2) The peptide vaccine is phagocytosed by antigen presenting cells and presented to IDO and PD-L1 specific T cells, which are then activated. (3) The activated T cells migrate into the tumour environment where they attack both immunosuppressive cells and tumor cells leading to cytokine production and a pro-inflammatory Th1-driven tumor microenvironment, thereby reversing the immunosuppressive nature of tumour microenvironment into an immune permissive environment. (4/5) This increase in inflammatory signalling also leads to a parallel upregulation of PD-1 expression in cancer and immune cells, thereby leading to enhanced tumor killing by both IDO/PD-L1 specific T cells and tumor specific cytotoxic T cells due to PD-1 blockade.

### Definitions

It is to be understood that different applications of the disclosed products and methods may be tailored to the specific needs in the art. It is also to be understood that the terminology used herein is for the purpose of describing particular embodiments of the invention only and is not intended to be limiting.

In addition, as used in this specification and the appended claims, the singular forms “a”, “an”, and “the” include plural referents unless the content clearly dictates otherwise. Thus, for example, reference to “an inhibitor” includes two or more such inhibitors, or reference to “an oligonucleotide” includes two or more such oligonucleotide and the like.

As used herein, the term “effective amount” refers to the minimum amount of an agent or composition required to result in a particular physiological effect. The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may also be expressed as the half-maximal effective concentration (EC₅₀), which refers to the concentration of an agent that results in a magnitude of a particular physiological response that is half-way between a reference level and a maximum response level.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

The terms “prevent,” “prophylaxis,” and “prophylactically” refer to the administration of a compound prior to the onset of disease (e.g., prior to the onset of certain symptoms of a disease). Preventing disease may include reducing the likelihood that the disease will occur, delaying onset of the disease, ameliorating long term symptoms, or delaying eventual progression of the disease.

A “subject” as used herein includes any mammal, preferably a human.

As used herein, the terms “treatment,” “treating,” or “ameliorating” refers to either a therapeutic treatment or prophylactic/preventative treatment. A treatment is therapeutic if at least one symptom of disease in an individual receiving treatment improves or a treatment can delay worsening of a progressive disease in an individual or prevent onset of additional associated diseases.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogs, and peptidomimetics.

### Immune Checkpoint Polypeptides

The present disclosure provides methods for treating a cancer in a subject in need thereof comprising administering to the subject: (a) a first immune checkpoint peptide or a polynucleotide encoding the same; (b) a second immune checkpoint peptide or a polynucleotide encoding the same; and (c) an immune checkpoint inhibitor.

Effector T cell activation is normally triggered by the T cell receptor recognising antigenic peptide presented by the MHC complex. The type and level of activation achieved is then determined by the balance between signals which stimulate and signals which inhibit the effector T cell response. The term “immune checkpoint molecule” is used herein to refer to component of the human immune system, typically a molecule comprising a mechanism of action that alters the balance in favour of inhibition of the effector T cell response. For example, a molecule which, upon interacting with its ligand, negatively regulates the activation of an effector T cell. Such regulation might be direct, such as by the interaction between a ligand and a cell surface receptor which transmits an inhibitory signal into an effector T cell. Such regulation might be indirect, such as by the blocking or inhibition of an interaction between a ligand and a cell surface receptor which would otherwise transmit an activating signal into the effector T cell, or an interaction which promotes the upregulation of an inhibitory molecule or cell, or the depletion by an enzyme of a metabolite required by the effector T cell, or any combination thereof. The term “immune checkpoint polypeptide” refers to a polypeptide sequence of an immune checkpoint molecule that is capable of inducing an immune response against the immune checkpoint molecule when administered to a subject. The immune checkpoint polypeptides described herein may comprise the full-length amino acid sequence of an immune checkpoint molecule. In particular embodiments, the immune checkpoint polypeptides comprise an immunogenic fragment of the immune checkpoint molecule. An “immunogenic fragment” is used herein to mean a polypeptide fragment that is shorter than the full amino acid sequence of the immune checkpoint molecule, but which is capable of eliciting an immune response to the immune checkpoint molecule.

The ability of a fragment to elicit an immune response (i.e., the “immunogenicity” of a polypeptide or fragment thereof) to immune checkpoint molecule may be assessed by any suitable method. Typically, the fragment will be capable of inducing proliferation and/or cytokine release in vitro in T cells specific for the immune checkpoint molecule, wherein said cells may be present in a sample of lymphocytes taken from a cancer patient. Proliferation and/or cytokine release may be assessed by any suitable method, including ELISA and ELISPOT. Exemplary methods are described in the Examples. In some embodiments, the fragment induces proliferation of component-specific T cells and/or induces the release of IFNγ and/or TNFα from such cells.

In order to induce proliferation and/or cytokine release in T cells specific for the immune checkpoint molecule, the fragment must be capable of binding to an MHC molecule such that it is presented to a T cell. In other words, the immune checkpoint polypeptides and fragments thereof comprise or consist of at least one MHC binding epitope of the said component. Said epitope may be an MHC Class I binding epitope or an MHC Class II binding epitope. In some embodiments, the immune checkpoint polypeptides and fragments thereof comprise more than one MHC binding epitope, each of which said epitopes binds to an MHC molecule expressed from a different HLA-allele, thereby increasing the breadth of coverage of subjects taken from an outbred human population.

MHC binding may be evaluated by any suitable method including the use of in silico methods. Preferred methods include competitive inhibition assays wherein binding is measured relative to a reference peptide. The reference peptide is typically a peptide which is known to be a strong binder for a given MHC molecule. In such an assay, a peptide is a weak binder for a given HLA molecule if it has an IC50 more than 100-fold lower than the reference peptide for the given HLA molecule. A peptide is a moderate binder is it has an IC50 more than 20-fold lower but less than a 100-fold lower than the reference peptide for the given HLA molecule. A peptide is a strong binder if it has an IC50 less than 20-fold lower than the reference peptide for the given HLA molecule.

A fragment comprising an MHC Class I epitope preferably binds to a MHC Class I HLA species selected from the group consisting of HLA-A1, HLA-A2, HLA-A3, HLA-A11 and HLA-A24, more preferably HLA-A3 or HLA-A2. Alternatively, the fragment may bind to a MHC Class I HLA-B species selected from the group consisting of HLA-B7, HLA-B35, HLA-B44, HLA-B8, HLA-B15, HLA-B27 and HLA-B51.

A fragment comprising an MHC Class II epitope preferably binds to a MHC Class II HLA species selected from the group consisting of HLA-DPA-1, HLA-DPB-1, HLA-DQA1, HLA-DQB1, HLA-DRA, HLA-DRB and all alleles in these groups and HLA-DM, HLA-DO.

Examples of immune checkpoint molecules include Indoleamine 2,3-dioxygenase (IDO1), PD-1 and PD-L1 or PD-L2, CTLA4, CD86, CD80, B7-H3 and/or B7-H4 and their respective ligands, HVEM and BTLA, GAL9 and TIM3, LAG3, and KIR.

In some embodiments, the immune checkpoint polypeptide is an IDO1 polypeptide. IDO1 is upregulated in cells of many tumours and is responsible for catalyzing the conversion of L-tryptophan to N-formylkynurenine and is thus the first and rate limiting enzyme of tryptophan catabolism through the Kynurenine pathway. This checkpoint is the metabolic pathway in cells of the immune system requiring the essential amino acid tryptophan. A lack of tryptophan results in the general suppression of effector T cell functions and promotes the conversion of naive T cells into regulatory (i.e. immunosuppressive) T cells (Tregs).

In some embodiments, the IDO1 immune checkpoint polypeptide elicits an immune response against IDO1. The IDO1 immune checkpoint polypeptide may thus alternatively be described as a vaccine against IDO1. Vaccines against IDO1 which may be used in the immunotherapeutic compositions provided herein are described in WO2009/143843; Andersen and Svane (2015), Oncoimmunology Vol 4, Issue 1, e983770; and Iversen et al (2014), Clin Cancer Res, Vol 20, Issue 1, p 221-32.

The IDO1 immune checkpoint polypeptide may comprise IDO1 (SEQ ID NO: 1) or an immunogenic fragment thereof (e.g., any one of SEQ ID NOs: 2-13). The IDO1 immunogenic fragment may comprise at least 8, preferably at least 9 consecutive amino acids of IDO1 (SEQ ID NO: 1). The said fragment may comprise up to 40 consecutive amino acids of IDO1 (SEQ ID NO: 1), up to 30 consecutive amino acids of IDO1 (SEQ ID NO: 1), or up to 25 consecutive amino acids of IDO1 (SEQ ID NO: 1). Thus, the fragment may comprise or consist of 8 to 40, 8 to 30, 8 to 25, 9 to 40, 9 to 30, or 9 to 25 consecutive amino acids of IDO1 (SEQ ID NO: 1). In some embodiments, the IDO1 fragment comprises or consists of 9 to 25 consecutive amino acids of IDO1 (SEQ ID NO: 1). Exemplary amino acid sequences of IDO1 fragments suitable for use according to the present disclosure are provided below in Table A.

TABLE A
Exemplary IDO1 polypeptide sequences

| | | AA positions | SEQ |
|---|---|---|---|
| Description | AA Sequence | in SEQ ID: 1 | ID: |
| IDO1 Full length | MAHAMENSWTISKEYHIDEEVGFALPNPQENLPDFYNDW | All | 1 |
| sequence | MFIAKHLPDLIESGQLRERVEKLNMLSIDHLTDHKSQRL | | |
| | ARLVLGCITMAYVWGKGHGDVRKVLPRNIAVPYCQLSKK | | |
| | LELPPILVYADCVLANWKKKDPNKPLTYENMDVLFSFRD | | |
| | GDCSKGFFLVSLLVEIAAASAIKVIPTVFKAMQMQERDT | | |
| | LLKALLEIASCLEKALQVFHQIHDHVNPKAFFSVLRIYL | | |
| | SGWKGNPQLSDGLVYEGFWEDPKEFAGGSAGQSSVFQCF | | |
| | DVLLGIQQTAGGGHAAQFLQDMRRYMPPAHRNFLCSLES | | |
| | NPSVREFVLSKGDAGLREAYDACVKALVSLRSYHLQIVT | | |
| | KYILIPASQQPKENKTSEDPSKLEAKGTGGTDLMNFLKT | | |
| | VRSTTEKSLLKEG | | |
| IDO1 Fragment | ALLEIASCL | [199-207] | 2 |
| (IO101) | | | |
| IDO1 Fragment | DTLLKALLEIASCLEKALQVE | [194-214] | 3 |
| (IO102) | | | |
| IDO1 Fragment | QLRERVEKL | [54-62] | 4 |
| (IOx1) | | | |
| IDO1 Fragment | FLVSLLVEI | [164-172] | 5 |
| (IOx2) | | | |
| IDO1 Fragment | TLLKALLEI | [195-203] | 6 |
| (IOx3) | | | |
| IDO1 Fragment | FIAKHLPDL | [41-49] | 7 |
| (IOx4) | | | |
| IDO1 Fragment | VLSKGDAGL | [320-328] | 8 |
| (IOx6) | | | |
| IDO1 Fragment | DLMNFLKTV | [383-391] | 9 |
| (IOx7) | | | |
| IDO1 Fragment | VLLGIQQTA | [275-283] | 10 |
| (IOx8) | | | |
| IDO1 Fragment | KVLPRNIAV | [101-109] | 11 |
| (IOx9) | | | |
| IDO1 Fragment | KLNMLSIDHL | [61-70] | 12 |
| (IOx10) | | | |
| IDO1 Fragment | SLRSYHLQIV | [341-350] | 13 |
| (IOx11) | | | |

In some embodiments, the IDO1 immune checkpoint polypeptide fragment comprises or consists of the amino acid sequence of SEQ TD NO: 2 or SEQ TD NO: 3. In some embodiments, the IDO1 immune checkpoint polypeptide fragment comprises or consists of the amino acid sequence SEQ TD NO: 3. An IDO1 immune checkpoint polypeptide fragment comprising or consisting of SEQ TD NO: 2 binds well to TILA-A2, which is a particularly common species of HLA. An IDO1 immune checkpoint polypeptide consisting of SEQ ID NO: 3 binds well to at least one of the specific class I and class II HLA species mentioned above. A fragment which comprises or consists of the amino acid sequence of SEQ TD NO: 2 or SEQ TD NO: 3 is advantageous in that it will be effective in a high proportion of the outbred human population.

In some embodiments, the immune checkpoint polypeptide is a PD-L1 polypeptide. PD-1 is expressed on effector T cells and engagement either PD-L1 or PD-L2 results in a signal which downregulates activation. The PD-L1 or PD-L2 ligands are expressed by some tumours. PD-L1 in particular is expressed by many solid tumours, including melanoma. These tumours may therefore down regulate immune mediated anti-tumour effects through activation of the inhibitory PD-1 receptors on T cells. By blocking the interaction between PD1 and one or both of its ligands, a checkpoint of the immune response may be removed, leading to augmented anti-tumour T cell responses.

In some embodiments, the PD-L1 immune checkpoint polypeptide elicits an immune response against PD-L1. The PD-L1 immune checkpoint polypeptide may thus alternatively be described as a vaccine against PD-L1. Vaccines against PD-L1 which may be used in the immunotherapeutic compositions provided herein are described in WO2013/056716.

The PD-L1 immune checkpoint polypeptide may comprise PD-L1 (SEQ ID NO: 14) or an immunogenic fragment thereof (e.g., any one of SEQ ID NOs: 15-32). The PD-L1 immunogenic fragment may comprise at least 8 or at least 9 consecutive amino acids of PD-L1 (SEQ ID NO: 14). The PD-L1 immunogenic fragment may comprise up to 40 consecutive amino acids of PD-L1 (SEQ ID NO: 14), up to 30 consecutive amino acids of PD-L1 (SEQ ID NO: 14), or. In some embodiments, the PD-L1 immunogenic fragment comprises up to 25 consecutive amino acids of PD-L1 (SEQ ID NO: 14). In some embodiments, the PD-L1 immunogenic fragment comprises or consists of 8 to 40, 8 to 30, 8 to 25, 9 to 40, 9 to 30, or 9 to 25 consecutive amino acids of PD-L1 (SEQ ID NO: 14). In some embodiments, the PD-L1 immunogenic fragment comprises or consists of 9 to 25 consecutive amino acids of PD-L1 (SEQ ID NO: 14).

Exemplary amino acid sequences of PD-L1 fragments suitable for use according to the present disclosure are provided below in Table B.

TABLE B
Exemplary PD-L1 polypeptide sequences

| | | AA positions in | |
|---|---|---|---|
| | | PDL1 | SEQ |
| Description | Sequence | (SEQ ID NO: 14) | ID: |
| PD-L1 Full | MRIFAVFIFMTYWHLLNAFTVTVPKDLYVVEYGSN | All | 14 |
| length | MTIECKFPVEKQLDLAALIVYWEMEDKNIIQFVHG | | |
| | EEDLKVQHSSYRQRARLLKDQLSLGNAALQITDVK | | |
| | LQDAGVYRCMISYGGADYKRITVKVNAPYNKINQR | | |
| | ILVVDPVTSEHELTCQAEGYPKAEVIWTSSDHQVL | | |
| | SGKTTTTNSKREEKLFNVTSTLRINTTTNEIFYCT | | |
| | FRRLDPEENHTAELVIPELPLAHPPNERTHLVILG | | |
| | AILLCLGVALTFIFRLRKGRMMDVKKCGIQDTNSK | | |
| | KQSDTHLEET | | |
| PD-L1 Fragment | LLNAFTVTV | 15-23 | 15 |
| (POL101) | | | |
| PD-L1 Fragment | ILLCLGVAL | 247-255 | 16 |
| (POL102) | | | |
| PD-L1 Fragment | ILGAILLCL | 243-251 | 17 |
| (POL103) | | | |
| PD-L1 Fragment | ALQITDVKL | 98-106 | 18 |
| (POL104) | | | |
| PD-L1 Fragment | KLFNVTSTL | 189-197 | 19 |
| (POL105) | | | |
| PD-L1 Fragment | RLLKDQLSL | 86-94 | 20 |
| (POL106) | | | |
| PD-L1 Fragment | QLSLGNAAL | 91-99 | 21 |
| (POL107) | | | |
| PD-L1 Fragment | KINQRILVV | 136-144 | 22 |
| (POL108) | | | |
| PD-L1 Fragment | HLVILGAIL | 240-248 | 23 |
| (POL109) | | | |
| PD-L1 Fragment | RINTTTNEI | 198-206 | 24 |
| (POL110) | | | |
| PD-L1 Fragment | CLGVALTFI | 250-258 | 25 |
| (POL111) | | | |
| PD-L1 Fragment | QLDLAALIV | 47-55 | 26 |
| (POL112) | | | |
| PD-L1 Fragment | SIGNAALQI | 93-101 | 27 |
| (POL113) | | | |
| PD-L1 Fragment | VILGAILLCL | 242-251 | 28 |
| (POL114) | | | |
| PD-L1 Fragment | HTAELVIPEL | 220-229 | 29 |
| (POL115) | | | |
| PD-L1 Fragment | FIFMTYWHLL | 7-16 | 30 |
| (POL116) | | | |
| PD-L1 Fragment | VIWTSSDHQV | 165-174 | 31 |
| (POL117) | | | |
| PD-L1 Fragment | FMTYWHLLNAFTVTVPKDL | 9-27 | 32 |
| (IO103)* | | | |

*Also referred to herein as PD-L1 long 1

In some embodiments, the PD-L1 immune checkpoint polypeptide fragment comprises or consists of the sequence of one of SEQ TD NOs: 15, 25, 28 or 32. In some embodiments, the PD-L1 immune checkpoint polypeptide fragment comprises or consists of the sequence of SEQ TD NO: 32.

Another preferred checkpoint for the purposes of the present invention is checkpoint (c), namely the interaction between the T cell receptor CTLA-4 and its ligands, the B7 proteins (B7-1 and B7-2). CTLA-4 is ordinarily upregulated on the T cell surface following initial activation, and ligand binding results in a signal which inhibits further/continued activation. CTLA-4 competes for binding to the B7 proteins with the receptor CD28, which is also expressed on the T cell surface, but which upregulates activation. Thus, by blocking the CTLA-4 interaction with the B7 proteins, but not the CD28 interaction with the B7 proteins, one of the normal check points of the immune response may be removed, leading to augmented anti-tumour T cell responses. Therefore, CTLA4 and its ligands are examples of immune checkpoints which may be targeted in the methods of the present disclosure.

In some embodiments, the methods provided herein comprise administering a polynucleotide encoding an immune checkpoint polypeptide or fragment thereof. In some embodiments, the polynucleotide is an RNA or a DNA polynucleotide.

### Immune Checkpoint Inhibitors

In some embodiments, the present disclosure provides methods for treating a cancer in a subject in need thereof comprising administering to the subject: (a) a first immune checkpoint peptide or a polynucleotide encoding the same; (b) a second immune checkpoint peptide or a polynucleotide encoding the same; and (c) an immune checkpoint inhibitor.

An “immune checkpoint inhibitor” is used herein to mean any agent which, when administered to a subject, blocks or inhibits the action of an immune checkpoint molecule, resulting in the upregulation of an immune effector response in the subject, typically a T cell effector response, which preferably comprises an anti-tumour T cell effector response.

The immune checkpoint inhibitor used in the method of the present invention may block or inhibit the action of any of the immune checkpoint molecules described above. The agent may be an antibody or any other suitable agent which results in said blocking or inhibition.

An “antibody” as used herein includes whole antibodies and any antigen binding fragment (i.e., “antigen-binding portion”) or single chains thereof. An antibody may be a polyclonal antibody or a monoclonal antibody and may be produced by any suitable method. Examples of binding fragments encompassed within the term “antigen-binding portion” of an antibody include a Fab fragment, a F(ab′)2 fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody.

In some embodiments, the antibody blocks or inhibits CTLA-4 interaction with B7 proteins. Examples of such antibodies include ipilumumab, tremelimumab, or any of the antibodies disclosed in WO2014/207063. Other molecules include polypeptides, or soluble mutant CD86 polypeptides.

In some embodiments, the antibody blocks or inhibits PD1 interaction with PD-L1 or PD-L2. In some embodiments, the antibody inhibits PD1. Examples of such anti-PD1 antibodies include Nivolumab, Pembrolizumab, Lambrolizumab, Pidilzumab, Cemiplimab, and AMP-224 (AstraZeneca/MedImmune and GlaxoSmithKline), JTX-4014 by Jounce Therapeutics, Spartalizumab (PDR001, Novartis), Camrelizumab (SHR1210, Jiangsu HengRui Medicine Co., Ltd), Sintilimab (IBI308, Innovent and Eli Lilly), Tislelizumab (BGB-A317), Toripalimab (JS 001), Dostarlimab (TSR-042, WBP-285, GlaxoSmithKline), INCMGA00012 (MGA012, Incyte and MacroGenics), and AMP-514 (MEDI0680, AstraZeneca). In some embodiments, the immune checkpoint inhibitor is an anti-PD-1 antibody selected from Nivolumab and Pembrolizumab. The immune checkpoint inhibitor is preferably Nivolumab or Pembrolizumab.

In some embodiments, the antibody inhibits PD-L1. Examples of such anti-PD-L1 antibodies include MEDI-4736, MPDL3280A, Atezolizumab (Tecentriq, Roche Genentech), Avelumab (Bavencio, Merck Serono and Pfizer), and Durvalumab (Imfinzi, AstraZeneca).

Other suitable inhibitors include small molecule inhibitors (SMI), which are typically small organic molecules.

Preferred inhibitors of IDO1 include Epacadostat (INCB24360), Indoximod, GDC-0919 (NLG919) and F001287. Other inhibitors of IDO1 include 1-methyltryptophan (1MT).

An immune checkpoint inhibitor of the invention, such as an antibody or SMI, may be formulated with a pharmaceutically acceptable excipient for administration to a subject. Suitable excipients and auxiliary substances are described below for the immunotherapeutic composition of the invention, and the same may also be used with the immune checkpoint inhibitor of the invention. Suitable forms for preparation, packaging and sale of the immunotherapeutic composition are also described above. The same considerations apply for the immune checkpoint inhibitor of the invention.

### Compositions

The disclosure provides compositions comprising the immune checkpoint polypeptides and/or immune checkpoint inhibitors as described herein. The compositions are typically pharmaceutical compositions. In some embodiments, the present disclosure provides a composition comprising: (a) a first immune checkpoint peptide or a polynucleotide encoding the same; (b) a second immune checkpoint peptide or a polynucleotide encoding the same; and (c) an immune checkpoint inhibitor.

In some embodiments, the first immune checkpoint peptide is formulated in a first composition, the second immune checkpoint peptide is formulated in a second composition, and the immune checkpoint inhibitor is formulated in a third composition. In some embodiments, the first and second immune checkpoint peptides are formulated in a first composition and the immune checkpoint inhibitor is formulated in a second composition. In some embodiments, the first immune checkpoint peptide, the second immune checkpoint peptide, and the immune checkpoint inhibitor are formulated together in one composition.

The compositions may comprise one immunogenic fragment of a component of an immune checkpoint molecule, or may comprise a combination of two or more such fragments, each interacting specifically with at least one different HLA molecule so as to cover a larger proportion of the target population. Thus, as examples, the composition may contain a combination of a peptide restricted by a HLA-A molecule and a peptide restricted by a HLA-B molecule, e.g. including those HLA-A and HLA-B molecules that correspond to the prevalence of HLA phenotypes in the target population, such as e.g. HLA-A2 and HLA-B35. Additionally, the composition may comprise a peptide restricted by an HLA-C molecule.

Compositions comprising one or more immune checkpoint polypeptides may preferably further comprise an adjuvant and/or a carrier. Adjuvants are any substance whose admixture into the composition increases or otherwise modifies the immune response elicited by the composition. Adjuvants, broadly defined, are substances which promote immune responses. Adjuvants may also preferably have a depot effect, in that they also result in a slow and sustained release of an active agent from the administration site. A general discussion of adjuvants is provided in Goding, Monoclonal Antibodies: Principles & Practice (2nd edition, 1986) at pages 61-63.

Adjuvants may be selected from the group consisting of: AlK(SO4)2, AlNa(SO4)2, AlNH4 (SO4), silica, alum, Al(OH)3, Ca3 (PO4)2, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-sn-glycero-3-hydroxphosphoryloxy)-ethylamine (CGP 19835A, also referred to as MTP-PE), RIBI (MPL+TDM+CWS) in a 2% squalene/Tween-80™ emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, Titermax, ISCOMS, Quil A, ALUN (see U.S. Pat. Nos. 58,767 and 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21. Various saponin extracts have also been suggested to be useful as adjuvants in immunogenic compositions. Granulocyte-macrophage colony stimulating factor (GM-CSF) may also be used as an adjuvant.

Preferred adjuvants to be used with the invention include oil/surfactant-based adjuvants such as Montanide adjuvants (available from Seppic, Belgium), preferably Montanide ISA-51. Other preferred adjuvants are bacterial DNA based adjuvants, such as adjuvants including CpG oligonucleotide sequences. Yet other preferred adjuvants are viral dsRNA based adjuvants, such as poly I:C. GM-CSF and Imidazochinilines are also examples of preferred adjuvants.

The adjuvant is most preferably a Montanide ISA adjuvant. The Montanide ISA adjuvant is preferably Montanide ISA 51 or Montanide ISA 720.

In Goding, Monoclonal Antibodies: Principles & Practice (2nd edition, 1986) at pages 61-63 it is also noted that, when an antigen of interest is of low molecular weight, or is poorly immunogenic, coupling to an immunogenic carrier is recommended. An immune checkpoint polypeptide or fragment described herein may be coupled to a carrier. A carrier may be present independently of an adjuvant. The function of a carrier can be, for example, to increase the molecular weight of a polypeptide fragment in order to increase activity or immunogenicity, to confer stability, to increase the biological activity, or to increase serum half-life. Furthermore, a carrier may aid in presenting the polypeptide or fragment thereof to T-cells. Thus, in the composition, the immune checkpoint polypeptide or fragment thereof may be associated with a carrier such as those set out below.

The carrier may be any suitable carrier known to a person skilled in the art, for example a protein or an antigen presenting cell, such as a dendritic cell (DC). Carrier proteins include keyhole limpet hemocyanin, serum proteins such as transferrin, bovine serum albumin, human serum albumin, thyroglobulin or ovalbumin, immunoglobulins, or hormones, such as insulin or palmitic acid. Alternatively, the carrier protein may be tetanus toxoid or diphtheria toxoid. Alternatively, the carrier may be a dextran such as sepharose. The carrier must be physiologically acceptable to humans and safe.

The compositions provided herein may optionally comprise a pharmaceutically acceptable excipient. The excipient must be ‘acceptable’ in the sense of being compatible with the other ingredients of the composition and not deleterious to the recipient thereof. Auxiliary substances, such as wetting or emulsifying agents, pH buffering substances and the like, may be present in the excipient. These excipients and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

The compositions provided herein may be prepared, packaged, or sold in a form suitable for bolus administration or for continuous administration. Injectable compositions may be prepared, packaged, or sold in unit dosage form, such as in ampoules or in multi-dose containers containing a preservative. Compositions include, but are not limited to, suspensions, solutions, emulsions in oily or aqueous vehicles, pastes, and implantable sustained-release or biodegradable formulations. In one embodiment of a composition, the active ingredient is provided in dry (for e.g., a powder or granules) form for reconstitution with a suitable vehicle (e. g., sterile pyrogen-free water) prior to administration of the reconstituted composition. The composition may be prepared, packaged, or sold in the form of a sterile injectable aqueous or oily suspension or solution. This suspension or solution may be formulated according to the known art, and may comprise, in addition to the active ingredient, additional ingredients such as the adjuvants, excipients and auxiliary substances described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. Alternatively, the active ingredients of the composition may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

### Exemplary Compositions

In some embodiments, the present disclosure provides a composition comprising (a) an IDO1 immune checkpoint polypeptide comprising or consisting of SEQ ID NO: 3, or polynucleotide encoding the same; (b) a PD-L1 immune checkpoint polypeptide comprising or consisting of SEQ ID NO: 32, or polynucleotide encoding the same; and (c) an anti-PD1 immune checkpoint inhibitor antibody. In such embodiments, the anti-PD1 immune checkpoint inhibitor antibody is nivolumab or pembrolizumab. In some embodiments, the composition further comprises an adjuvant.

In some embodiments, the present disclosure provides (i) a first composition comprising (a) an IDO1 immune checkpoint polypeptide comprising or consisting of SEQ ID NO: 3, or polynucleotide encoding the same and (b) a PD-L1 immune checkpoint polypeptide comprising or consisting of SEQ ID NO: 32, or polynucleotide encoding the same; and (ii) a second composition comprising nivolumab. In some embodiments, the first composition further comprises an adjuvant.

In some embodiments, the present disclosure provides (i) a first composition comprising an IDO1 immune checkpoint polypeptide comprising or consisting of SEQ ID NO: 3, or polynucleotide encoding the same; (ii) a second composition comprising a PD-L1 immune checkpoint polypeptide comprising or consisting of SEQ ID NO: 32, or polynucleotide encoding the same; and (iii) a third composition comprising nivolumab. In some embodiments, the first and/or second composition further comprises an adjuvant.

Said compositions may be provided for use in a method of the invention, or for use in any other method for the prevention or treatment of cancer which comprises administration of the compositions.

### Method for the Prevention or Treatment of Cancer

In some embodiments, the present disclosure provides methods for treating a cancer in a subject in need thereof comprising administering to the subject: (a) a first immune checkpoint peptide or a polynucleotide encoding the same; (b) a second immune checkpoint peptide or a polynucleotide encoding the same; and (c) an immune checkpoint inhibitor.

The cancer may be prostate cancer, brain cancer, breast cancer, colorectal cancer, pancreatic cancer, ovarian cancer, lung cancer, cervical cancer, liver cancer, head/neck/throat cancer, skin cancer, bladder cancer, or a hematologic cancer. In some embodiments, the cancer is in the form of a tumour or a blood born cancer. In some embodiments, the tumour is solid. In some embodiments, the tumour is malignant and may be metastatic. The tumour may be an adenoma, an adenocarcinoma, a blastoma, a carcinoma, a desmoid tumour, a desmopolastic small round cell tumour, an endocrine tumour, a germ cell tumour, a lymphoma, a leukaemia, a sarcoma, a Wilms tumour, a lung tumour, a colon tumour, a lymph tumour, a breast tumour, or a melanoma. In some embodiments, the melanoma is a metastatic melanoma.

Types of blastoma include hepatblastoma, glioblastoma, neuroblastoma or retinoblastoma. Types of carcinoma include colorectal carcinoma or heptacellular carcinoma, pancreatic, prostate, gastric, esophegal, cervical, and head and neck carcinomas, and adenocarcinoma. Types of sarcoma include Ewing sarcoma, osteosarcoma, rhabdomyosarcoma, or any other soft tissue sarcoma. Types of melanoma include Lentigo maligna, Lentigo maligna melanoma, Superficial spreading melanoma, Acral lentiginous melanoma, Mucosal melanoma, Nodular melanoma, Polypoid melanoma, Desmoplastic melanoma, Amelanotic melanoma, Soft-tissue melanoma, Melanoma with small nevus-like cells, Melanoma with features of a Spitz nevus and Uveal melanoma.

Types of lymphoma and leukaemia include Precursor T-cell leukemia/lymphoma, acute myeloid leukaemia, chronic myeloid leukaemia, acute lymphcytic leukaemia, Follicular lymphoma, Diffuse large B cell lymphoma, Mantle cell lymphoma, chronic lymphocytic leukemia/lymphoma, MALT lymphoma, Burkitt's lymphoma, Mycosis fungoides, Peripheral T-cell lymphoma, Nodular sclerosis form of Hodgkin lymphoma, Mixed-cellularity subtype of Hodgkin lymphoma. Types of lung tumour include tumours of non-small-cell lung cancer (adenocarcinoma, squamous-cell carcinoma and large-cell carcinoma) and small-cell lung carcinoma.

The method of the invention works by activating or augmenting the T cell anti-cancer response in a subject. This is achieved by increasing cancer or tumour-specific effector T cell activation, by blocking or inhibiting two or more immune checkpoints. The method of the invention utilises at least three different approaches to block or inhibit two or more immune checkpoints.

The first approach is to block or inhibit a first immune checkpoint by administering, typically in an immunotherapeutic composition, a first immune checkpoint polypeptide, an immunogenic fragment thereof, or polynucleotide encoding the same, which results in an immune response in the subject against the immune checkpoint molecule, thereby blocking or inhibiting the activity of the checkpoint. Thus, the immune checkpoint polypeptide or immunotherapeutic composition comprising same may alternatively be described as a vaccine against the immune checkpoint molecule. The immune checkpoint molecule which is targeted by the said immune response is preferably expressed by tumour cells and may also be expressed by normal cells which have an immune inhibitory effect. Accordingly, the immune response has a double effect in that it both blocks and inhibits the activity of the checkpoint and also directly attacks the tumour. In some embodiments, the first immune checkpoint polypeptide is an IDO1 polypeptide, fragment thereof, or polynucleotide encoding the same. Therefore, in some embodiments, the methods provided herein comprise administering an immunotherapeutic composition comprising an immunogenic fragment of IDO1 or a polynucleotide encoding the same.

The second approach is to block or inhibit a second, different immune checkpoint by administering, typically in an immunotherapeutic composition, a second immune checkpoint polypeptide, an immunogenic fragment thereof, or a polynucleotide encoding the same, which results in an immune response in the subject against the second immune checkpoint molecule, thereby blocking or inhibiting the activity of the second immune checkpoint. Thus, the immune checkpoint polypeptide, immunogenic fragment thereof, or a polynucleotide encoding the same, or immunotherapeutic composition comprising same may alternatively be described as a vaccine against the second immune checkpoint molecule. The second immune checkpoint molecule is preferably expressed by tumour cells and may also be expressed by normal cells which have an immune inhibitory effect. Accordingly, the said immune response has a double effect in that it both blocks and inhibits the activity of the second immune checkpoint molecule and also directly attacks the tumour. In some embodiments, the second immune checkpoint polypeptide is a PD-L1 polypeptide. Therefore, in some embodiments, the methods provided herein comprise administering an immunotherapeutic composition comprising an immunogenic fragment of PD-L1 or a polynucleotide encoding the same.

The third approach is to block or inhibit an immune checkpoint by administering an immune checkpoint inhibitor which binds to or otherwise modifies an immune checkpoint molecule, thereby blocking or inhibiting the activity of the immune checkpoint molecule. The agent may be an antibody or small molecule inhibitor which binds to an immune checkpoint molecule. Multiple such agents may be administered, each of which targets a different immune checkpoint molecule. In some embodiments, the immune checkpoint inhibitor targets PD1. In some embodiments, the immune checkpoint inhibitor is an antibody which specifically binds to PD1.

Methods of the present disclosure comprise administration of an immunotherapeutic composition comprising an IDO1 immune checkpoint polypeptide, administration of an immunotherapeutic composition comprising a PD-L1 immune checkpoint polypeptide, and administration of an immune checkpoint inhibitor which interferes with PD1 binding to PD-L1 and/or PD-L, for the treatment or prevention of cancer. In particular embodiments, the cancer is metastatic melanoma.

The IDO1 immune checkpoint polypeptide and PD-L1 immune checkpoint polypeptide may be administered in separate or in the same, single immunotherapeutic composition. The IDO1 immune checkpoint polypeptide and PD-L1 immune checkpoint polypeptide may be administered via administration of a nucleic acid encoding the amino acid sequence of the component or fragment. Preferred components and fragments of the immune system checkpoints, and immunotherapeutic compositions comprising said components and fragments are discussed in the preceding section. Preferred immunomodulatory agents are discussed in the relevant section above. However, a particularly preferred embodiment of the invention is a method for the prevention or treatment of cancer, particularly metastatic melanoma in a subject, the method comprising administering to said subject:
(a) a polypeptide fragment of IDO of up to 50 amino acids in length which comprises or consists of the amino acid sequence of SEQ ID NO: 3
(b) a polypeptide fragment of PD-L1 of up to 50 amino acids in length which comprises or consists of the amino acid sequence of SEQ ID NO: 32
(c) an anti-PD1 antibody, such as pembrolizumab or nivolumab.

By exploiting different approaches to block or inhibit immune system checkpoints, the method of the invention will result in a greater anti-tumour response with fewer side-effects or complications as compared to alternative methods. The anti-tumour response is typically greater than that which would be expected if only a single approach were used. In addition, there are less likely to be reductions in efficacy due to anti-drug responses, since the first and second approaches (the vaccines) will actively benefit from such a response, which may also result in a long-lasting effect. These benefits may be enhanced even if the third approach targets the same immune system checkpoint as one of the first or second approaches.

In some embodiments, the subject treated by the methods provided herein have not previously received treatment with an immune checkpoint inhibitor. For example, in some embodiments, the method comprises administering to the subject: (a) a first immune checkpoint peptide or a polynucleotide encoding the same; (b) a second immune checkpoint peptide or a polynucleotide encoding the same; and (c) an immune checkpoint inhibitor of PD1, wherein the subject has not previously received treatment with an immune checkpoint inhibitor of PD1.

In some embodiments, the subject has previously received treatment with an immune checkpoint inhibitor. In some such embodiments, the subject was refractory to treatment with the immune checkpoint inhibitor (i.e., the subject did not respond to the immune checkpoint inhibitor). In some such embodiments, the subject developed resistance to treatment with the immune checkpoint inhibitor (i.e., the subject initially responded treatment with immune checkpoint inhibitor and later became resistant to such treatment).

In some embodiments, the present disclosure provides methods for preventing disease progression in a subject suffering from a cancer comprising administering to the subject: (a) a first immune checkpoint peptide or a polynucleotide encoding the same; (b) a second immune checkpoint peptide or a polynucleotide encoding the same; and (c) an immune checkpoint inhibitor. In such embodiments, the cancer does not progress for at least 1, 2, 3, 4, 5, 6, 7, 8, 9, 10, 11, 12, 13, 14, 15, or more years after completion of treatment.

In some embodiments, the present disclosure provides methods for reducing tumour volume and/or number in a subject suffering from a cancer comprising administering to the subject: (a) a first immune checkpoint peptide or a polynucleotide encoding the same; (b) a second immune checkpoint peptide or a polynucleotide encoding the same; and (c) an immune checkpoint inhibitor. In some such embodiments, the tumour volume and/or tumour number is reduced by 20%, 30%, 40%, 50%, 60%, 70%, 80%, 90%, or 100%.

### Administration Regimen

In order to treat cancer, immunotherapeutic compositions comprising immune checkpoint polypeptides and/or immune checkpoint inhibitors are each administered to the subject in a therapeutically effective amount. By a “therapeutically effective amount” of a substance, it is meant that a given substance is administered to a subject suffering from cancer, in an amount sufficient to cure, alleviate or partially arrest the cancer or one or more of its symptoms. Such therapeutic treatment may result in a decrease in severity of disease symptoms, or an increase in frequency or duration of symptom-free periods. Such treatment may result in a reduction in the volume of a solid tumour.

In order to prevent cancer, immunotherapeutic compositions comprising immune checkpoint polypeptides and/or immune checkpoint inhibitors are each administered to the subject in a prophylactically effective amount. By “prophylactically effective amount” of a substance, it is meant that a given substance is administered to a subject in an amount sufficient to prevent occurrence or recurrence of one or more of symptoms associated with cancer for an extended period.

Effective amounts for a given purpose and a given composition or agent will depend on the severity of the disease as well as the weight and general state of the subject and may be readily determined by the physician.

Immunotherapeutic compositions comprising immune checkpoint polypeptides and/or immune checkpoint inhibitors may be administered simultaneously or sequentially, in any order. The appropriate administration routes and doses for each may be determined by a physician, and the composition and agent formulated accordingly.

In some embodiments, immunotherapeutic compositions comprising immune checkpoint polypeptides and/or immune checkpoint inhibitors are administered via a parenteral route, typically by injection. Administration may preferably be via a subcutaneous, intradermal, intramuscular, or intratumoral route. The injection site may be pre-treated, for example with imiquimod or a similar topical adjuvant to enhance immunogenicity. The total amount of each individual polypeptide present as active agent in a single dose of an immunotherapeutic composition will typically be in the range of 10 μg to 1000 μg, preferably 10 μg to 200 μg, preferably around 100 μg of each polypeptide present as active agent. For example, if there are two polypeptide active agents, and each is present at around 100 μg, the total quantity of peptide will be around 200 μg.

When the immune checkpoint inhibitor is an antibody, it is typically administered as a systemic infusion, for example intravenously. When the immune checkpoint inhibitor is an SMI it is typically administered orally. Appropriate doses for antibodies and SMIs may be determined by a physician. Appropriate doses for antibodies are typically proportionate to the body weight of the subject.

A typical regimen for the method of the invention will involve multiple, independent administrations of one or more immunotherapeutic compositions comprising immune checkpoint polypeptides and/or immune checkpoint inhibitors. Each may be independently administered on more than one occasion, such as two, three, four, five, six, seven, ten, fifteen, twenty or more times. An immunotherapeutic composition in particular may provide an increased benefit if it is administered on more than one occasion, since repeat doses may boost the resulting immune response. A typical regimen may include up to 15 series of administration of the immunotherapeutic compositions. Individual administrations of compositions or agents may be separated by an appropriate interval determined by a physician. The interval between administrations will typically be shorter at the beginning of a course of treatment and will increase towards the end of a course of treatment. For example, a composition may be administered biweekly, for up to around 6 weeks, and then once every four weeks for around a further 9 rounds.

An exemplary administration regimen comprises administration of an immune checkpoint inhibitor at, for example a dose of 3 milligram per kilogram of body weight biweekly, for a total of up to around 24 series, with an immunotherapeutic composition comprising one or more immune checkpoint polypeptides (typically including an adjuvant) also administered subcutaneously on the back of the arm or front of the thigh, alternating between the right and the left side. This may be administered biweekly, for up to around 6 weeks, and then once every four weeks for a further 9 rounds. Administration of the immunotherapeutic composition may be initiated concomitantly with the first series of agent or may be initiated later. For example, such that the final round of immunotherapeutic composition administration occurs concomitantly with the final round agent.

### Immune Profiles

In some embodiments, the present disclosure provides methods of treating cancer in a patient in need thereof, wherein the patient has an immune profile indicative of response to treatment with an IDO1 polypeptide, a PD-L1 polypeptide, and an antibody that binds to PD1.

In some embodiments, the present disclosure provides a method for stratifying a cancer patient into one of at least two treatment groups, wherein said method comprises: analysing one or more cell populations in a peripheral blood sample from the patient to determine an immune profile; and: (i) stratifying the patient into a first treatment group if the immune profile is indicative of response to treatment with an IDO1 polypeptide, a PD-L1 polypeptide and an antibody that binds to PD1; or (ii) stratifying the patient into a second treatment group if the immune profile determined in step indicates that the subject will not respond to said treatment. In such embodiments, the first treatment group is treated with the IDO1 polypeptide, the PD-L1 polypeptide and the antibody that binds to PD1 and the second treatment group is to be treated with one or more alternative therapies (e.g., chemotherapy, radiation, and/or additional immunotherapies).

As used herein, “immune profile” refers to a collection of one or more biomarkers used to describe an immune response or state of a patient at a particular moment in time (e.g., a snapshot of a baseline immune state or a snapshot of an ongoing immune response to some sort of stimulus). The biomarkers comprising an immune profile may be selected from a variety of markers including gene expression, protein expression, metabolite levels, and/or cell populations.

In some embodiments, the immune profiles described herein are comprised of an analysis of one or more cell populations. In such embodiments, a sample from a patient is analysed by flow cytometry to determine the percentage and/or number of a particular cell type present in the sample based on cell-surface marker expression and/or intracellular protein expression. In some embodiments, the sample is a peripheral blood sample. In some embodiments, the sample is a tissue sample, such as a tumor sample.

In some embodiments, the immune profiles described herein are determined by an analysis of a peripheral blood sample from a patient. In some embodiments, peripheral blood monocytes are isolated from the peripheral blood sample and used as the starting material for analysis by flow cytometry. In some embodiments, the percentage and/or total number of one or more of the following cell types is determined: T regulatory cells, activated T cells (e.g., LAG3+ and/or CD28+ T cells, monocytic-myeloid derived suppressor cells (mMDSCs), natural killer (NK) cells, and/or dendritic cells. The determined percentage and/or total number of one or more cell type thus comprises the immune profile of the patient for the time point at which the sample was collected. In some embodiments, the immune profile of a patient is used to predict responsiveness to treatment with the compositions described herein.

In some embodiments, the immune profile of patient as determined herein is compared to that of a control subject group. A control subject group refers to a group of subjects that do not suffer from a disease to be treated by the methods described herein (i.e., does not suffer from a cancer). Immune profiles of control subject groups can be determined from historical controls.

In some embodiments, the immune profile is a baseline immune profile wherein the immune profile is determined in a patient prior to the patient receiving treatment with a composition described herein. In some embodiments, the immune profile is determined at one or more time points after the subject has received treatment with a composition described herein.

In some embodiments, an immune profile comprises an analysis of T cell populations in a peripheral blood sample. T cells are defined herein as lymphocyte that expresses a T cell receptor (TCR) and CD3 (TCR+CD3+). T cells can be further subdivided into many groups including T helper cells (TCR+CD3+CD4+), cytolytic T cells (TCR+CD3+CD8+), T regulatory cells, and others.

In some embodiments, an immune profile comprises an analysis of T regulatory cells in a peripheral blood sample. Herein, a T regulatory (Treg) cell is defined as a lymphocyte that expresses TCR*CD3+CD25^{High}CD127^{Low}. In some embodiments, Tregs are further defined by intracellular FoxP3 expression. In some embodiments, a decrease in T regulatory cells (as a percentage of CD4+ T cells) compared to a control subject group is indicative of a likelihood of response to treatment with a composition described herein. In some embodiments, a percentage of T regulatory cells (as a percentage of CD4+ T cells) that is less than 7% is indicative of a likelihood of response to treatment with a composition described herein. In some embodiments, a percentage of T regulatory cells (as a percentage of CD4+ T cells) that is less than 6.5%, less than 6%, less than 5.5%, less than 5%, less than 4.5%, less than 4%, less than 3.5%, or less than 3% is indicative of a likelihood of response to treatment a composition described herein.

In some embodiments, an immune profile comprises an analysis of an activated T cells in a peripheral blood sample. In some embodiments, the activated T cells express CD28+CD4+ T cells. In some embodiments, a decrease in CD28+CD4+ T cells (as a percentage of CD4+ T cells) compared to a control subject group is indicative of a likelihood of response to treatment with a composition described herein. In some embodiments, a percentage of CD28+CD4+ T cells (as a percentage of CD4+ T cells) that is less than 70% is indicative of a likelihood of response to treatment with a composition described herein. In some embodiments, a percentage of CD28+CD4+ T cells (as a percentage of CD4+ T cells) that is less than 65%, less than 60%, less than 55%, less than 50%, less than 45%, less than 40%, less than 35%, less than 30%, less than 25%, less than 20%, less than 15%, less than 10%, or less than 5% is indicative of a likelihood of response to treatment with a composition described herein.

In some embodiments, the activated T cells express LAG3+CD4*. In some embodiments, an immune profile comprises an analysis of LAG3+CD4+ T cells in a peripheral blood sample. In some embodiments, an increase in LAG3+CD4+ T cells (as a percentage of CD4+ T cells) compared to a control subject group is indicative of a likelihood of response to treatment with a composition described herein. In some embodiments, a percentage of LAG3+CD4+ T cells (as a percentage of CD4+ T cells) that is 12% or more is indicative of a likelihood of response to treatment with a composition described herein. In some embodiments, a percentage of LAG3+CD4+ T cells (as a percentage of CD4+ T cells) that is 15% or more, 20% or more, 25% or more, 30% or more, 35% or more, 40% or more, 45% or more, 50% or more, 55% or more, 60% or more, 65% or more, 70% or more, 75% or more, or 80% or more is indicative of a likelihood of response to treatment with a composition described herein.

In some embodiments, an immune profile comprises an analysis of monocytic-myeloid derived suppressor cells (mMDSCs) in a peripheral blood sample. Herein, mMDSCs are myeloid cells expressing the following cell surface marker panel: CD3-CD19-CD56-HLADR-CD14+CD33+. In some embodiments, a decrease in mMDSCs (as a percentage of total PBMCs) compared to a control subject group is indicative of a likelihood of response to treatment with a composition described herein. In some embodiments, a percentage of mMDSCs (as a percentage of total PBMCs) that is less than 10% is indicative of a likelihood of response to treatment with a composition described herein. In some embodiments, a percentage of mMDSCs (as a percentage of total PBMCs) that is less than 9.5%, less than 9%, less than 8.5%, less than 8%, less than 7.5%, less than 7%, less than 6.5%, less than 6%, less than 5.5.%, less than 5%, less than 4.5%, less than 4%, less than 3.5%, or less than 3% is indicative of a likelihood of response to treatment with a composition described herein.

In some embodiments, an immune profile comprises an analysis of natural killer (NK) cells in a peripheral blood sample. In some embodiments, the NK cells are CD56 m^{m}CD16+NK cells. In some embodiments, the NK cells are CD56^{bright}CD16− NK cells. CD56 m^{m} NK cells are the main NK cell population found in peripheral blood and are known to be cytotoxic and immunostimulatory. CD56^{bright} NK cells are a rarer NK cell population in the blood but are abundant in certain tissues and are implicated in immune modulation. In some embodiments, an increase in CD56^{dim}CD16+NK cells compared to a control subject group is indicative of a likelihood of response to treatment a composition described herein. In some embodiments, a percentage of CD56 m^{m}CD16+NK cells (as a percentage of PBMCs) that is 6% or more is indicative of a likelihood of response to treatment with a composition described herein. In some embodiments, a percentage of CD56 m^{m}CD16+NK cells (as a percentage of PBMCs) that is 7%, 8%, 9%, 10%, 11%, 12%, 13%, 14%, 15%, 16%, 17%, 18%, 19%, or 20% or more is indicative of a likelihood of response to treatment with a composition described herein. In some embodiments, a decrease in CD56^{bright}CD16− NK cells compared to a control subject group is indicative of a likelihood of response to treatment with a composition described herein. In some embodiments, a percentage of CD56^{bright}CD16− NK cells (as a percentage of PBMCs) that is less than 0.3% is indicative of a likelihood of response to treatment with a composition described herein. In some embodiments, a percentage of CD56^{bright}CD16− NK cells (as a percentage of PBMCs) that is less than 0.25%, less than 0.2%, less than 0.15%, or less than 0.1% is indicative of a likelihood of response to treatment with a composition described herein.

In some embodiments, an immune profile comprises an analysis of conventional dendritic cells type 2 (cDC2) in a peripheral blood sample. cDC2 are a dendritic cell subset with various roles in inflammatory processes (See Collin M, Bigley V. Human dendritic cell subsets: an update. Immunology. 2018; 154(1):3-20. doi:10.1111/imm. 12888). Herein, cDC2 cells express the following cell surface markers: CD3-CD19-CD56-CD11c+CD16-CD14-CD33+CD1c+. In some embodiments, an increase in cDC2s (as a percentage of total PBMCs) compared to a control subject group is indicative of a likelihood of response to treatment with a composition described herein. In some embodiments, a percentage of cDC2s (as a percentage of total PBMCs) that is 0.01% or more is indicative of a likelihood of response to treatment with a composition described herein. In some embodiments, a percentage of cDC2s (as a percentage of total PBMCs) that is 0.02% or more, 0.03% or more, 0.04% or more, 0.05% or more, 0.06% or more, 0.07% or more, 0.08% or more, 0.09% or more, 0.10% or more, 0.11% or more, 0.12% or more, 0.13% or more, 0.14% or more, 0.15% or more, 0.16% or more, 0.17% or more, 0.18% or more, 0.19% or more, 0.20% or more, 0.30% or more, 0.40% or more, 0.50% or more, 0.60% or more, 0.70% or more, 0.80% or more, 0.90% or more, or 1% or more is indicative of a likelihood of response to treatment with a composition described herein.

In some embodiments, the immune profile indicative of a likelihood of response to treatment with a composition described herein comprises one or more of the following: a) less than 6% (e.g., less than 6%, less than 5%, less than 4%, less than 3%, or less than 2%) CD4+ T regulatory cells as a percentage of CD4+ T cells; b) less than 70% (e.g., less than 60%, less than 50%, less than 40%, less than 30%, or less than 20%) CD28+CD4+ T cells as a percentage of CD4+ T cells; c) greater than 12% (e.g., greater than 12%, greater than 15%, greater than 20%, greater than 25%, greater than 30%, greater than 35%, greater than 40%, or more) LAG-3+CD4+ T cells as a percentage of CD4+ T cells; d) less than 10% (e.g., less than 10%, less than 9%, less than 8%, less than 7%, less than 6%, less than 5%, less than 4%, less than 3%, or less than 2%) monocytic-myeloid derived suppressor cells (mMDSCs) as a percentage of PBMCs; e) greater than 6% (e.g., greater than 6%, greater than 7%, greater than 8%, greater than 9%, greater than 10%, greater than 15%, or more) CD56^{dim}CD16+ Natural Killer (NK) cells as a percentage of PBMCs; f) less than 0.3% (e.g., less than 0.3%, less than 0.2%, less than 0.1%, or less than 0.5%) CD56^{bright}CD16− NK cells as a percentage of PBMCs; and/or g) greater than 0.01% (e.g., greater than 0.01%, greater than 0.05%, greater than 0.1%, greater than 0.2%, greater than 0.3%, greater than 0.4%, greater than 0.5%) conventional dendritic cells type 2 (cDC2) as a percentage of PBMCs.

In some embodiments, the present disclosure provides a method of monitoring the response of a cancer patient to treatment with an IDO1 polypeptide, a PD-L1 polypeptide and an antibody that binds to PD1, wherein said method comprises: analysing one or more cell populations in a peripheral blood sample from the subject to determine an immune profile; and (i) determining that the patient is responding to treatment if the patient has an immune profile indicative of response to treatment; or (ii) determining that the patient is not responding to treatment if the patient does not have an immune profile indicative of response to treatment. In such embodiments, peripheral blood is taken from a patient prior to treatment and at one or more time points after treatment has commenced. An immune profile is determined from the peripheral blood sample taken prior to treatment (i.e., the baseline immune profile) and a second immune profile is determined from one or more peripheral blood samples taken after treatment has commenced. The changes in various biomarkers (e.g., changes in cell populations and/or cell surface marker expression) are assess between the two immune profiles to determine whether the patient is responding to the treatment. If a patient is responding to treatment, treatment is continued according to the original treatment protocol. If a patient is not responding, one or more adjustments to the treatment protocol may be made (e.g., a change in treatment dose and/or frequency), or treatment may be terminated.

In some embodiments, an immune profile indicative of a patient's response to treatment comprises an increased percentage of CD28+, HLA-DR+, CD39+, TIGIT+ and/or TIM-3+CD4+ T cells compared to the percentage of these same CD4+ T cell subsets determined in the baseline immune profile. In some embodiments, an immune profile indicative of a patient's response to treatment comprises an increased percentage of HLA-DR+, CD39+, LAG-3+ and/or TIGIT+CD8+ T-cells compared to the percentage of these same CD8+ T cell subsets determined in the baseline immune profile. In some embodiments, an immune profile indicative of a patient's response to treatment comprises an increased percentage of CD28+, HLA-DR+, CD39+, TIGIT+ and/or TIM-3+CD4+ T cells and an increased percentage of HLA-DR+, CD39+, LAG-3+ and/or TIGIT+CD8+ T-cells. In some embodiments, an immune profile indicative of a patient's response to treatment comprises an increased percentage of CD28+, HLA-DR+, CD39+, TIGIT+ and/or TIM-3+CD4+ T cells of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least, at least 250%, at least 300%, at least 350%, at least 400%, at least 450%, or at least 500%. In some embodiments, an immune profile indicative of a patient's response to treatment comprises an increased percentage of HLA-DR+, CD39+, LAG-3+ and/or TIGIT+CD8+ T-cells of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least, at least 250%, at least 300%, at least 350%, at least 400%, at least 450%, or at least 500%.

### Further Numbered Embodiments

Embodiment 1. A method for treating a cancer in a subject in need thereof comprising administering to the subject: a first immune checkpoint polypeptide or a polynucleotide encoding the same; a second immune checkpoint polypeptide or a polynucleotide encoding the same; and an immune checkpoint inhibitor.

Embodiment 2. A method of preventing disease progression in a subject suffering from a cancer comprising administering to the subject: a first immune checkpoint polypeptide or a polynucleotide encoding the same; a second immune checkpoint polypeptide or a polynucleotide encoding the same; and an immune checkpoint inhibitor.

Embodiment 3. A method of reducing tumor volume in a subject suffering from a cancer comprising administering to the subject: a first immune checkpoint polypeptide or a polynucleotide encoding the same; a second immune checkpoint polypeptide or a polynucleotide encoding the same; and an immune checkpoint inhibitor.

Embodiment 4. The method of any one of Embodiments 1-3, wherein the subject has not previously received treatment with the immune checkpoint inhibitor.

Embodiment 5. The method of any one of Embodiments 1-3, wherein the subject has previously received treatment with the immune checkpoint inhibitor.

Embodiment 6. The method of Embodiment 5, wherein the subject was refractory to the treatment with the immune checkpoint inhibitor or developed resistance to the immune checkpoint inhibitor during the course of the previous treatment.

Embodiment 7. The method of any one of Embodiments 1-6, wherein the first and second immune checkpoint polypeptide are independently selected from an IDO1 peptide, a PD-1 peptide, a PD-L1 peptide, a PD-L2 peptide, a CTLA4 peptide, a B7-H3 peptide, a B7-H4 peptide, an HVEM peptide, a BTLA peptide, a GAL9 peptide, a TIM3 peptide, a LAG3 peptide, or a KIR polypeptide.

Embodiment 8. The method of any one of Embodiments 1-7, wherein the first immune checkpoint polypeptide is an IDO1 polypeptide and wherein the second immune checkpoint polypeptide is a PD-L1 polypeptide.

Embodiment 9. The method of Embodiment 8, wherein the IDO1 polypeptide consists of up to 50 consecutive amino acids of SEQ ID NO: 1, and wherein said consecutive amino acids comprise the sequence of ALLEIASCL (SEQ ID NO: 2) or the sequence of DTLLKALLEIASCLEKALQVF (SEQ ID NO: 3).

Embodiment 10. The method of Embodiment 8 or 9, wherein the IDO1 polypeptide comprises or consists of the sequence of ALLEIASCL (SEQ ID NO: 2) or the sequence of DTLLKALLEIASCLEKALQVF (SEQ ID NO: 3).

Embodiment 11. The method of Embodiment 8, wherein the PD-L1 polypeptide consists of up to 50 consecutive amino acids of SEQ ID NO: 14, and wherein said consecutive amino acids comprise the sequence of any one of SEQ ID NOs: 15 to 32.

Embodiment 12. The method of Embodiment 8, wherein the PD-L1 polypeptide consists of up to 50 consecutive amino acids of SEQ ID NO: 14, and wherein said consecutive amino acids comprise the sequence of any one of SEQ ID NOs: 15, 25, 28 or 32.

Embodiment 13. The method of Embodiment 8, wherein the PD-L1 peptide comprises or consists of the sequence of FMTYWHLLNAFTVTVPKDL (SEQ ID NO: 32).

Embodiment 14. The method of Embodiment 8, wherein the IDO1 polypeptide comprises or consists of the sequence of ALLEIASCL (SEQ ID NO: 2) or the sequence of DTLLKALLEIASCLEKALQVF (SEQ ID NO: 3) and the PD-L1 peptide comprises or consists of the sequence of FMTYWHLLNAFTVTVPKDL (SEQ ID NO: 32).

Embodiment 15. The method of any one of Embodiments 1-14, wherein the immune checkpoint inhibitor is an antibody or small molecule inhibitor (SMI).

Embodiment 16. The method of Embodiment 15, wherein the SMI is an inhibitor of IDO1.

Embodiment 17. The method of Embodiment 16, wherein the SMI is selected from Epacadostat (INCB24360), Indoximod, GDC-0919 (NLG919), and F001287.

Embodiment 18. The method of Embodiment 15, wherein the antibody binds to CTLA4 or PD1.

Embodiment 19. The method of Embodiment 18, wherein the antibody that binds to CTLA4 is ipilimumab.

Embodiment 20. The method of Embodiment 18, wherein the antibody that binds to PD-1 is pembrolizumab or nivolumab.

Embodiment 21. The method of any one of Embodiments 1-20, wherein (a) and (b) are administered as a first composition and (c) is administered as a second composition.

Embodiment 22. The method of any one of Embodiments 1-20, wherein (a), (b), and (c) are administered as one composition.

Embodiment 23. The method of Embodiment 21 or 22, wherein the compositions further comprise an adjuvant or carrier.

Embodiment 24. The method of Embodiment 23, wherein the adjuvant is selected from herein said adjuvant is a Montanide ISA adjuvant, a bacterial DNA adjuvant, an oil/surfactant adjuvant, a viral dsRNA adjuvant, an imidazoquinoline, and GM-CSF.

Embodiment 25. The method of Embodiment 24, wherein the Montanide ISA adjuvant is selected from Montanide ISA 51 and Montanide ISA 720.

Embodiment 26. The method of any one of Embodiments 3-25, wherein the disease does not progress for at least 12 months, at least 2 years, at least 3 years, at least 4 years, at least 5 years, or longer after completion of treatment.

Embodiment 27. The method of any one of Embodiments 1-26, wherein the cancer is selected from prostate cancer, brain cancer, breast cancer, colorectal cancer, pancreatic cancer, ovarian cancer, lung cancer, cervical cancer, liver cancer, head/neck/throat cancer, skin cancer, bladder cancer, or a hematologic cancer.

Embodiment 28. The method of any one of Embodiments 1-26, wherein the cancer is a solid tumor cancer selected from an adenoma, an adenocarcinoma, a blastoma, a carcinoma, a desmoid tumour, a desmopolastic small round cell tumour, an endocrine tumour, a germ cell tumour, a lymphoma, a leukaemia, a sarcoma, a Wilms tumour, a lung tumour, a colon tumour, a lymph tumour, a breast tumour, or a melanoma.

Embodiment 29. The method of any one of Embodiments 1-26, wherein the cancer is metastatic melanoma.

Embodiment 30. The method of any one of Embodiments 1-29, wherein the subject has an immune profile indicative of response to treatment with the first immune checkpoint polypeptide or the polynucleotide encoding the same, the second immune checkpoint polypeptide or the polynucleotide encoding the same, and the immune checkpoint inhibitor.

Embodiment 31. A method for treating a cancer in a subject in need thereof comprising administering to the subject: an IDO immune checkpoint polypeptide or a polynucleotide encoding the same, wherein the IDO polypeptide comprises or consists of the sequence of ALLEIASCL (SEQ ID NO: 2) or the sequence of DTLLKALLEIASCLEKALQVF (SEQ ID NO: 3); a PD-L1immune checkpoint polypeptide or a polynucleotide encoding the same, wherein the PD-L1 polypeptide comprises or consists of the sequence of FMTYWHLLNAFTVTVPKDL (SEQ ID NO: 32); and an immune checkpoint inhibitor, wherein the immune checkpoint inhibitor is an anti-PD1 antibody.

Embodiment 32. A method of preventing disease progression in a subject suffering from a cancer comprising administering to the subject: an IDO immune checkpoint polypeptide or a polynucleotide encoding the same, wherein the IDO polypeptide comprises or consists of the sequence of ALLEIASCL (SEQ ID NO: 2) or the sequence of DTLLKALLEIASCLEKALQVF (SEQ ID NO: 3); a PD-L1immune checkpoint polypeptide or a polynucleotide encoding the same, wherein the PD-L1 polypeptide comprises or consists of the sequence of FMTYWHLLNAFTVTVPKDL (SEQ ID NO: 32); and an immune checkpoint inhibitor, wherein the immune checkpoint inhibitor is an anti-PD1 antibody.

Embodiment 33. A method of reducing tumor volume in a subject suffering from a cancer comprising administering to the subject: an IDO immune checkpoint polypeptide or a polynucleotide encoding the same, wherein the IDO polypeptide comprises or consists of the sequence of ALLEIASCL (SEQ ID NO: 2) or the sequence of DTLLKALLEIASCLEKALQVF (SEQ ID NO: 3); a PD-L1 immune checkpoint polypeptide or a polynucleotide encoding the same, wherein the PD-L1 polypeptide comprises or consists of the sequence of FMTYWHLLNAFTVTVPKDL (SEQ ID NO: 32); and an immune checkpoint inhibitor, wherein the immune checkpoint inhibitor is an anti-PD1 antibody.

Embodiment 34. The method of any one of Embodiments 30-33, wherein the subject has an immune profile indicative of response to treatment with the IDO polypeptide or polynucleotide encoding the same, the PD-L1 polypeptide or polynucleotide encoding the same, and the anti-PD1 antibody.

Embodiment 35. A kit comprising: a first immune checkpoint polypeptide or a polynucleotide encoding the same; a second immune checkpoint polypeptide or a polynucleotide encoding the same; and an immune checkpoint inhibitor.

Embodiment 36. The kit of Embodiment 35, wherein (a) and (b) are provided as a single composition, in a separate sealed container from (c).

Embodiment 37. An immunotherapeutic composition for use in a method for the prevention or treatment of cancer in a subject, wherein the immunotherapeutic composition comprises (a) and/or (b) as defined in Embodiment 1, and wherein the method is as defined in Embodiment 1.

Embodiment 38. Use of an immunotherapeutic composition in the manufacture of a medicament for the prevention or treatment of cancer in a subject, the immunotherapeutic composition comprising (a) and/or (b) as defined in Embodiment 1, which is formulated for administration before, concurrently with, and/or after an immune checkpoint inhibitor.

Embodiment 39. An immunotherapeutic composition for use according to Embodiment 37, or the use according to Embodiment 38, wherein the subject has an immune profile indicative of response to treatment with the first immune checkpoint polypeptide or the polynucleotide encoding the same, the second immune checkpoint polypeptide or the polynucleotide encoding the same, and the immune checkpoint inhibitor.

Embodiment 40. The immunotherapeutic composition for use according to Embodiment 39 or the use according to Embodiment 39, wherein the first immune polypeptide is an IDO1 polypeptide, the second immune polypeptide is a PD-L1 polypeptide and the immune checkpoint inhibitor is an antibody that binds to PD1.

Embodiment 41. The method of Embodiment 30 or 34, or the immunotherapeutic composition for use according to Embodiment 39 or 40, or the use according to Embodiment 39 or 40, wherein the immune profile comprises one or more of the following: a) a decrease in CD4+ T regulatory cells compared to a control subject group; b) a decrease in CD28+CD4+ T cells compared to a control subject group; c) an increase in LAG-3+CD4+ T cells compared to a control subject group; d) a decrease in monocytic-myeloid derived suppressor cells (mMDSCs) compared to a control subject group; e) an increase in CD56dimCD16+ Natural Killer (NK) cells compared to a control subject group; f) a decrease in CD56^{bright}CD16− NK cells compared to a control subject group; and/or g) an increase in conventional dendritic cells type 2 (cDC2) compared to a control subject group.

Embodiment 41A. The method of Embodiment 30 or 34, or the immunotherapeutic composition for use according to Embodiment 39 or 40, or the use according to Embodiment 39 or 40, wherein the immune profile comprises one or more of the following: a) less than 6% CD4+ T regulatory cells as a percentage of CD4+ T cells; b) less than 70% CD28+CD4+ T cells as a percentage of CD4+ T cells; c) greater than 12% LAG-3+CD4+ T cells as a percentage of CD4+ T cells; d) less than 10% monocytic-myeloid derived suppressor cells (mMDSCs) as a percentage of PBMCs; e) greater than 6% CD56^{dim}CD16+ Natural Killer (NK) cells as a percentage of PBMCs; f) less than 0.3% CD56^{bight}CD16− NK cells as a percentage of PBMCs; and/or g) greater than 0.01% conventional dendritic cells type 2 (cDC2) as a percentage of PBMCs.

Embodiment 42. The method, the immunotherapeutic composition, or the use of Embodiment 41, wherein the cell populations are determined by FACS analysis of a peripheral blood sample obtained from the subject.

Embodiment 43. A method for stratifying a cancer patient into one of at least two treatment groups, wherein said method comprises analysing one or more cell populations in a peripheral blood sample from the patient to determine an immune profile; and: i. stratifying the patient into a first treatment group if the immune profile determined is indicative of response to treatment with an IDO1 polypeptide, a PD-L1 polypeptide and an antibody that binds to PD1; or ii. stratifying the patient into a second treatment group if the immune profile determined in step indicates that the subject will not respond to said treatment.

Embodiment 44. The method of Embodiment 43, wherein the first treatment group is to be treated, or is treated with, the IDO1 polypeptide, the PD-L1 polypeptide and the antibody that binds to PD1 and the second treatment group is to be treated with, or is treated with, one or more alternative therapies.

Embodiment 45. The method any one of Embodiments 39-44, wherein the immune profile is a baseline immune profile.

Embodiment 46. The method of any one of Embodiments 43-45, wherein an immune profile indicative of response to treatment comprises or more of: a) a decrease in CD4+ T regulatory cells compared to a control subject population; b) a decrease in CD28+CD4+ T cells compared to a control subject population; c) an increase in LAG-3+CD4+ T cells compared to a control subject population; d) a decrease in monocytic-myeloid derived suppressor cells (mMDSCs), compared to a control subject population; e) an increase in CD56^{dim}CD16+ Natural Killer (NK) cells compared to a control subject population; f) a decrease in CD56^{bight}CD16− Natural Killer (NK) cells compared to a control subject population; and/or g) an increase in conventional dendritic cells type 2 (cDC2) compared to a control subject population.

Embodiment 46A. The method of any one of Embodiments 43-45, wherein an immune profile indicative of response to treatment comprises or more of: a) less than 6% CD4+ T regulatory cells as a percentage of CD4+ T cells; b) less than 70% CD28+CD4+ T cells as a percentage of CD4+ T cells; c) greater than 12% LAG-3+CD4+ T cells as a percentage of CD4+ T cells; d) less than 10% monocytic-myeloid derived suppressor cells (mMDSCs) as a percentage of PBMCs; e) greater than 6% CD56dimCD16+ Natural Killer (NK) cells as a percentage of PBMCs; f) less than 0.3% CD56brightCD16− NK cells as a percentage of PBMCs; and/or g) greater than 0.01% conventional dendritic cells type 2 (cDC2) as a percentage of PBMCs.

Embodiment 47. A method of monitoring the response of a cancer patient to treatment with an IDO1 polypeptide, a PD-L1 polypeptide and an antibody that binds to PD1, wherein said method comprises analysing one or more cell populations in a peripheral blood sample from the patient to determine an immune profile, and: i. determining that the patient is responding to treatment if the patient has an immune profile indicative of response to treatment; or ii. determining that the patient is not responding to treatment if the patient does not have an immune profile indicative of response to treatment.

Embodiment 48. The method of Embodiment 47, wherein the immune profile indicative of response to treatment comprises increased expression of CD28, HLA-DR, CD39, TIGIT and/or TIM-3 on CD4+ T cells and/or increased expression of HLA-DR, CD39, LAG-3 and/or TIGIT on CD8+ T-cells.

Embodiment 49. The method of any one of Embodiments 40, 43, 44 or 47, wherein the IDO1 polypeptide is as defined in Embodiment 9 or 10 and/or the PD-L1 polypeptide is as defined in any one of Embodiments 11-13 and/or the antibody that binds to PD1 is pembrolizumab or nivolumab.

Embodiment 50. The method of any one of Embodiments 31-49, wherein the cancer patient has a metastatic melanoma.

### EXAMPLES

The invention is illustrated by the following Examples.

### Example 1—Methods

### Trial Design and Treatment Plan

MM1636 is an investigator-initiated, non-randomized, open-label, single-center phase I/II study. All patients were treated at the Department of Oncology, Herlev and Gentofte Hospital, University of Copenhagen, Herlev, Denmark.

This study initially aimed to include 30 αPD1 treatment-naive patients with MM. An amendment with the addition of two other cohorts with 10 patients in each cohort was done to evaluate the immune responses and clinical efficacy in αPD1 resistant patients (cohort B de-novo resistance and cohort C acquired resistance) for a total of 50 patients. The amendment with cohort B and C was approved at inclusion of 18 patients in cohort A. The trial is still including patients in cohort B and C. This article reports results from Cohort A.

The study was conducted according to the Declaration of Helsinki and Good Clinical Practice (GCP) and monitored by the GCP-unit, Copenhagen, Denmark. The protocol was approved by the Ethical Committee of the Capital region of Denmark (H-17000988), the Danish Medical Agencies (2017011073) and the Capital Region of Denmark Data Unit (P-2019-172). The study was registered at ClinicalTrials.gov, identifier: NCT03047928 and EudraCT no: 2016-0004527-23. The first 6 patients were treated as phase 1 and evaluated for safety and tolerability before the remaining 24 patients were included in phase 2.

A schematic of the treatment plan is provided in FIG. 1B. Briefly, patients were screened after written informed content. Before beginning treatment, a baseline PET/CT scan was performed and baseline blood samples and needle biopsies were taken. Patients were treated with the IDO/PD-L1 peptide vaccine subcutaneously biweekly for the first 6 administrations and thereafter every fourth week for a maximum of 15 vaccines. Nivolumab was administered in parallel biweekly doses (3 mg/kg) up to 24 series. If patients needed subsequent nivolumab after ending the vaccination regimen, they were treated with 6 mg/kg every fourth week up to two years. Treatment with nivolumab was discontinued at the maximum benefit (investigator assessed), a maximum of 2 years therapy, at progression or due to severe adverse events. (FIG. 1B: treatment plan) Needle biopsy and delayed type hypersensitivity assays were performed after 6 series of treatment if assessable. PET/CT scans were performed every third month.

### Vaccine Composition

Each vaccine was composed of 100 μg 10102, a 21-amino-acid peptide (DTLLKALLEIASCLEKALQVF SEQ ID NO: 3) from the peptide IDO, and 100 μg 10103, a 19-amino-acid peptide (FMTYWHLLNAFTVTVPKDL SEQ ID NO: 32) from the signal peptide of PD-L1 (PolyPeptide Laboratories, France). The peptides were dissolved separately in 50 μL dimethylsulfoxide (DMSO), sterile filtered, and frozen at −20° (NUNC™ CyroTubes™ CryoLine System™ Internal Thread, Sigma-Aldrich). At <24 h before administration, the peptides were thawed. The PD-L1 peptide was diluted in 400 μL sterile water and immediately before injection mixed with the IDO peptide solution and 500 μL Montanide ISA-51 (Seppic Inc., France) to a total volume of 1 mL.

### Patients

Patients above 18 years with locally advanced or stage IV melanoma according to American Joint Committee on Cancer (AJCC) seventh edition, at least one measurable lesion according to Response Evaluation Criteria in Solid Tumours (RECIST 1.1), and an Eastern Cooperative Oncology Group (ECOG) performance status (PS) of 0-1 were eligible. Main exclusion criteria were prior treatment with αPD1 therapy, CNS metastases >1 cm, severe comorbidities, and active autoimmune disease. Enrolment was not restricted to PD-L1 status but was known prior inclusion. Patients were included after informed consent.

### Key Study Assessments

Safety and tolerability were evaluated based on changes in clinical laboratory analyses and reported adverse events. Adverse events were assessed according to Common Terminology Criteria for Adverse Events (CTCAE v. 5.0) and were graded 1-5 in all treated patients up to 6 months after the last dose of the IDO/PD-L1 vaccine.

Clinical efficacy was assessed using Fluor-18-deoxyglucose-positron emission tomography (FDG PET/CT) scans before treatment and every third month until progression. Objective response was categorized into complete response (CR), partial response (PR), stable disease (SD) or progressive disease (PD) according to RECIST v 1.1.

Blood samples for immunologic analyses were collected pre-treatment, before third cycle, after 6th, 12th, 18th, and 24th cycle (on vaccination) and 3 and 6 months after last vaccine.

Two to three tumour needle biopsies (1.2 mm) were collected at baseline and after 6 cycles from the same tumour site, when assessable, to evaluate immune responses at the tumour site.

Delayed type hypersensitivity (DTH) skin-test and punch biopsy from DTH area was performed after cycle 6 for the evaluation of skin infiltrating lymphocytes (SKILS) reactive to PD-L1 and IDO. (FIG. 1B: treatment plan).

### Clinical Outcome Statistical Analysis

Survival curves were computed by GraphPad Prism software version 8.0.2 according to Kaplan-Meier method. Median follow-up time of enrollment was calculated using the reverse Kaplan-Meier method, also in GraphPad Prism software v. 8.0.2. For binary outcomes, 95% two-sided CIs were constructed using the Clopper-Pearson method also in GraphPad.

An independent board certified and experienced onco-radiologist performed an external review to evaluate clinical response to address the potential bias of investigator site review. The external review took place at Rigshospitalet, Copenhagen University Hospital. This hospital did not participate in the MM1636 trial, and the external reviewer had no prior knowledge about the clinical trial or the trial therapy. Only PET/CT images were accessed, and arrows indicating target/non-target lesions appeared on baseline images as the only additional information.

To address potential trial bias regarding treatment effect, patients in the MM1636 trial were matched with patients from the Danish Metastatic Melanoma Database (DAMMED), a population-based database that retrospectively collects data on patients with metastatic melanoma in Denmark. 938 patients treated with αPD1 monotherapy contemporaneously (January 2015 to October 2019) were extracted. 218 of these patients were eligible for comparison and matching (all parameters available) (supplementary table 1), and 74 patients from DAMMED were found to match. Patients were matched on age (≤70, >70), gender, LDH (normal, elevated), M-stage (M1a, M1b, M1c), BRAF status (Wildtype, mutated) and PD-L1 status (<1%, ≥1%). An exact matching algorithm was used where patients in MM1636 were matched with patients from DAMMED with the same combination of variables. Twenty-nine patients from MM1636 were matched with the exact combinations of the six variables. One patient could not be matched. To secure balance of the calculations, control patients were weighted according to the number of patients for each MM1636 patient. Estimates for treatment effects were calculated by weighted logistic regression analyses and weighted Cox proportional hazard model. The R package “Matchlt” was used for matching patients.

As the method chosen for matching control patients to protocol patients, a weighted binary logistic regression model was used for comparing response rates in the two matched cohorts. Odds ratios (OR) and response rates, including their corresponding 95% confidence intervals (CI), were extracted from the regression models. All p-values were two-sided and p-values below 0.05 were considered statistically significant. SAS version 9.4M5 were used for the weighted logistic regression models.

### Processing of PBMCs

Peripheral blood was collected from all patients in heparinized tubes and was processed within 4 hours. In brief, peripheral blood mononuclear cells (PBMCs) were isolated using Lymphoprep™ (Medinor) separation. PBMCs were counted on Sysmex XP-300 and frozen in Human AB Serum (Sigma-Aldrich, Ref. No H4522-100 ml) with 10% DMSO using controlled-rate freezing (Cool-Cell, Biocision) in a −80° C. freezer and the next day moved to a −140° C. freezer until further processing.

Needle Biopsies at Baseline and after 6th Vaccine

2-3 needle biopsies (1.2 mm) were taken at baseline and after 6th cycle of treatment when assessable from the same tumour lesion. One fragment was formalin-fixed, paraffin-embedded (FFPE), one to two fragments were used for manufacturing of tumour infiltrating T-cells (TILs) and autologous tumour cell lines.

### Delayed-Type Hypersensitivity and Generation of Skin Infiltrating Lymphocytes (SKILS)

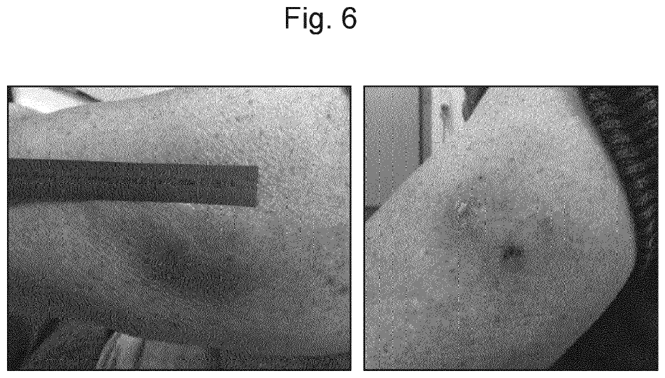

After 6 cycles of treatment, we performed intradermal injections of vaccine components without adjuvant and one control injection containing DMSO without peptide. Patients were injected with either a mix of both IDO and PD-L1 at all three injection sites or PD-L1, IDO or a mix at the injection's sites respectively. (supplementary FIG. 6) 8 hours after injection punch 527 biopsies were resected from the three sites where the peptide was injected and transported immediately to the laboratory and cut into 1 to 2 mm³ fragments.

Skin infiltrating lymphocytes (SKILS) were expanded to establish “young SKILS” in CM media consisting of RPMI1640 with GlutaMAX, 25 mMHEPES pH 7.2 (Gibco, 72400-021), Interleukin 2 (100/6000 IU/mL) (IL-2; Proleukin Novartis, 004184), 10% heat inactivated hum AB serum (HS; Sigma-Aldrich, H4522-100 ML), 100 U/mL penicillin, 1.25 μg/mL Fungizone (Bristol-Myers Squibb, 49182) 100 μg/ml streptomycin (Gibco, 15140-122). Half of the medium was replaced three times per week.

Young SKILS from IDO, PD-L1 and mix were further expanded in a small-scale version of the 14-day rapid expansion protocol, as previously described (Donia, M. et al. Characterization and comparison of ‘Standard’ and ‘Young’ tumor infiltrating lymphocytes for adoptive cell therapy at a Danish Translational Research Institution. Scand. J. Immunol. 157-167 (2011)).

### Quantification of Vaccine-Specific T-Cells in Blood by ELISPOT

For enumeration of vaccine-specific T-cells in the peripheral blood, PBMCs from patients were stimulated with IDO or PD-L1 peptide in the presence of low dose IL-2 (120U/mL) for 7 to 13 days before being used in IFN-γ ELISPOT.

Briefly, cells were placed in a 96-well PVDF membrane bottomed ELISPOT plate (MultiScreen MSIPN4W50; Millipore) pre-coated with IFN-γ capture Ab (Mabtech). Diluted IDO or PDL1 peptide stock in DMSO was added at 5 M, an equivalent amount of DMSO was added to control wells. PBMCs from each patient were set up in duplicates or triplicates for peptide and control stimulations. Cells were incubated in ELISPOT plates in the presence of the peptide for 16-18 hours after which they were washed off and biotinylated secondary Ab (Mabtech) was added. After 2 h incubation unbound secondary antibody was washed off and streptavidin conjugated alkaline phosphatase (AP) (Mabtech) was added for 1 h. Next, unbound streptavidin conjugated enzyme was washed off and the assay was developed by adding BCIP/NBT substrate (Mabtech). ELISPOT plates were analyzed on CTL ImmunoSpot S6 Ultimate-V analyzer using Immunospot software v5.1. Responses were calculated as the difference between average numbers of spots in wells stimulated with IDO or PDL1 peptide and corresponding control wells.

Statistical analysis of ELISPOT responses was performed using DFR method as described by Moodie et al. using RStudio software (RStudio Team, 2016; RStudio: Integrated Development for R. RStudio, Inc., Boston, MA, available at the rstudio website) (Moodie, Z. et al. Cancer Immunol. Immunother. 59, 1489-1501 (2010)).

Vaccine specific ELISPOT response was defined as true if the difference between the spot count in control and peptide stimulated wells was statistically significant according to the DFR statistical analysis or for samples performed in duplicates if the spot count in peptide stimulated wells was at least 2× the spot count in the control wells (Moodie, Z. et al. Cancer Immunol. Immunother. 59, 1489-1501 (2010)).

Quantification of Vaccine-Specific T-Cells from DTH Biopsy Site by ELISPOT

In vitro expanded SKILs were rested in media without IL-2 overnight before being used in IFNγ ELISPOT as described above to evaluate reactivity of skin infiltrating T-cells.

Generation of IDO and PD-L1 Specific T-Cell Cultures from PBMCs or SKILs

IDO or PD-L1 specific T-cells were isolated from peptide stimulated in vitro PBMC cultures on day 14-15 after stimulation or in vitro expanded SKILs cultures. For specific T-cell isolation, PBMCs or SKILs were stimulated with IDO or PD-L1 peptide and cytokine-producing T-cells were sorted using IFN-γ or TNF-α Secretion Assay—Cell Enrichment and Detection Kit (Miltenyi Biotec).

### Cytokine Production Profile of PD-L1 and IDO Specific T-Cells by Intracellular Staining

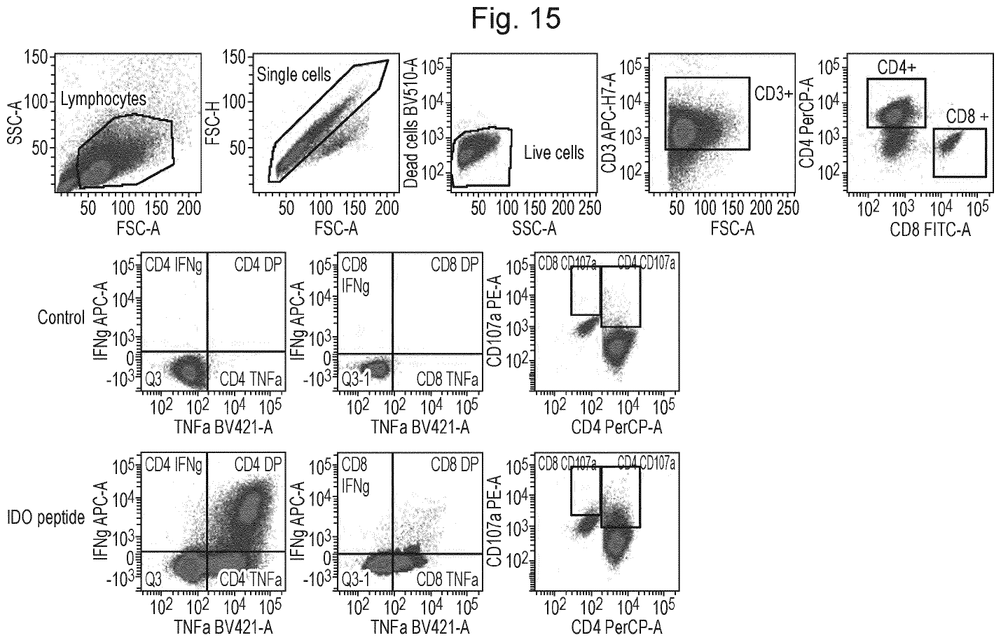

To assess the T-cell cytokine production profile, isolated and expanded IDO and PD-L1 specific Tcell cultures were stimulated for 5 hours with 5 M of peptide in a 96-well plate. 1 hour after the start of the incubation, CD107α-PE (BD Biosciences cat. 555801) antibody and BD GolgiPlug™ (BD Biosciences) was added at a dilution of 1:1000. After the 5-hour incubation the cells were stained using fluorescently labelled surface marker antibodies: CD4+-PerCP (cat. 345770), CD8+− FITC (cat. 345772), CD3-APC-H7 (cat. 560275) (all from BD Biosciences). Dead cells were stained using FVS510 (564406, BD Biosciences), followed by an overnight fixation and permeabilization using eBioscience™ Fixation/Permeabilization buffers (eBioscience, cat. 00-5123-43, 00-5223-56) according to the manufacturer's instructions. Cells were then stained intracellularly in eBioscience permeabilization buffer (eBioscience, cat. 00-8333-56) with IFNg-APC (cat. 341117), TNFa-BV421 (cat. 562783). Samples were analyzed on FACSCanto™ II (BD Biosciences) using BD FACSDiva software version 8.0.2. Gating strategy is shown in FIG. 15.

### BRAF Mutational Status and PD-L1 Status at Baseline on all Patients Evaluated Locally at Herlev and Gentofte Hospital, Denmark on Historical FFPE Biopsies

A library of historical formalin-fixed, paraffin-embedded (FFPE) biopsies were assessable on all patients and analysed locally at Herlev and Gentofte University Hospital by experienced pathologists for BRAF status and PD-L1 expression on tumour cells.

The BRAF analysis was carried out with Real-Time PCR with the EntroGen BRAF Mutation Analysis Kit II (BRAFX-RT64, CE-IVD) to specifically detect V600D, V600E and V600K mutations in the BRAF gene.

PD-L1 status was assessed using the monoclonal Rabbit Anti-PD-L1, clone 28.8 (PD-L1 IHC 28-8 pharmDx) in FFPE. Patients were considered PD-L1 positive with expression levels ≥1% and negative with expression levels <1%.

### HLA-Type

Blood samples from all 30 patients were genotyped for Class I (HLA-A, HLA-B, HLA-C) and three class II (HLA-DRB1, HLA-DQA1, HLA-DQB1) using LinkSēq™ HLA Typing Kits (thermofischer (1580 C)). These test kits are based on real-time polymerase chain reaction (PCR) using allele-specific exponential amplification (sequence-specific primers) followed by melting curve analyses.

### Immunohistochemistry (IHC) Simplex: Immunoscore® CR: T Lymphocytes (TL), MHCI, MHCII, IDO, PD-L1

IHC staining was performed at HalioDx Service Laboratory using a qualified Ventana Benchmark XT with 4 different steps: 1) Antigen retrieval; 2) Staining with primary antibody (CD3, CD8+, MHCI, MHCII, IDO and PD-L1); 3) Detection with a secondary antibody using ultraView Universal DAB Detection Kit. 4) Counterstaining using Hematoxylin & Bluing Reagent (Staining of cellular nuclei).

Control slides were systematically included in each staining run to permit quality control of the obtained measurements. Following coverslipping, slides were scanned with the NanoZoomer-XR to generate digital images (20×). Two consecutive slides were specifically used to perform Immunoscore® CR TL CD3+ cells and CD8+ cells staining.

### Digital Pathology of T Lymphocytes (TL)

The digital pathology for Immunoscore® CR TL allowed the quantification of positive cells (in cells/mm²) into the core tumour and invasive margin if present. Each sample was analyzed using HalioDx Digital Pathology Platform.

### Digital Pathology IDO and Pathologist Analysis of IDO

The digital pathology for IDO allowed the quantification of stained area (in mm2) into the whole tumour. Each sample was analyzed using HalioDx Digital Pathology Platform. Analysis of IDO+ cells was performed by a pathologist. Results were expressed as an H-score from 0 to 300. The score is obtained by the formula: 3× percentage of cells with a strong staining+2× percentage of cells with a moderate staining+percentage of cells with a weak staining.

### Digital Pathologist Analysis of MHCI, MHCII

Analysis of MHCI and MHCII+ cells was performed by a pathologist. Results were expressed as a percentage of positive cells of tumour cells. Digital Pathology Immunoscore® Immune checkpoint (CD8+/PD-L1) Immunoscore® CR IC test allowed the quantification of CD8+ cell density into the whole tumour and CD8+-centered proximity index (which corresponds to the percentage of CD8+ cells that have at least a PD-L1+ cell in the neighbourhood) at different cut-off distance (20 m, 40 m, 60 m and 80 m).

### Pathologist Analysis of PD-L1

A pathologist performed analysis of PD-L1+ cells. Positivity on a viable tumour cell of cells was considered when partial or complete cell membrane staining was observed (more than 10% of the tumour cell membrane). Results were expressed as a percentage.

### Nanostring RNA Profiling and Immunosign®

RNA was extracted from Formalin fixed paraffin embedded (FFPE) tissues using QIAGEN RNeasy FFPE extraction kits (QIAGEN GmbH, Hilden, Germany). Annotations from the pathologist performing H&E staining were used to guide removal of normal tissue from the slides by macrodissection prior to nucleic acid extraction, which occurred after tissue deparaffinization and lysis. Each extracted RNA was independently quantified using a NanoDrop spectophotometer (NanoDrop Technologies, Oxfordshire, UK) and qualified (Agilent Bioanalyzer, Santa Clara, United States). Degradation assessment was quantified as the percentage of RNA fragments smaller than 300 base pairs using RNA 6000 Nano Kit (Agilent Bioanalyzer, Santa Clara, United States). Good sample quality was defined as less than 50% of RNA fragments of 50 to 300 base pairs in size.

RNA expression profiling was performed using the nCounter®PanCancer Immune Profiling Panel from NanoString (NanoString Technologies, Seattle, USA). The PanCancer Immune Profiling Panel contains 776 probes and was supplemented with 6 genes to complete HalioDx Immunosign® targets.

Hybridization was performed according to manufacturer's instructions. Hybridized probes were then purified and immobilized on a streptavidin-coated cartridge using the nCounter Prep Station (NanoString Technologies). Data collection was carried out on the nCounter Digital Analyzer (NanoString Technologies) following the manufacturer's instructions to count individual fluorescent barcodes and quantify target RNA molecules present in each sample. For each assay, a scan of 490 fields of view was performed.

Raw data from the NanoString nCounter were processed using NanoString® recommendation. The quality control enables to keep good quality data with a binding density that range between 0.05 to 2.25. The linearity of positive controls was check using the R2 of regression between the counts and the concentration of positive controls. Samples that show an R2<0.75 were flagged and removed from the analysis. The background was removed using thresholding method 661 at the mean+2 standard deviation of negative controls. The raw counts were normalized using positive normalization factor.

Samples showing positive normalization factors out of the range 0.3 to 3 were removed from the analysis. A second normalization was performed using Housekeeping gene normalization factor. Only the most stable housekeeping genes were selected for this normalization step using the Variance vs Mean relationship. All samples showing a normalization factor out of the range 0.1 to 10 were removed from the analysis. All statistical analyses were performed on the normalized counts using R software (Version 2.6.2, 2019-12-12)

### T-Cell Receptor Variable Beta Chain Sequencing

To track longitudinal immune responses to therapy genomic DNAs (gDNA) was extracted from longitudinal pre- and post-treatment peripheral blood mononuclear cells (5 patients), pre- and post-treatment biopsies (5 patients) (both FFPE and RNA-later) and IDO and PD-L1 specific T-cell cultures from PBMCs (5 patients) or SKILs (1 patient).

DNA from PBMCs or RNA-later biopsies were extracted with Dneasy Blood and Tissie KIT (Qiagen, 69504), the DNA from sorted IDO and PD-L1 cells from either PBMCs or SKILs were extracted using QIAamp DNA micro Kit (Qiagen, 565304) and DNA from PFFE biopsies were extracted using Maxwell RSC DNA FFPE Kit (Promega, AS1450).

Immunosequencing of the CDR3 regions of human TCRP chains was performed using the immunoSEQ Assay (Adaptive Biotechnologies, Seattle, WA). Extracted genomic DNA was amplified in a bias-controlled multiplex PCR, followed by high-throughput sequencing. Sequences were collapsed and filtered in order to identify and quantitate the absolute abundance of each unique TCRP CDR3 region for further analysis as previously described (Robins, H. S. et al. Blood 114, 4099-4107 (2009); Carlson, C. S. et al. Nat. Commun. 4, 1-9 (2013); Robins, H. et al. J. Immunol. Methods 375, 14-816 19 (2012)).

### Statistical Analyses of TCR-β Sequencing Results

Two quantitative components of diversity were compared across samples in this study. First, Simpson Clonality was calculated on productive rearrangements by: √Σpi 2 R i=1, where R is the total number of rearrangements and pi is the productive frequency of rearrangement i. Values of Simpson Clonality range from 0 to 1 and measure how evenly receptor sequences (rearrangements) are distributed amongst a set of T-cells. Clonality values approaching 0 indicate a very even distribution of frequencies, whereas values approaching 1 indicate an increasingly asymmetric distribution in which a few clones are present at high frequencies.

Second, sample richness was calculated as the number of unique productive rearrangements in a sample after computationally downsampling to a common number of T-cells to control for variation in sample depth or T-cell fraction. Repertoires were randomly sampled without replacement five times and report the mean number of unique rearrangements.

T-cell fraction was calculated by taking the total number of T-cell templates and dividing by the total number of nucleated cells. Total number of nucleated cells were derived from reference genes using the immunoSEQ Assay.

To identify enriched vaccine clones in each patient, rearrangement frequencies in their baseline PBMC and each IDO/PD-L1 sorted T-cell sample were compared using a binomial distribution framework as previously described (DeWitt, W. S. et al. J. Virol. 89, 4517-4526 (2015)). In brief, for each clone we performed a 2-sided test that frequencies were the same in the patient's periphery and an PD-L1 or IDO T-cell sample. The Benjamini-Hochberg procedure was used to control false discovery rate (FDR) at 0.01 (Benjamini, Y. & Gavrilov, Y. Ann. Appl. Stat. 3, 179-198 (2009)). Clonal expansion in post-treatment samples were similarly assessed using this differential abundance framework but replacing an IDO/PD-L1 T-cell sample with a post-treatment series sample. In biopsies, the 6 series frequencies were compared to baseline tissue. Lastly, vaccine associated clones were tracked in each PBMC and tissue sample by summing the frequency of each rearrangement enriched in either PD-L1 or IDO T-cells. All statistical analyses were performed in R version 3.6.1.

### Cancer Cell Lines and Tumour Conditioned Media

Autologous melanoma cell lines were established from needle biopsies. Briefly, biopsies were chopped into small fragments and seeded in 24-well culture in RPMI1640 with GlutaMAX, 25 mM HEPES pH 7.2 (Gibco, 72400-021), 10% heat inactivated fetal bovine serum (Life Technologies, 10500064), 100 U/mL penicillin, 1.25 μg/mL Fungizone (Bristol-Myers Squibb, 49182) 100 μg/mL streptomycin (Gibco, 15140-122). Established adherent melanoma tumour cell lines were cryopreserved at −140° C. in freezing media containing foetal bovine serum with 10% DMSO. PD-L1 and HLA II expression on established tumour cell lines were assessed by flow cytometry staining with PD-L1− PE-Cy7 (cat. 558017) and HLA II-FITC (cat. 555558) antibodies.

To obtain tumour conditioned media (TCM), established tumour cell lines were cultured in 175 cm² Nunc cell culture flasks until 80-90% confluency was reached. The culture media was then replaced with 20 mL of fresh X-VIVO 15 with Gentamycin and Phenol Red (Lonza, BE02-060Q), media with 5% heat inactivated hum AB serum (HS; Sigma-Aldric, H4522-100 ML). After 24 h incubation, the TCM was collected and centrifuged to remove any resuspended cells, after which TCM was aliquoted, frozen and stored at −80° C.

Acute monocytic leukaemia cell line MonoMac1 was obtained from DSMZ (ACC 252) and cultured in RPMI1640 with GlutaMAX, 25 mM HEPES pH 7.2 (Gibco, 72400-021) with 10% heat inactivated foetal bovine serum.

### Isolation of Autologous Myeloid Cells

Autologous CD14+ cells were sorted from freshly thawed PBMCs using magnetic bead separation kit (Miltenyi Biotec, 130-050-201) according to the manufacturer's instruction. Isolated CD14+ cells were used as targets in IFNγ ELISPOT directly after sorting or were differentiated in vitro into tumour-associated macrophages by culturing with 1 mL fresh X-VIVO 15 media with Gentamycin and Phenol Red (Lonza, BE02-060Q), and 5% heat inactivated human AB serum, supplemented with 1 mL of autologous TCM in 24-well plate for 2 days.

### Cytokine Production Profile of PD-L1 and IDO Specific T Cells by Intracellular Staining

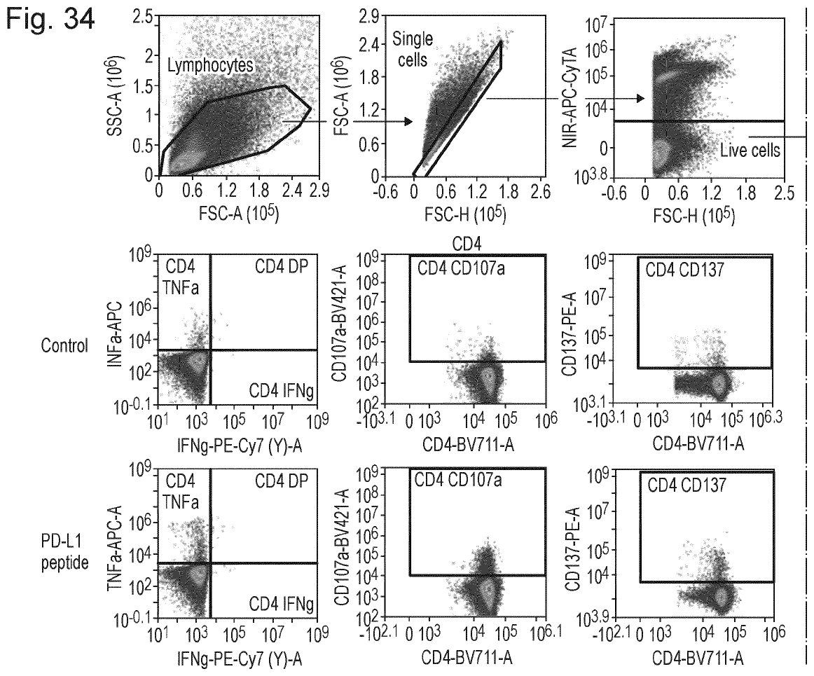

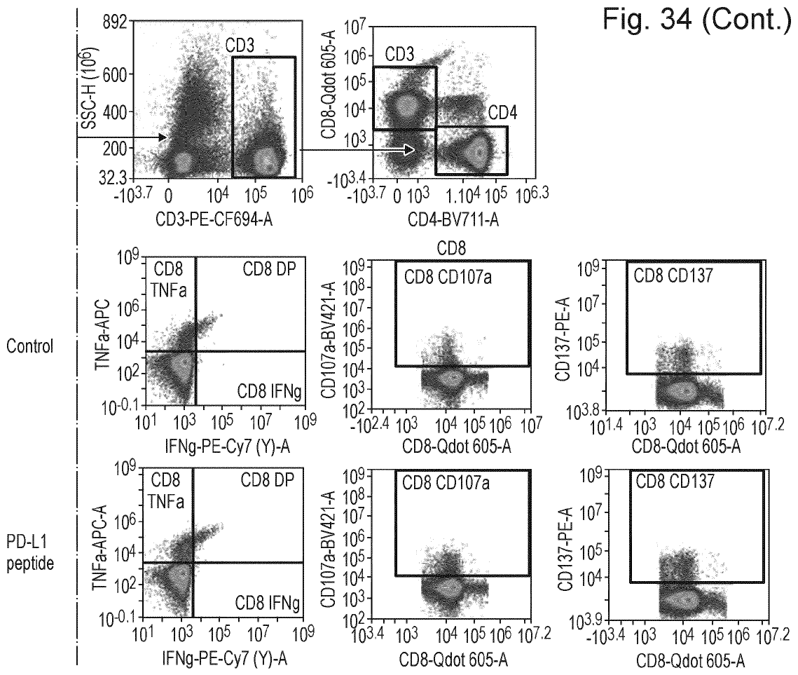

To assess the T cell cytokine production profile, isolated and expanded IDO and PD-L1 specific T cell cultures were stimulated for 5 hours with 5 μM of peptide in a 96-well plate. After 1 hour of the start of the incubation, CD107α-PE (BD Biosciences cat. 555801) antibody and BD GolgiPlug (BD Biosciences) was added at a dilution of 1:1000. After the 5-hour incubation the cells were stained using fluorescently labelled surface marker antibodies: CD4+-PerCP (cat. 345770), CD8+− FITC (cat. 345772), CD3-APC-H7 (cat. 560275) (all from BD Biosciences). Dead cells were stained using FVS510 (564406, BD Biosciences), followed by an overnight fixation and permeabilization using eBioscience™ Fixation/Permeabilization buffers (eBioscience, cat. 00-5123-43, 00-5223-56) according to the manufacturer's instructions. Cells were then stained intracellularly in eBioscience permeabilization buffer (eBioscience, cat. 00-8333-56) with IFNγ-APC (cat. 341117), TNFα-BV421 (cat. 562783). Samples were analyzed on FACSCanto II (BD Biosciences) using BD FACSDiva software version 8.0.2. To assess ex vivo T-cell reactivity to IDO and PD-L1 peptides in patient PBMCs, cells were thawed and rested for 1-2 days in media containing DNase I (1 μg/mL, SigmaAldrich, cat 11284932001). PBMCs were then stimulated with 5 μM of peptide in a 96-well plate for 8 hours. An hour after the addition of peptide CD107α-BV421 (cat. 328626) antibody and BD GolgiPlug™ (BD Biosciences) were added at a dilution of 1:1000. Surface and intracellular staining were performed as described above. Antibodies used for surface staining: CD3-PE-CF594 (cat. PE701 CF594), CD4-BV711 (cat. 563028), CD8-Qdot605 (cat. Q10009). Antibodies used for intracellular staining: CD137-PE (cat. 555956), IFNγ-PE-Cy7 (cat. 557643), TNFα-APC (cat. 554514). Samples acquired on NovoCyte Quanteon (ACEA Biosciences) and analysed using NovoExpress software vl. 4.1. To asses vaccine-specific T cell responses, background values seen in non-stimulated PBMC samples were subtracted from values seen in peptide stimulated conditions. Positive response values were set at 0.2% difference from the background values. Based on this response cut-off only TNFα, CD107α, and CD137 responses were detected in this assay. Statistical analysis comparing baseline with on/post treatment cytokine profiles were performed by using Wilcoxon matched pairs signed rank test. Gating strategy is shown in FIG. 34.

siRNA-Mediated PD-L1 and IDO Silencing

Stealth siRNA duplex for targeted silencing of PD-L1 (Invitrogen) (Hobo, W. et al. Blood 116, 4501-4511 1006 (2010)), custom silencer select siRNA for targeted silencing of IDO (Ambion), and recommended Silencer select Negative control (Ambion) siRNA for mock transfection were used.

The Stealth PD-L1 siRNA duplex consisted of the sense sequence 5′-CCUACUGGCAUUUGCUGAACGCAUU-3′ (SEQ ID NO: 33) and the antisense sequence 5′-AAUGCGUUCAGCAAAUGCCAGUAGG-3′ (SEQ ID NO: 34). Three silencer IDO siRNA duplexes were used were: siRNA1 (sense sequence 5′-ACAUCUGCCUGAUCUCAUAtt-3′ (SEQ ID NO: 35), antisense: 5′-UAUGAGAUCAGGCAGAUGUtt-3′ (SEQ ID NO: 36)); siRNA2 (sense sequence 5′-CCACGAUCAUGUGAACCCAtt-3′ (SEQ ID NO: 37), antisense: 5′-UGGGUUCACAUGAUCGUGGat-3′ (SEQ ID NO: 38)); siRNA3 (sense sequence 5′-CGAUCAUGUGAACCCAAAAtt-3′ (SEQ ID NO: 39), antisense 5′-UUUUGGGUUCACAUGAUCGtg-3′ (SEQ ID NO: 40)). For PD-L1 or IDO silencing experiments cancer cells were electroporated with 0.025 nmol of each siRNA duplex as previously described (Met, Ô., Balslev, E., Flyger, H. & Svane, I. M. Breast Cancer Res. Treat. 1009 125, 395-406 (2011)). For PD-L1 silencing experiments cancer cells were treated with IFN-γ (500 U/mL, PeproTech) 1 hour after electroporation. Electroporated cells were used as target cells in ELISPOT and ICS assays 24 h or 48 h after siRNA electroporation.

### RT-qPCR

Total RNA was extracted using the RNEasy Plus Mini Kit (Qiagen, cat. 74134) following manufacturer's instructions. RNA concentration was quantified using a NanoDrop 2000 (Thermo Fisher Scientific) and a total of 1000 ng RNA was reverse transcribed using the High Capacity cDNA Reverse Transcription Kit (Applied Biosystems, cat. 4368814) using 1000 ng input RNA for transcription. Real time qPCR analyses were performed using TaqMan Gene Expression Assay on a Roche Lightcycler 480 Instrument. RT-qPCRs were performed in a minimum of 3 technical replicates and analyzed using the dCt-method as described in Bookout et al. (Curr. Protoc. Mol. Biol. 73, 1012 1-28 (2006)) with normalization of IDO1 expression (primerID: Hs00984148_ml) or PD-L1 expression (primerID: Hs001125296_ml) to the expression level of the housekeeping gene POL2RA (primerID: Hs00172187_ml) and control sample (mock). For low concentration samples with no amplification, Ct was set to 40. No-reverse transcriptase controls were used as controls for specific amplifications. P values determined using two-tailed parametric t test.

### Example 2—Clinical Trial Results

In this MM1636 phase I/II clinical trial, patients with metastatic melanoma received a combination of the IDO/PD-L1 (I0102/I0103) peptide vaccine with the adjuvant montanide and the αPD-1 antibody nivolumab. Patients were included in 3 cohorts: 30 αPD1 therapy naive (Cohort A), 10 αPD1 therapy refractory (cohort B, de-novo resistance) and 10 patients who progressed after αPD1 therapy (cohort C, acquired resistance). Results are provided herein for Cohort A.

### Treatment Protocol and Patients

The treatment protocol is described above in Example 1 and exemplified in FIG. 1B. 30 patients were enrolled from December 2017 to June 2020. None of the 30 patients dropped out of the study; all received at least 3 cycles of therapy. At the current database lock (Oct. 5, 2020) six patients were still on treatment within the trial. Of the 24 patients who are not on trial treatment at data cut-off, two are still receiving nivolumab monotherapy (6 mg/kg q4w). Reasons for stopping treatment for the remaining 22 patients were due to disease progression (37%), toxicity (20%), maximum benefit/CR confirmed on two consecutive scans (17%) or completed two years of treatment (7%).

For the 24 patients who were not on trial treatment at data cut-off, the mean number of vaccinations was 10.5 (range 3-15). Thirteen of these 24 patients continued nivolumab 6 mg/kg, q4w, as a standard of care. Nine patients received subsequent therapy after progression (Table 2). Baseline characteristics are shown in Table 1. Mean age was 70 years, 38% had elevated LDH, 60% were M1c, 38% were BRAF mutated, 43% were PD-L1 negative (<1%). A total of three patients (10%) had received prior ipilimumab therapy. (Table 3).

TABLE 1
Baseline Patient Characteristics (n = 30)

| Characteristic | % patients | |
|---|---|---|
| Mean age, years (range) | 70 | (46-85) |
| Sex - Male no (%) | 16 | (55%) |
| ECOG-PS 0 no. (%) | 26 | (87%) |
| Lactate Dehydrogenase (LDH) levels | | |
| ≤ULN | 19 | (63%) |
| >ULN | 11 | (37%) |
| M stage - no. (%) AJCC-8 | | |
| M1a | 6 | (20%) |
| M1b | 6 | (20%) |
| M1c | 18 | (60%) |
| Number of lesion sites | | |
| 1 | 6 | (20%) |
| 2-3 | 23 | (57%) |
| >3 | 7 | (23%) |
| Liver metastases yes no. (%) | 10 | (33%) |
| BRAF-status no. (%) | | |
| Mutant | 11 | (37%) |
| Wild-type | 19 | (63%) |
| PD-L1 no. (%) | | |
| <1% | 13 | (43%) |
| >1% | 17 | (57%) |
| Previous systemic therapy | | |
| Ipilimumab | 3 | (10%) |
| No | 27 | (90%) |

TABLE 2
Doses of vaccines and nivolumab administered and reason to stop treatment plus subsequent therapy (n = 30)

| | | | Number | Number | | |
|---|---|---|---|---|---|---|
| | | Number | nivolumab | nivolumab | | |
| | | vaccines | series | series | | |
| Patient ID | BOR | series | (3 mg/kg) | (6 mg/kg) | Reason stop treatment | Subsequent therapy |
| MM1636.01 | CR | 15 | 24 | 5 | Grade 3 rash | |
| MM1636.02 | PD | 5 | 5 | | Progression | Dabrafenib/Trametinib |
| MM1636.03 | CR | 15 | 24 | 9 | Maximum benefit | |
| MM1636.04 | PD | 5 | 5 | | Progression | Radiotherapy, Temodal |
| MM1636.05 | CR | 13 | 21 | | Grade 2 pneumonitis | |
| MM1636.06 | CR | 15 | 24 | 9 | Progression | Dabrafenib/Trametinib |
| MM1636.07 | CR | 8 | 12 | 2 | Grade 2 pneumonitis | Reinduction Nivolumab |
| MM1636.08 | PR | 11 | 16 | | Progression | Ipilimumab, Radiotherapy |
| MM1636.09 | PD | 5 | 5 | | Progression | |
| MM1636.10 | PR | 15 | 24 | 7 | Progression | Encorafenib/Binimetinib |
| MM1636.11 | PD | 3 | 3 | | Progression | Dabrafenib/Trametinib, |
| | | | | | | Ipilimumab |
| MM1636.12 | CR | 15 | 24 | 8 | Maximum benefit | |
| MM1636.13 | PR | 15 | 24 | 13 | Reached 2 years of treatment | |
| MM1636.14 | CR | 11 | 16 | | Grade 2 colitis | |
| MM1636.15 | PR | 15 | 24 | 11 | Reached 2 years of treatment | |
| MM1636.16 | PR | 9 | 12 | | Progression | T cell therapy CT ID: |
| | | | | | | NCT02278887 |
| MM1636.17 | PD | 6 | 6 | | Progression | |
| MM1636.18 | CR | 5 | 5 | | Grade 5 toxicity due to | |
| | | | | | nivolumab | |
| MM1636.20 | CR | 11 | 18 | 2 | Maximum benefit | |
| MM1636.22 | CR | 15 | 24 | 1 | Grade 3 atralgia | |
| MM1636.23 | CR | 15 | 24 | 3 | Maximum benefit | |
| MM1636.24 | CR | 15 | 24 | | Maximum benefit | |
| MM1636.27 | PR | 15 | 24 | 2 | Treatment ongoing | |
| MM1636.29 | PR | 15 | 24 | 2 | Treatment ongoing | |
| MM1636.34 | PD | 3 | 3 | | Progression | |
| MM1636.35 | PR | 21 | 13 | | Treatment ongoing | |
| MM1636.37 | PR | 7 | 8 | | Progression | Encorafenib/Binimetinib |
| MM1636.38 | CR | 18 | 12 | | Treatment ongoing | |
| MM1636.39 | PR | 16 | 11 | | Treatment ongoing | |
| MM1636.42 | PR | 7 | 6 | | Treatment ongoing | |

TABLE 3
Patient Characteristics

| | | | | | | | | PD-L1 | | |
|---|---|---|---|---|---|---|---|---|---|---|
| | | | | | Sum of target | M-stage | M-stage | status | | LDH at |
| | | | Previous | Metastatic sites | lesions at | AJCC 7^{th} | AJCC 8^{th} | (clone | BRAF | baseline |
| Patient ID | Gender | Age | treatment | at baseline | baseline (mm) | edition | edition | 28.8) | status | (U/L) |
| MM1636.01 | M | 85 | None | LN, SC, adrenal | 33 | M1c | M1c(1) | <1% | Wildtype | 279 |
| | | | | glands, peritoneal | | | | | | |
| | | | | carcinomatosis, bone | | | | | | |
| MM1636.02 | F | 69 | None | LN, tumor along | 216 | M1c | M1c(1) | <1% | Mutated | 1510 |
| | | | | iliaca artery, SC | | | | | | |
| MM1636.03 | F | 79 | None | Pericardial fat, Lung | 60 | M1c | M1c(1) | >1% | Mutated | 260 |
| MM1636.04 | F | 77 | 1. line | LN, Lung | 62 | M1b | M1b(0) | <1% | Wildtype | 183 |
| | | | Ipilimumab | | | | | | | |
| MM1636.05 | M | 72 | None | LN, peritoneal | 88 | M1c | M1c(1) | >1% | Wildtype | 324 |
| | | | | carcinomatosis, | | | | | | |
| | | | | pleural | | | | | | |
| | | | | carcinomatosis, SC | | | | | | |
| MM1636.06 | F | 67 | Adjuvant | LN | 19 | M1a | M1a(0) | >1% | Mutated | 180 |
| | | | Ipilimumab | | | | | | | |
| MM1636.07 | M | 72 | None | LN | 43 | M1a | M1a(0) | >1% | Wildtype | 134 |
| MM1636.08 | F | 76 | 1. line | LN, SC, tumor near | 191 | M1c | M1c(1) | <1% | Wildtype | 269 |
| | | | Ipilimumab | liver, tumor in pelvis, | | | | | | |
| | | | | bone marrow tibia | | | | | | |
| MM1636.09 | F | 72 | none | Lung, liver, | 72 | M1c | M1c(0) | <1% | Wildtype | 255 |
| | | | | adrenal gland | | | | | | |
| MM1636.10 | M | 46 | none | Lung, pleura, | 11 | M1c | M1c(0) | >1% | Mutated | 132 |
| | | | | adrenal gland | | | | | | |
| MM1636.11 | F | 67 | none | LN, SC | 21 | M1a | M1a(0) | <1% | Mutated | 187 |
| MM1636.12 | F | 77 | none | LN | 26 | M1a | M1a(0) | >1% | Mutated | 251 |
| MM1636.13 | F | 59 | none | LN, SC, Lung, | 77 | M1c | M1c(1) | >1% | Wildtype | 252 |
| | | | | liver, bone | | | | | | |
| MM1636.14 | M | 54 | none | LN, SC, gall | 76 | M1c | M1c(0) | <1% | Mutated | 192 |
| | | | | bladder, lung | | | | | | |
| MM1636.15 | M | 80 | none | LN, liver, rectal | 26 | M1c | M1c(1) | <1% | Wildtype | 309 |
| MM1636.16 | M | 46 | none | Lung, liver | 14 | M1c | M1c(0) | >1% | Mutated | 203 |
| MM1636.17 | M | 77 | none | Lung, SC | 12 | M1b | M1b(0) | >1% | Wildtype | 190 |
| MM1636.18 | F | 80 | none | Lung, SC | 23 | M1b | M1b(0) | <1% | Wildtype | 164 |
| MM1636.20 | M | 56 | none | LN, liver | 44 | M1c | M1c(0) | <1% | Mutated | 145 |
| MM1636.22 | M | 76 | none | LN, Lung | 73 | M1b | M1b(0) | >1% | Wildtype | 178 |
| MM1636.23 | F | 76 | none | LN, pleura, spleen | 47 | M1c | M1c(0) | <1% | Wildtype | 173 |
| MM1636.24 | F | 67 | none | adrenal gland | 31 | M1c | M1c(0) | >1% | Wildtype | 168 |
| MM1636.27 | M | 82 | none | SN, intramuscular, | 55 | M1c | M1c(0) | >1% | Mutated | 236 |
| | | | | liver, LN | | | | | | |
| MM1636.29 | M | 74 | none | LN, Lung, SC | 13 | M1b | M1b(0) | >1% | Wildtype | 165 |
| MM1636.34 | M | 75 | none | LN, Lung, liver | 41 | M1c | M1c(1) | <1% | Wildtype | 314 |
| MM1636.35 | M | 52 | none | lung, liver, SC | 15 | M1c | M1c(1) | >1% | Wildtype | 206 |
| MM1636.37 | F | 76 | none | LN | 42 | M1a | M1a(0) | <1% | Mutated | 232 |
| MM1636.38 | F | 53 | none | SC | 25 | M1a | M1a(1) | >1% | Wildtype | 270 |
| MM1636.39 | M | 80 | none | lung | 34 | M1b | M1b(0) | >1% | Wildtype | 180 |
| MM1636.42 | M | 81 | none | liver, SC | 144 | M1c | M1c(1) | >1% | Wildtype | 317 |

### Example 3—Clinical Results

### Combination of the IDO/PD-L1 Vaccine and Nivolumab Leads to Notable Clinical Responses

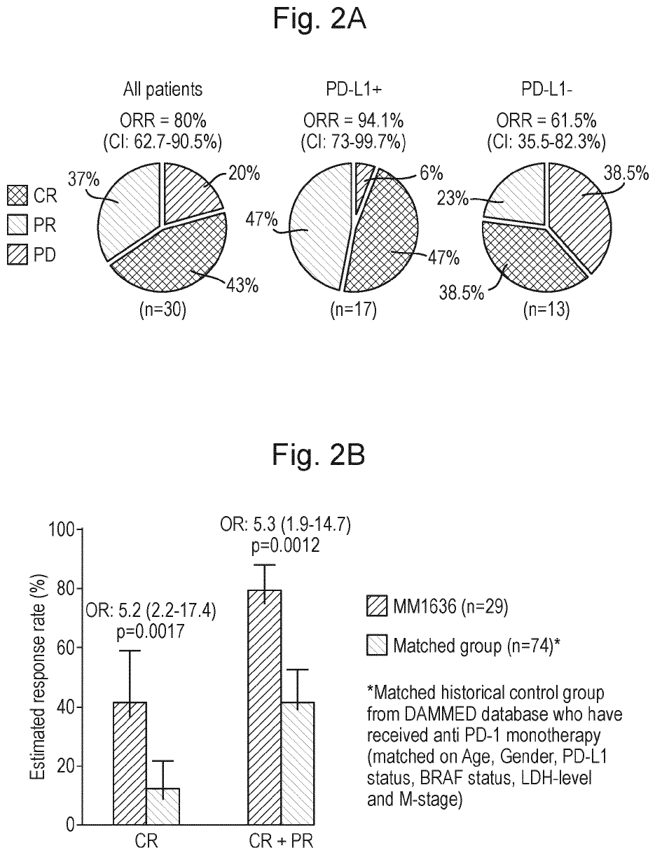

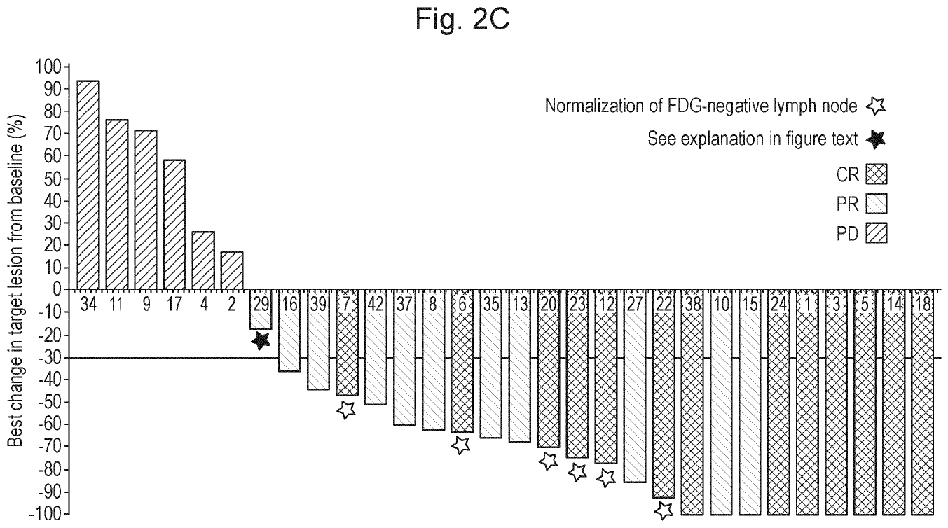

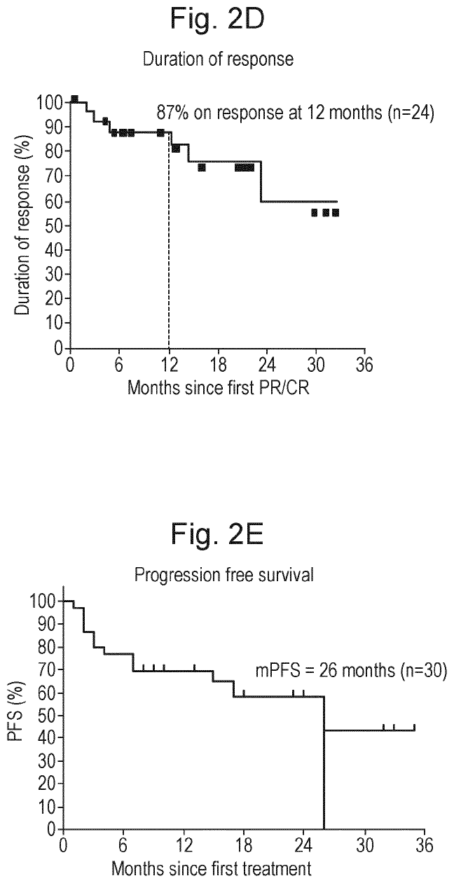

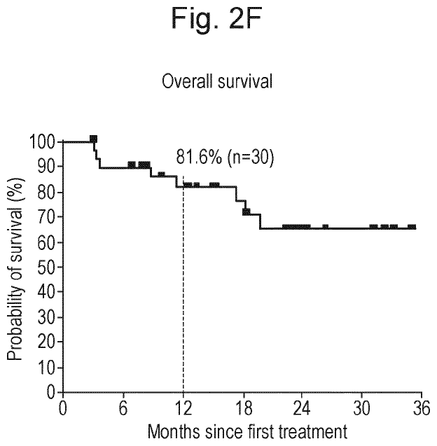

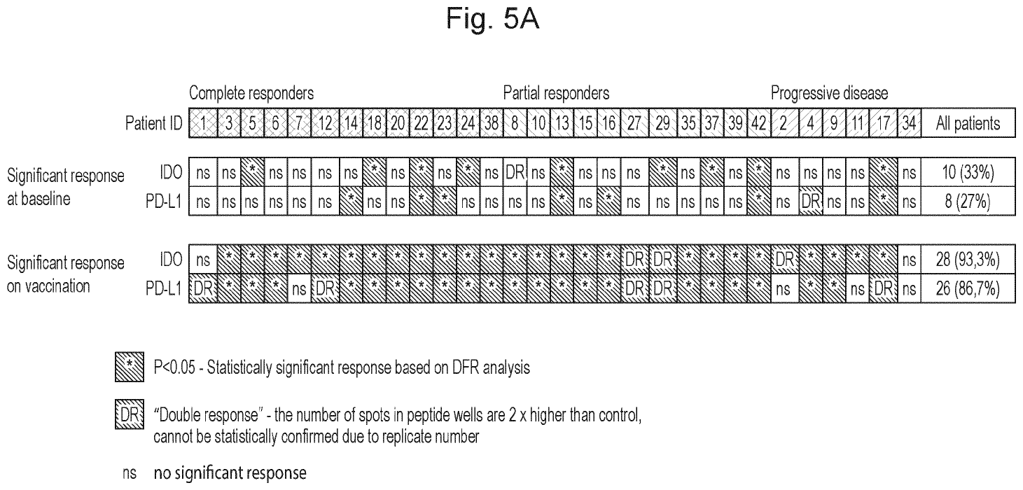

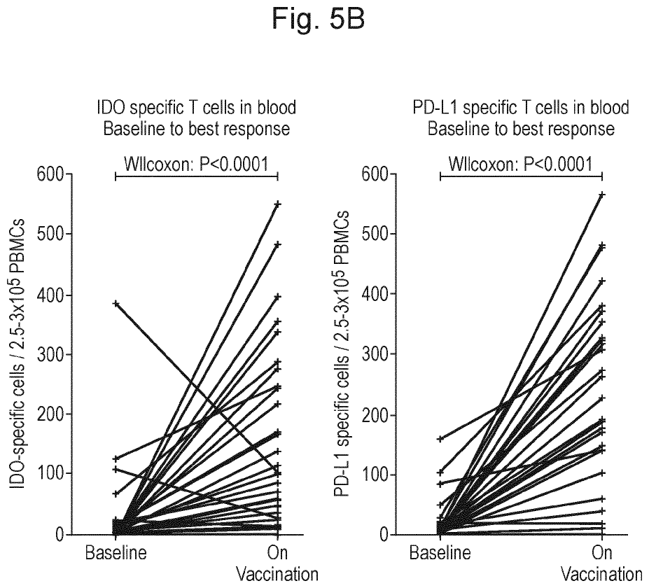

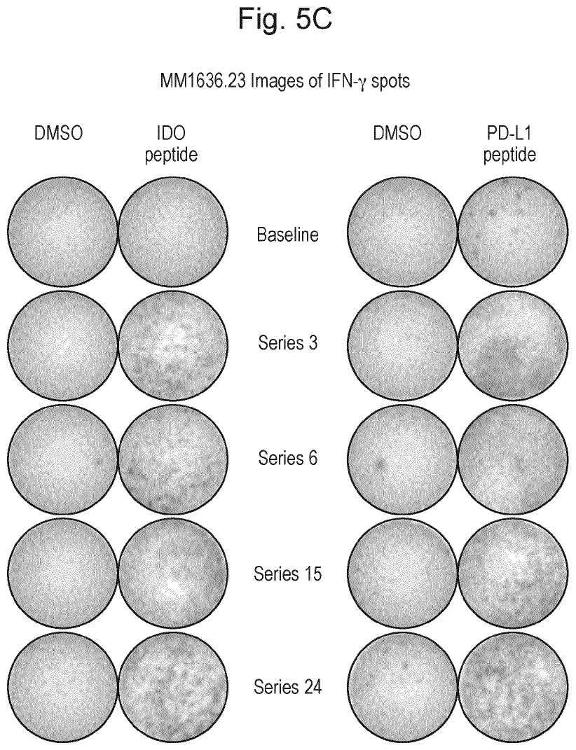

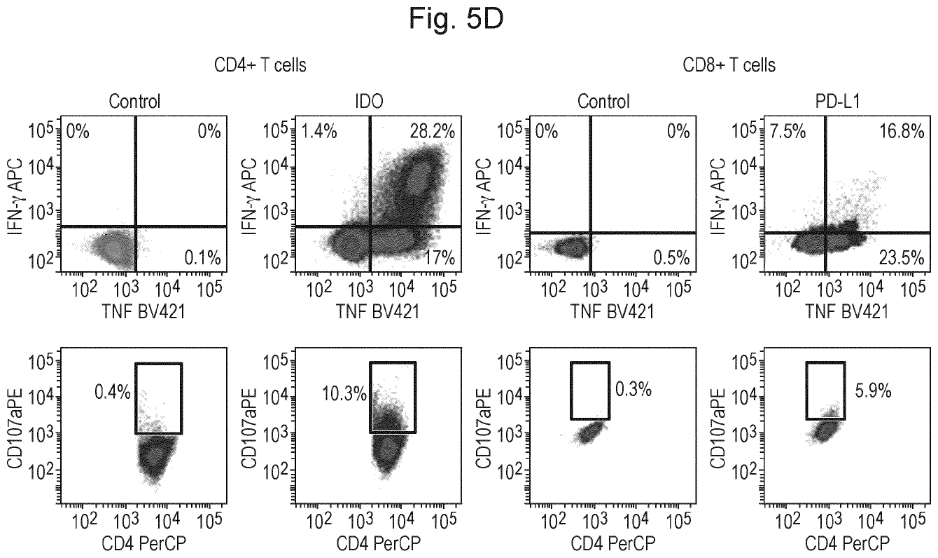

Thirty eligible patients with metastatic melanoma were treated with IDO/PD-L1 vaccine and nivolumab, according to the trial protocol. By investigator review the objective response rate (ORR) reached 80% (CI: 62.7-90.5%), with a majority of 43% (CI: 27.4-60.8%) achieving complete response (CR) and 37% (CI: 20.9-54.5%) reaching partial response (PR) as best overall response (BOR), while 20% experienced progressive disease (PD) according to RECIST 1.1 by investigator review in all patients (n=30), PD-L1+(>1%, (n=17)) and PD-L1− (<1%, n=13)), respectively (FIG. 2A). 95% two-sided confidence intervals were constructed using the Clopper-Pearson method. The ORR among PD-L1 positive (>1% (clone 28.8)) patients (n=17) was 94.1% (CI: 73-99.7%) and in PD-L1 negative patients (n=13) 61.5% (CI: 35.5-82.3%) (FIG. 2A). Objective responses were seen in patients irrespective of HLA genotype. (FIG. 5)

FIG. 2C provides the best change in the sum of target lesion compared with baseline (n=30). Horizontal line at −30 shows threshold for defining objective response in the absence of non-target disease progression or new lesions according to RECIST 1.1. Two patients with 100% reduction in target lesion size have nontarget lesions present. White stars: Six patients have normalization (<10 mm) of FDG-negative lymph nodes and 100% reduction of non-lymph node lesions and are considered CR (green bar). Black star: One patient (MM29) was considered PR (blue bar) though he had not reached −30% in target lesion. The patient had a single measurable 13 mm lung metastasis at baseline and multiple biopsy verified cutaneous metastases on left crus (not detectable on PET/CT at baseline). Best change in target lesion was 10 mm and a post-treatment biopsy from the cutaneous metastases showed no sign of malignancy. Thus, overall, the patient was termed PR.

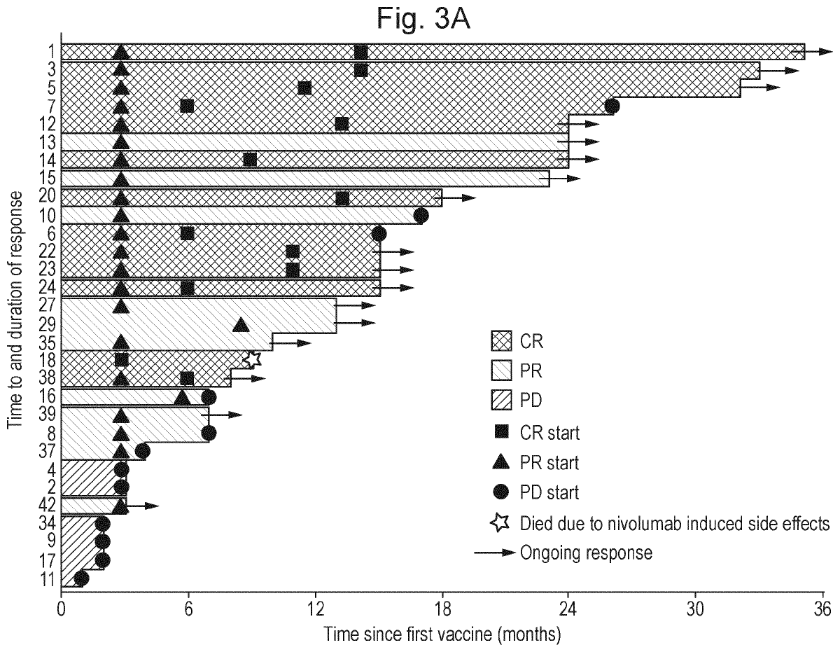

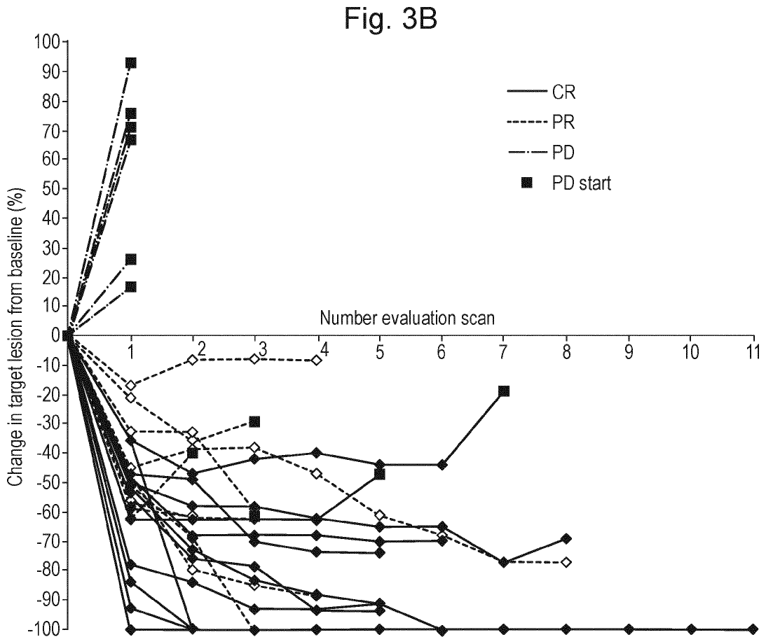

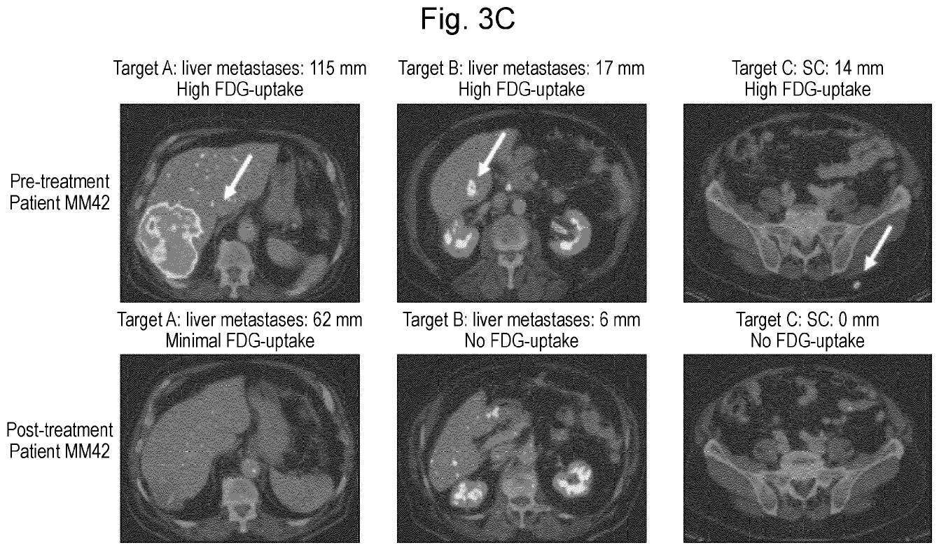

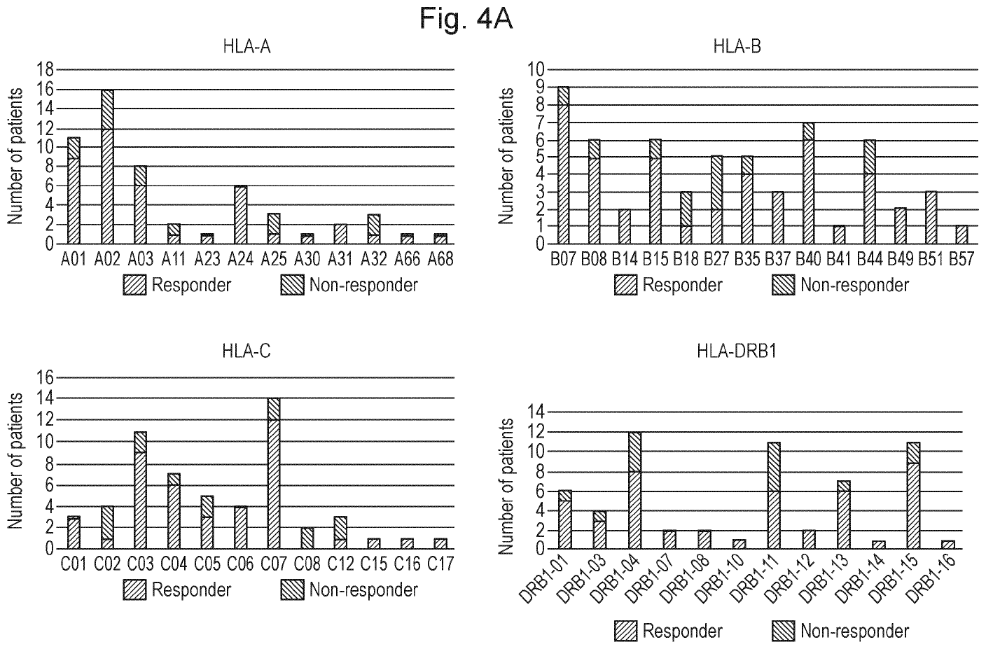

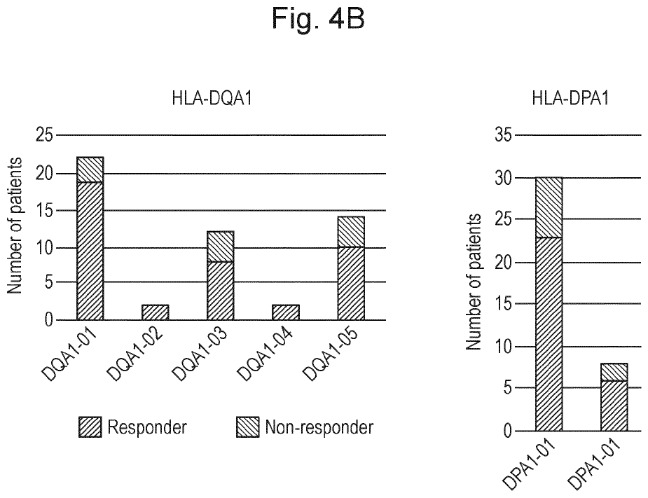

Two of the PR patients did not have confirmation of PR on two consecutive scans. Early onset of response was frequent with 22 of 30 patients having an objective response at 1st evaluation (after 12 weeks on treatment). Median time to PR and CR were 75 days (range 54-256 days) and 327 days (range 73-490 days), respectively. (FIG. 3A-FIG. 3C).

Clinical response data were validated by blinded independent external review, where an ORR of 76.6% (CL: 57.7%-90.1%) was reported with 53.3% achieving CR, 23.3% PR, and 3.3% stable disease. Comparison between investigator review and external review are outlined in Table 4.

TABLE 4
Investigator vs independent central response review (n = 30)

| | Investigator | Independent | Explanation of inconsistencies |
|---|---|---|---|
| Patient ID | review | central review | between RECIST 1.1 response evaluation |
| MM1636.01 | CR | CR | NA |
| MM1636.02 | PD | PD | NA |
| MM1636.03 | CR | CR | NA |
| MM1636.04 | PD | PD | NA |
| MM1636.05 | CR | CR | NA |
| MM1636.06 | CR | CR | NA |
| MM1636.07 | CR | PR | Baseline PET/CT with two mediastinal lymph |
| | | | nodes as target lesions and no non-target |
| | | | lesions. Investigator asses CR due to |
| | | | normalization of both glands and no FDG |
| | | | uptake. Independent reviewer asses PR due to |
| | | | measurable lymph nodes of respectively 11 mm |
| | | | and 11 mm on same scan. |
| MM1636.08 | PR | PR | NA |
| MM1636.09 | PD | PD | NA |
| MM1636.10 | PR | CR | Baseline PET/CT with one target lesion in the |
| | | | lung (16 mm) and non-target lung lesions. |
| | | | Investigator asses PR due to still visible non- |
| | | | target lesions but the target lesion is gone. |
| | | | Independent central reviewer asses CR due to |
| | | | target lesion 0 mm and considers non-target |
| | | | lesions to be non-malignant. |
| MM1636.11 | PD | PD | NA |
| MM1636.12 | CR | CR | NA |
| MM1636.13 | PR | CR | Baseline PET/CT with three target lesions: two |
| | | | mediastinal lymph nodes (41 mm and 26 mm) |
| | | | and a liver metastasis (9 mm) and non-target |
| | | | lesions (lymph nodes). All non-target lesions are |
| | | | gone, and the two lymph node target lesions |
| | | | have normalized and the liver metastases is 3 |
| | | | mm. Investigator asses PR due to FDG uptake |
| | | | in one of the mediastinal lymph nodes and it |
| | | | keeps decreasing in size. Independent reviewer |
| | | | asses as CR due to CT normalization of lymph |
| | | | nodes and asses liver metastases of 3 mm to be |
| | | | non-malignant with no FDG uptake. |
| MM1636.14 | CR | CR | |
| MM1636.15 | PR | CR | Baseline PET/CT with two liver target lesions |
| | | | (11 mm and 12 mm) and non-target lesions |
| | | | consisting of other liver metastases and lymph |
| | | | nodes in the mesorectal fat. All target lesions |
| | | | are 0 and all non-target liver metastases are |
| | | | gone. Investigator asses PR due to continued |
| | | | FDG uptake in a normalized mesorectal lymph |
| | | | node. Independent central review asses CR due |
| | | | to CT normalization of non-target lymph nodes |
| | | | and no liver metastases. |
| MM1636.16 | PR | SD | Investigator measures baseline PET/CT target |
| | | | lesion in liver to be 14 mm while independent |
| | | | central review measures it to be 12 mm. No |
| | | | non-target lesions. Investigator review measures |
| | | | lesion to 9 mm while independent central |
| | | | review measure it to 11 mm and therefore asses |
| | | | PR vs SD. |
| MM1636.17 | PD | PD | NA |
| MM1636.18 | CR | CR | NA |
| MM1636.20 | CR | CR | NA |
| MM1636.22 | CR | CR | NA |
| MM1636.23 | CR | CR | NA |
| MM1636.24 | CR | CR | NA |
| MM1636.27 | PR | PR | NA |
| MM1636.29 | PR | CR | Baseline PET/CT with one target lesion in right |
| | | | lung (13 mm) and no non-target lesions. At |
| | | | baseline cutaneous metastases on left crus, |
| | | | biopsy verified but not detectable on PET/CT |
| | | | scan. Investigator asses best decrease in target a |
| | | | to be 10 mm and a biopsy from cutaneous |
| | | | metastases on crus is without malignancy. Is |
| | | | therefore assessed to be PR. Independent |
| | | | reviewer asses target lesion to decrease to 0 mm |
| | | | and therefore asses patient to be in CR. |
| MM1636.34 | PD | PD | NA |
| MM1636.35 | PR | PR | NA |
| MM1636.37 | PR | PR | NA |
| MM1636.38 | CR | CR | NA |
| MM1636.39 | PR | PR | NA |
| MM1636.42 | PR | PR | NA |

To examine whether the observed very high response rate was attributable to nivolumab or to the vaccine, clinical response information of a matched historical control group using the Danish Metastatic Melanoma Database (DAMMVED) was retrieved from contemporaneously treated stage IJI-IV melanoma patients who received αLPD1 monotherapy (Ellebaek, E. et al. The Danish Metastatic Melanoma Database (DAMMED): A nationwide platform for quality assurance and research in real-world data on medical therapy in Danish melanoma patients. Submiss. (2021)). Patients were matched with the exact same combination variable according to age, gender, PD-L1 status, BRAF-status, LDH level and M-stage (M1d were excluded from the control group (no patients with brain metastasis)). Exact matched controls were identified for 29 patients and estimates for treatment effects were calculated by weighted logistic regression analyses and weighted cox proportional hazard model. Odds ratios (OR), response rates and their corresponding 95% confidence intervals were extracted from the regression model. All p-values were two-sided and p-values below 0.5 were considered statistically significant. The ORR of 79.3% (CI: 61.0-90.4%) observed in MM1636 was significantly higher (p<0.0012) compared 156 to the matched control group where an ORR of 41.7% (CI: 31.0-53.3%) was reached. Furthermore, of the 29 patients in MM1636, a significantly (p<0.0017) higher percentage of 41.4% (CI: 25.2-59.6%) patients achieved CR in MM1636, compared to 12% (CI: 6.3-21.6%) in the matched historical control group. ORR and CRR in the matched historical control group are comparable to patients treated in randomized phase III pivotal trials with αPD1 monotherapy (FIG. 2B).

### Combination of the IDO/PD-L1 Vaccine and Nivolumab Leads to Prolonged Progression-Free Survival

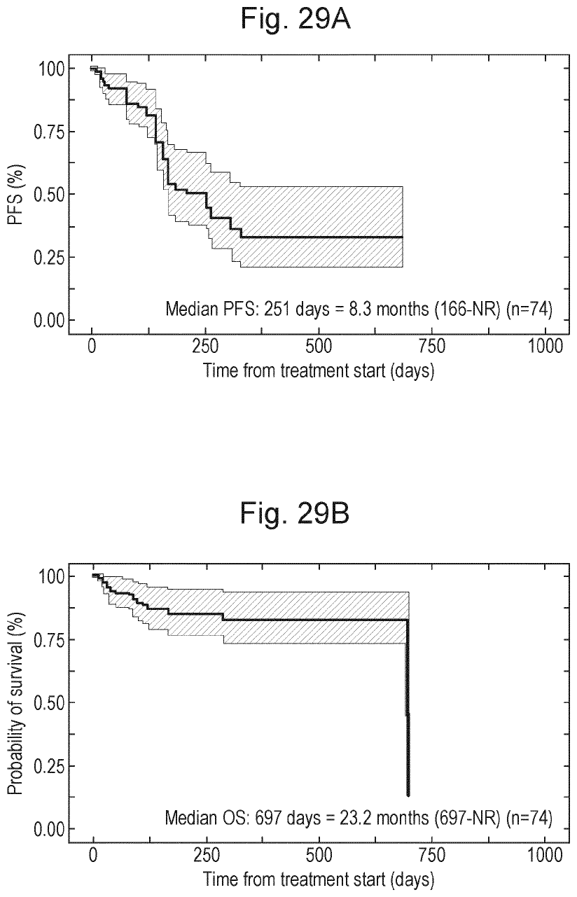

At data cut-off, the median duration of response had not been reached, with 87% of all responding patients being progression-free at 12 months (FIG. 2D). Patients were followed for up to 35 months with a median follow-up time of 22.9 months (CI: 14.9-26.2 months). Overall survival (OS) and progression-free survival (PFS) were calculated from first day of treatment to death or progression, or to the date of the last follow-up (Oct. 5, 2020). The median PFS (mPFS) for all treated patients was 26 months (CI: 15.4-69) and was not reached for responding patients (FIG. 2E). The median OS was not reached at the data cut-off. OS at 12 months was 81.6% (CI: 61.6-92%) (FIG. 2F). One patient (MM18) in CR died from nivolumab-related severe adverse events, the remaining deaths observed were due to metastatic melanoma. For comparison, the mPFS was 8.3 months (CI: 5.5-NR) in the matched historical control group (n=74), while the mOS 183 was 23.2 months (CI: 23.2-NR) (FIG. 29)

The Combination of IDO/PD-L1 Vaccine and Nivolumab was Safe, and Systemic Side Effects were Comparable to Nivolumab Monotherapy

Treatment-related adverse events (AEs) are listed for all 30 patients in Table 5. The table sums all treatment related AEs. All percentages together total more than 100% due to more AEs per patient. Patients might have had multiple AEs at the same time point. Patient MM18 died due to urosepsis and multi organ failure and severe hyponatremia (grade 3). This patient had experienced many treatment related side effects up to her death with grade 3 colitis, grade 2 pneumonitis, grade 3 arthralgia, grade 2 vasculitis and grade 2 nivolumab induced infusion allergic reaction. Further, patient MM18 had symptoms of myocarditis at time of death with highly elevated troponin I and a bedside ECCO showed an ejection fraction of 15%, which at baseline was 60%, but the myocarditis was never confirmed pathologically due to missing autopsy.

Common treatment-related grade 1-2 toxicities were fatigue (47%), rash (47%), arthralgia (30%), diarrhoea (23%), nausea (23%), dry skin (20%), pruritus (20%), infusion reaction (17%), xerostomia (17%) and myalgia (17%). Four patients (13%) experienced grade 3-4 adverse events, one patient with grade 3 maculopapular rash (MM01), one patient with grade 3 adrenal insufficiency (MM06), and one with grade 3 arthralgia (MM22).

TABLE 5
Treatment related adverse events (n = 30)

| | Grade 1-2 | | Grade 3-4 | |
|---|---|---|---|---|
| Skin | | | | |
| Rash | 14 | 47% | 1 | 3% |
| Dry skin | 6 | 20% | | |
| Pruritus | 6 | 20% | | |
| Vitiligo | 4 | 13% | | |
| Vasculitus | | 3% | | |
| Gastrointestinal | | | | |
| Diarrhoea | 7 | 23% | 1 | 3% |
| Constipation | 1 | 3% | | |
| Abdominal pain | 1 | 3% | | |
| Colitis | 1 | 3% | 1 | 3% |
| Respiratory | | | | |
| Pneumonitis | 3 | 10% | | |
| Pleural effusion | 1 | 3% | | |
| Dyspnea | 4 | 13% | | |
| Endocrinic | | | | |
| Adrenal insufficiency | 2 | 7% | 1 | 3% |
| Hypophysitis | 2 | 7% | | |
| hypothyroidisme | 1 | 3% | | |
| Hyperthyrpodisme | 2 | 7% | | |
| Musculosceletal | | | | |
| Arthralgia | 9 | 30% | 2 | 7% |
| Myalgia | 5 | 17% | | |
| Laboratory tests | | | | |
| Increased amylase | 6 | 20% | | |
| Increase ALAT | 3 | 10% | | |
| Hyponatremia | | | 1 | 3% |
| Infusion reaction | 5 | 17% | | |
| Other | | | | |
| Nausea | 7 | 23% | | |
| Pruritus | 6 | 20% | | |
| Xerostomia | 5 | 17% | | |
| Stomatitis | 2 | 7% | | |
| Nasal congestion | 2 | 7% | | |
| Periorbital oedema | 1 | 3% | | |
| Dry eyes | 1 | 3% | | |
| Peripheral neuropathy | 1 | 3% | | |
| Hearing imparied | 1 | 3% | | |
| Vaccine related | | | | |
| Injection site reaction | 23 | 77% | | |
| Granuloma at injection site | 19 | 63% | | |
| Redness at injection site | 6 | 20% | | |
| Pain at injection site | 4 | 13% | | |
| Pruritus at injection site | 4 | 13% | | |
| Myalgia at injection site | 1 | 3% | | |

Patient MM18 died due to urosepsis with multi-organ failure and severe hyponatremia. This patient had experienced multiple immune-related AEs with grade 3 colitis, grade 2 pneumonitis, grade 3 arthralgia, grade 2 vasculitis, and grade 2 nivolumab infusion related allergic reaction. Additionally, patient MM18 had symptoms of myocarditis at the time of death with highly elevated troponin I. Bedside echocardiography showed an ejection fraction of 15%, which 188 at baseline was 60%, but an autopsy was not conducted, and the myocarditis was therefore not confirmed pathologically.

Patient MM06 had received first-line treatment with ipilimumab before entering the trial and was on substitution corticosteroids at the time of inclusion. Adrenal insufficiency was aggravated by an erysipelas infection with high fever, reaching CTCAE grade 3 level, resolving quickly after appropriate antibiotic therapy was initiated.

As expected, local side effects were common with 77% of the patients who developed injection site reactions. These reactions were classified as 63% granulomas, 20% redness, 13% pain and 13% pruritus at injection site. All of these local reactions were grade 1-2, most likely related to montanide adjuvant and typically transient. However, two patients (MM07 and MM20) requested to discontinue vaccination after 8 and 11 injections, respectively due to granulomas, tenderness, and pain that limited instrumental activities of daily living but continued nivolumab. FIG. 6 provides images of injection site reaction in patient MM20 (CR) after 11 vaccination show redness, rash, and granuloma at injection site.

Vaccine-Specific Responses in Blood were Detected in the Majority of Patients with Durable Responses Post-Vaccination

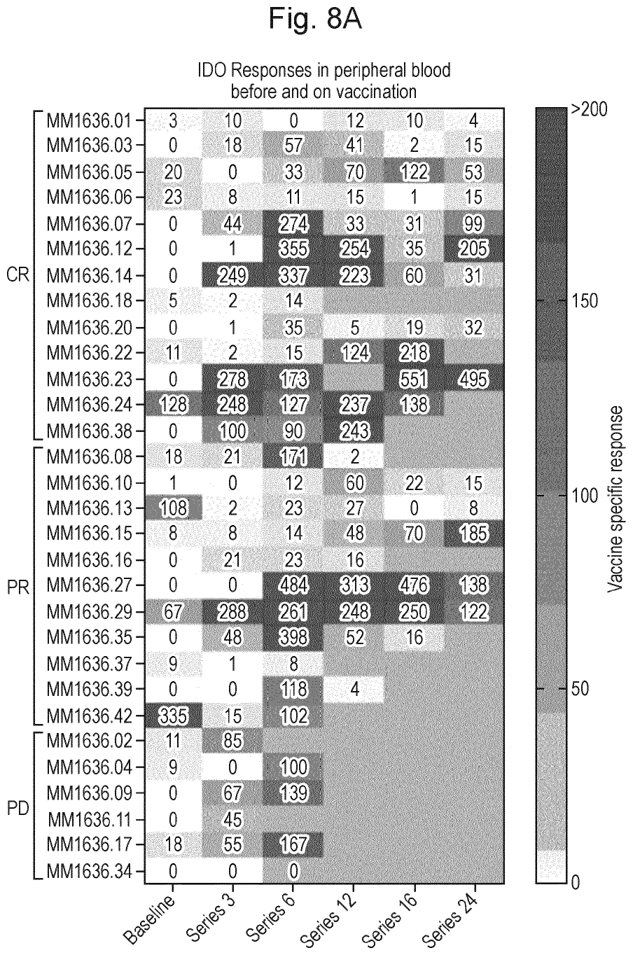

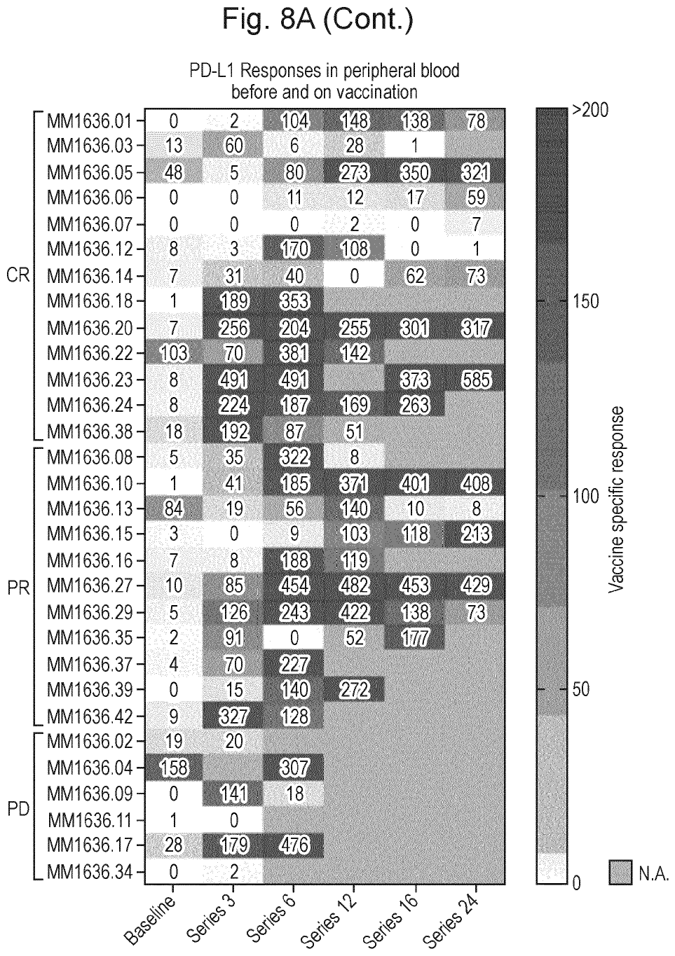

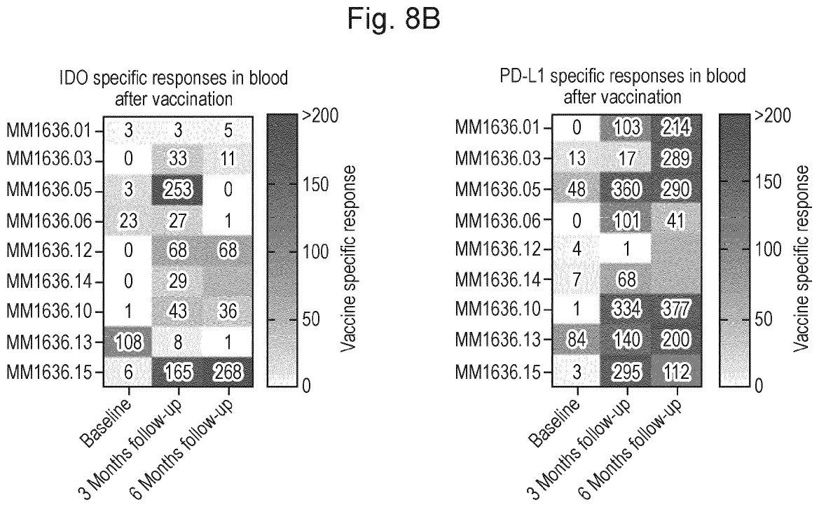

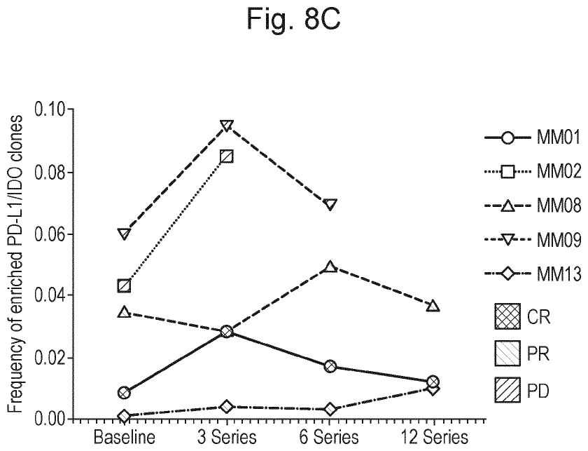

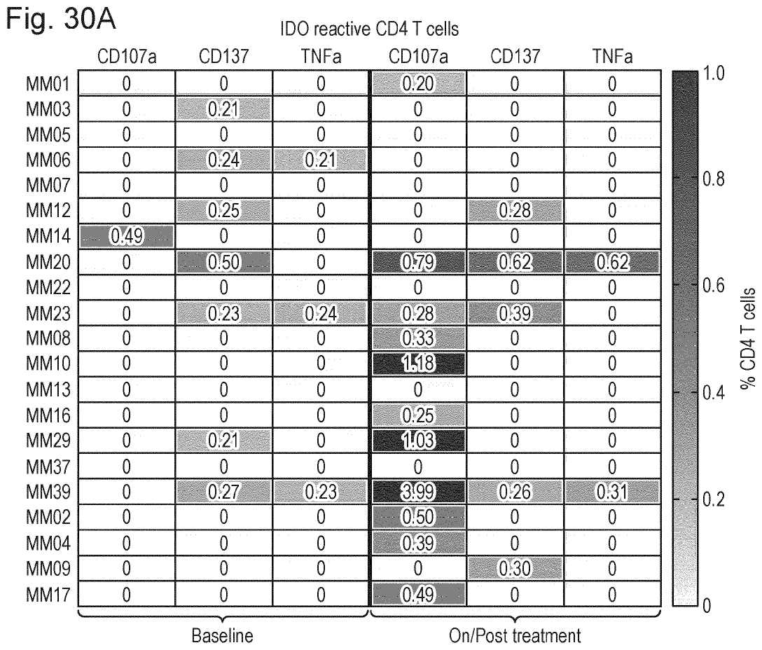

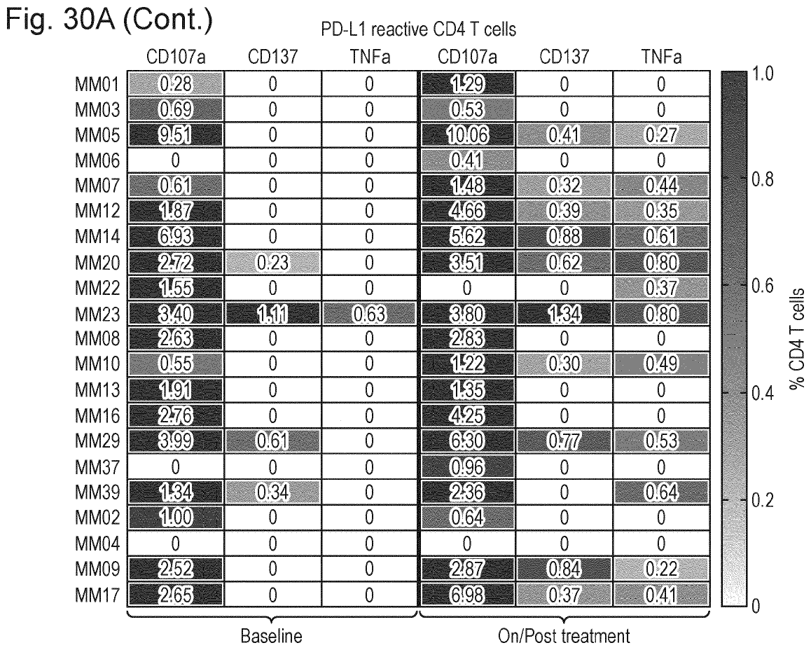

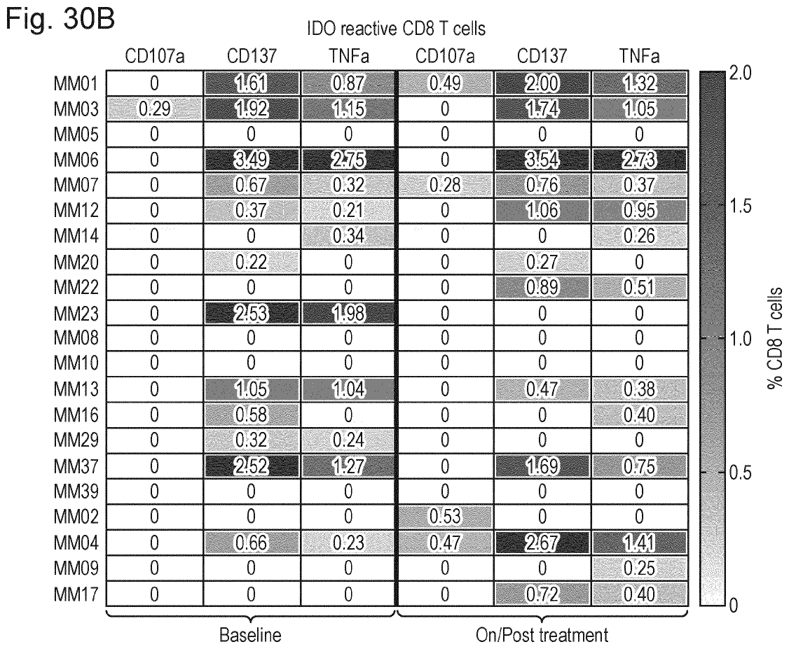

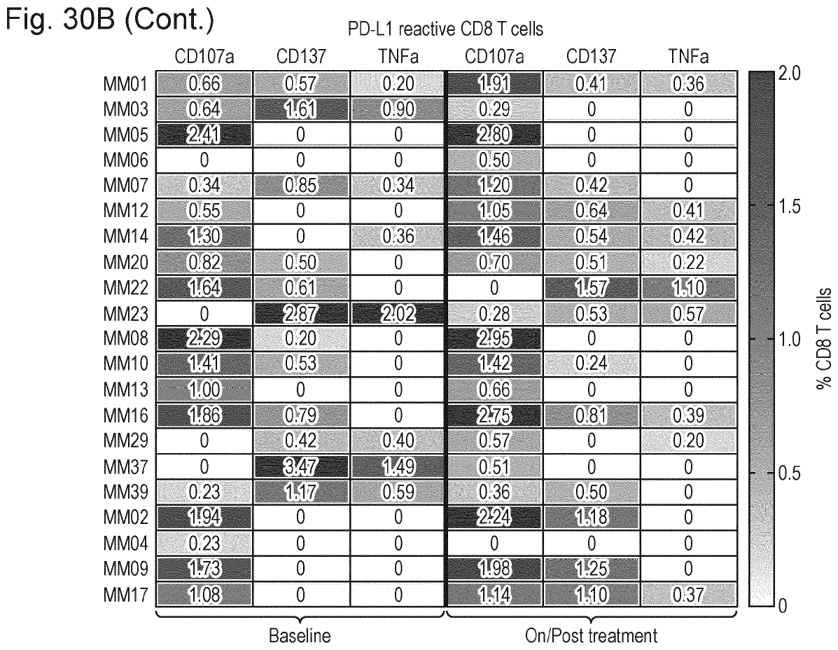

All 30 patients were assessed for the presence of vaccine-specific responses in peripheral blood mononuclear cells (PBMCs) before, on and after vaccination in vitro. Pre-vaccine IDO responses were detectable in 10 (33%) patients, while pre-vaccine PD-L1 responses were detectable in 8 patients (27%), overlapping (both IDO and PD-L1) pre-vaccine responses were present in 4 (13.3%). During vaccination, an increase of IDO specific T-cells or PD-L1 specific T-cells in blood was observed in 27 (90%) and 25 (83%) patients, respectively. 93% of patients had an increase in either PD-L1 or IDO responses on vaccination (FIG. 5A). Responses were calculated as the difference between average numbers of spots in wells stimulated with IDO or PD-L1 peptide (triplicates) and corresponding control (DMSO) and statistical analyses of Elispot responses were performed using distribution free resampling method (Moodie et al.). DR: Not statistically confirmed response due to replicate number but number of spots in peptide wells are two times higher than control wells (DMSO). NS: No significant response and no DR. For detailed overview of responses at serial time points on vaccination see FIG. 6A. A significant (p<0.0001) median increase from baseline to post-vaccine response was observed for both IDO and PD-L1 (at different time-points on treatment), confirming that vaccine-specific immune responses were induced in patients regardless of clinical response. (FIG. 5B). Vaccination responses were selected from “best” Elispot response at different time points for each patient (series 3, 6, 12, 18 or 24) during vaccination. Wilcoxon matched-pairs signed-rank test was used to compare responses to IDO or PD-L1 vaccine peptides between baseline and later time points. The immune responses fluctuated in blood over time (FIG. 8A). Increases in IDO and PD-L1 responses in the peripheral blood was also detectable directly ex vivo across the different clinical response groups (FIG. 30).

Sustained vaccine-specific responses were observed 3 and 6 months after the last vaccine, indicating induction of memory responses in 9 patients with clinical treatment response who surpassed follow up at data lock (FIG. 8B). Importantly, PD-L1 and IDO specific responses were observed irrespective of HLA-genotype. (Table 6).

TABLE 6
HLA-gentotype on all treated patients (n = 30)

| | | | | HLA- | HLA- | HLA- | HLA- |
|---|---|---|---|---|---|---|---|
| Patient ID | HLA-A | HLA-B | HLA-C | DRB1 | DQA1 | DPA1 | DQB1 |
| MM1636.01 | A01 A25 | B08 B18 | C07 C12 | 03 15 | 01 05 | 01 01 | 02 06 |
| MM1636.02 | A01 A03 | B27 B44 | C02 C05 | 04 11 | 03 05 | 01 01 | 03 03 |
| MM1636.03 | A01 A02 | B08 B40 | C03 C07 | 03 13 | 01 05 | 01 02 | 02 06 |
| MM1636.04 | A03 A25 | B07 B18 | C07 C12 | 04 15 | 01 03 | 01 02 | 03 06 |
| MM1636.05 | A01 A03 | B08 B49 | C07 C07 | 03 13 | 01 05 | 02 02 | 02 06 |
| MM1636.06 | A02 A68 | B15 B37 | C03 C06 | 01 13 | 01 01 | 01 02 | 05 06 |
| MM1636.07 | A23 A31 | B07 B44 | C04 C07 | 11 15 | 01 05 | 01 01 | 03 06 |
| MM1636.08 | A02 A26 | B35 B51 | C04 C05 | 11 13 | 01 05 | 01 01 | 03 06 |
| MM1636.09 | A02 A32 | B15 B44 | C03 C05 | 04 11 | 03 05 | 01 01 | 03 03 |
| MM1636.10 | A03 A24 | B07 B51 | C07 C15 | 15 16 | 01 01 | 01 02 | 05 06 |
| MM1636.11 | A02 A25 | B18 B27 | C02 C12 | 04 15 | 01 03 | 01 01 | 03 06 |
| MM1636.12 | A03 A66 | B15 B37 | C03 C06 | 04 10 | 01 03 | 01 01 | 03 05 |
| MM1636.13 | A01 A02 | B08 B40 | C03 C07 | 04 13 | 01 03 | 01 01 | 03 06 |
| MM1636.14 | A02 A03 | B07 B51 | C01 C07 | 01 11 | 01 05 | 01 01 | 03 05 |
| MM1636.15 | A24 A32 | B40 B44 | C03 C05 | 04 11 | 03 05 | 01 01 | 03 03 |
| MM1636.16 | A03 A31 | B07 B27 | C02 C07 | 04 12 | 03 05 | 01 01 | 03 03 |
| MM1636.17 | A02 A30 | B14 B27 | C02 C08 | 01 11 | 01 05 | 01 01 | 03 05 |
| MM1636.18 | A02 A02 | B27 B40 | C01 C03 | 01 13 | 01 01 | 01 01 | 05 06 |
| MM1636.20 | A24 A24 | B35 B44 | C04 C16 | 07 08 | 02 04 | 01 02 | 02 04 |
| MM1636.22 | A01 A02 | B07 B49 | C07 C07 | 08 15 | 01 04 | 01 01 | 04 06 |
| MM1636.23 | A03 A03 | B07 B07 | C07 C07 | 01 15 | 01 01 | 01 01 | 05 06 |
| MM1636.24 | A02 A02 | B15 B44 | C04 C05 | 01 04 | 01 03 | 01 01 | 03 05 |
| MM1636.27 | A02 A02 | B40 B41 | C03 C17 | 04 15 | 01 03 | 01 01 | 03 06 |
| MM1636.29 | A01 A11 | B07 B08 | C07 C07 | 04 04 | 03 03 | 01 01 | 03 03 |
| MM1636.34 | A01 A11 | B08 B35 | C04 C07 | 03 11 | 05 05 | 01 02 | 02 03 |
| MM1636.35 | A02 A02 | B15 B40 | C01 C03 | 11 12 | 05 05 | 01 01 | 03 03 |
| MM1636.37 | A01 A24 | B35 B37 | C04 C06 | 11 15 | 01 05 | 01 02 | 03 06 |
| MM1636.38 | A01 A24 | B35 B57 | C04 C06 | 07 14 | 01 02 | 01 02 | 02 05 |
| MM1636.39 | A02 A24 | B15 B15 | C03 C03 | 04 15 | 01 03 | 01 01 | 03 06 |
| MM1636.42 | A01 A02 | B07 B07 | C07 C07 | 15 15 | 01 01 | 01 01 | 06 06 |

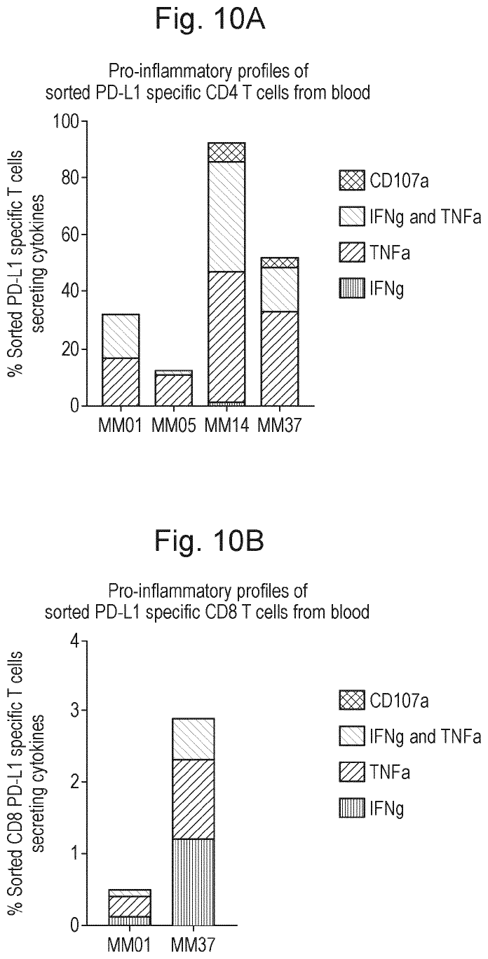

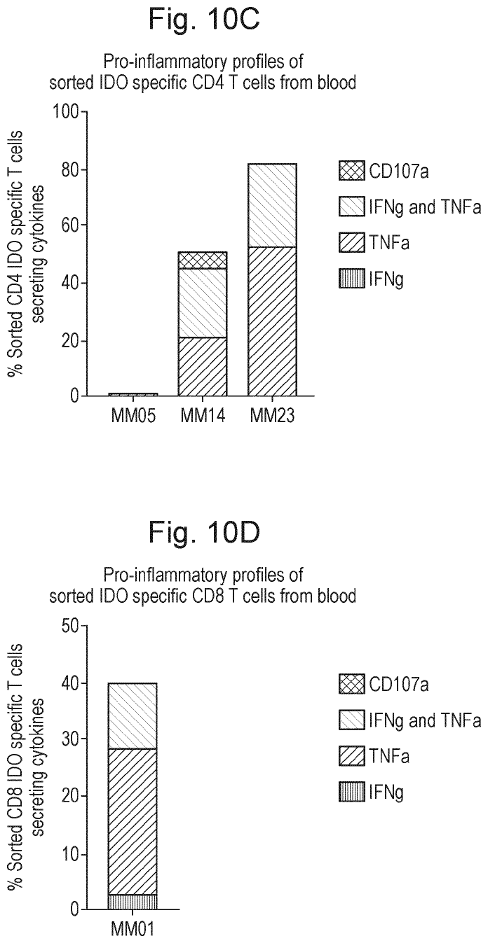

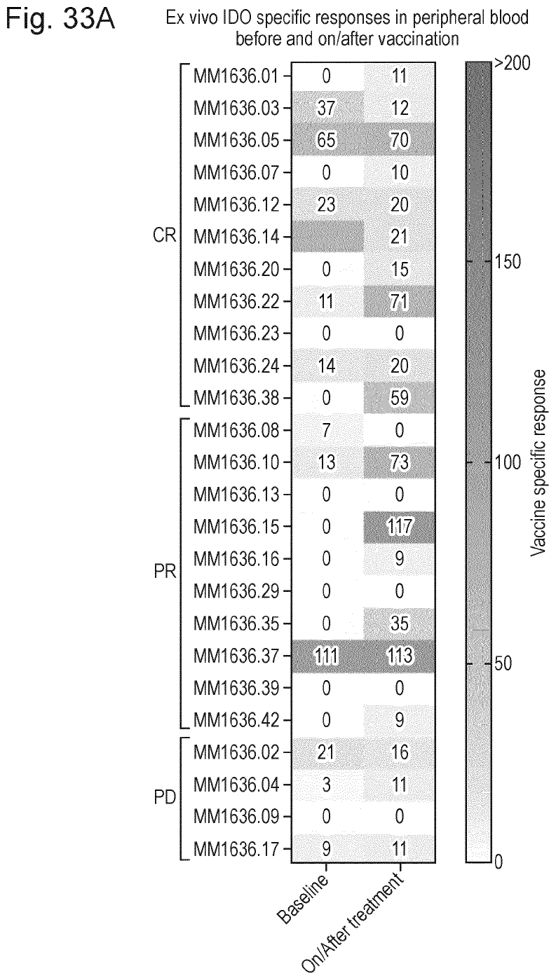

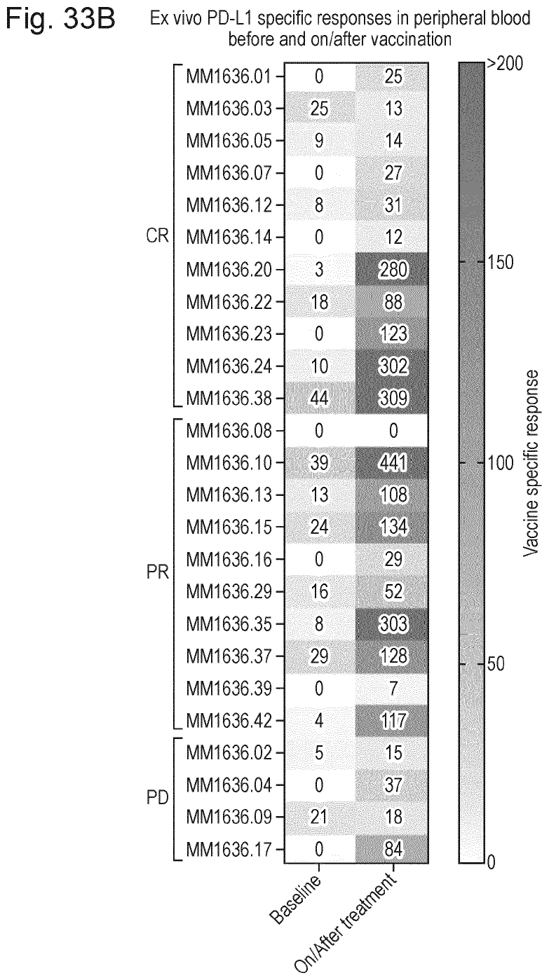

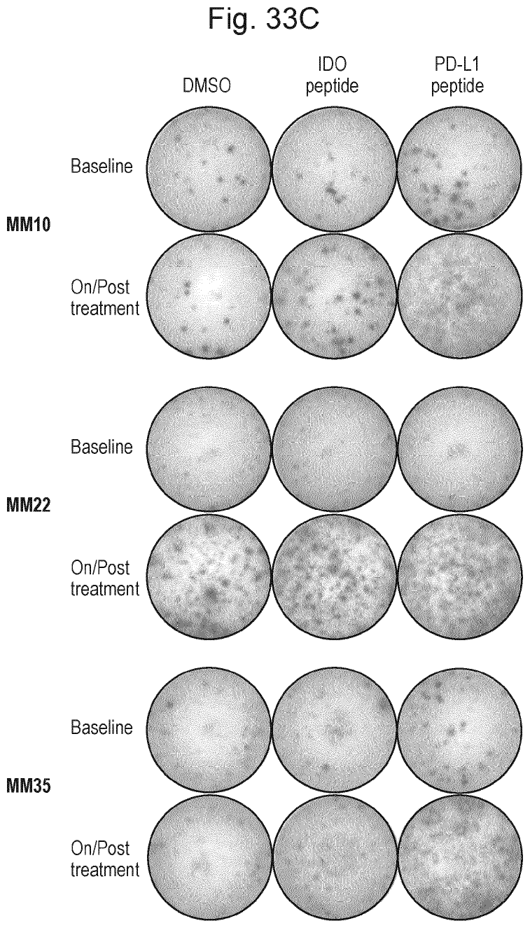

To verify the functionality of vaccine-induced T cells, IDO or PD-L1specific T-cells were isolated from peripheral blood mononuclear cells (PBMCs) from 5 patients. Phenotypic characterization with flow cytometry revealed that the isolated vaccine-specific T-cells consisted both of CD4+ and CD8+ T cells. Also, both IDO and PD-L1 specific CD4+ and CD8+ T-cells showed pro-inflammatory properties, as they expressed the cytolytic marker CD107a and secreted IFN-γ and TNF-αL cytokines (FIG. 5D FIGS. 10A, 10B, 10C, and 10D). Interestingly, vaccine specific CD4+ and CD8+ T cell responses were detected in peripheral blood ex vivo (FIG. 33 and FIG. 30). A significant increase in overall percentage of CD107α˜, CD137, and TNFα expression in response to peptide stimulation in post-treatment PBMC samples compared to baseline was observed, further confirming the expansion and the diverse signature of vaccine specific T cells (FIG. 30).

### Vaccine Specific Responses Detected in the Skin at Vaccination Site

To investigate whether vaccine-specific T-cells have the potential to migrate to peripheral tissue, delayed-type hypersensitivity (DTH) was performed after 6 cycles of treatment on fifteen patients to assess the presence of vaccine-reactive T-cells in the skin. Table 7 display an overview of skin infiltrating lymphocyte (SKILS) cultures.

TABLE 7
Overview of DTH injections and Skin infiltrating T cells

| | | | SKILS | SKILS |
|---|---|---|---|---|
| | | SKILS | grown | grown |
| | | grown | from | from |
| | | from | PD-L1 | IDO |
| Patient ID | DTH injections | mix | alone | alone |
| MM1636.01 | mix of PD-L1/IDO * 3 | yes | NA | NA |
| MM1636.03 | mix of PD-L1/IDO * 3 | yes | NA | NA |
| MM1636.04 | mix of PD-L1/IDO * 3 | yes | NA | NA |
| MM1636.05 | mix of PD-L1/IDO * 3 | no | NA | NA |
| MM1636.06 | mix of PD-L1/IDO * 3 | yes | NA | NA |
| MM1636.08 | PD-L1 alone, IDO alone | no | NA | NA |
| | and mix of PD-L1/IDO | | | |
| MM1636.10 | PD-L1 alone, IDO alone | yes | yes | yes |
| | and mix of PD-L1/IDO | | | |
| MM1636.14 | PD-L1 alone, IDO alone | yes | yes | no |
| | and mix of PD-L1/IDO | | | |
| MM1636.15 | PD-L1 alone, IDO alone | yes | yes | yes |
| | and mix of PD-L1/IDO | | | |
| MM1636.16 | PD-L1 alone, IDO alone | yes | yes | no |
| | and mix of PD-L1/IDO | | | |
| MM1636.20 | PD-L1 alone, IDO alone | yes | yes | no |
| | and mix of PD-L1/IDO | | | |
| MM1636.22 | PD-L1 alone, IDO alone | yes | yes | no |
| | and mix of PD-L1/IDO | | | |
| MM1636.23 | PD-L1 alone, IDO alone | yes | yes | no |
| | and mix of PD-L1/IDO | | | |
| MM1636.24 | PD-L1 alone, IDO alone | yes | yes | no |
| | and mix of PD-L1/IDO | | | |
| MM1636.27 | PD-L1 alone, IDO alone | yes | no | yes |
| | and mix of PD-L1/IDO | | | |

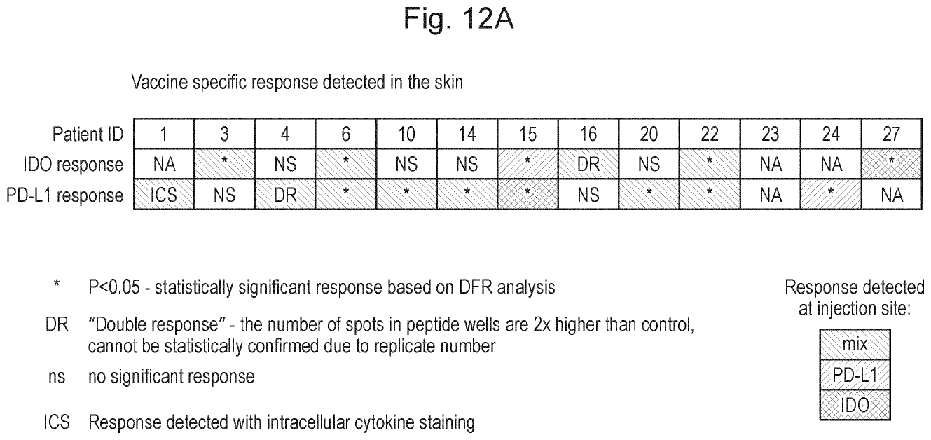

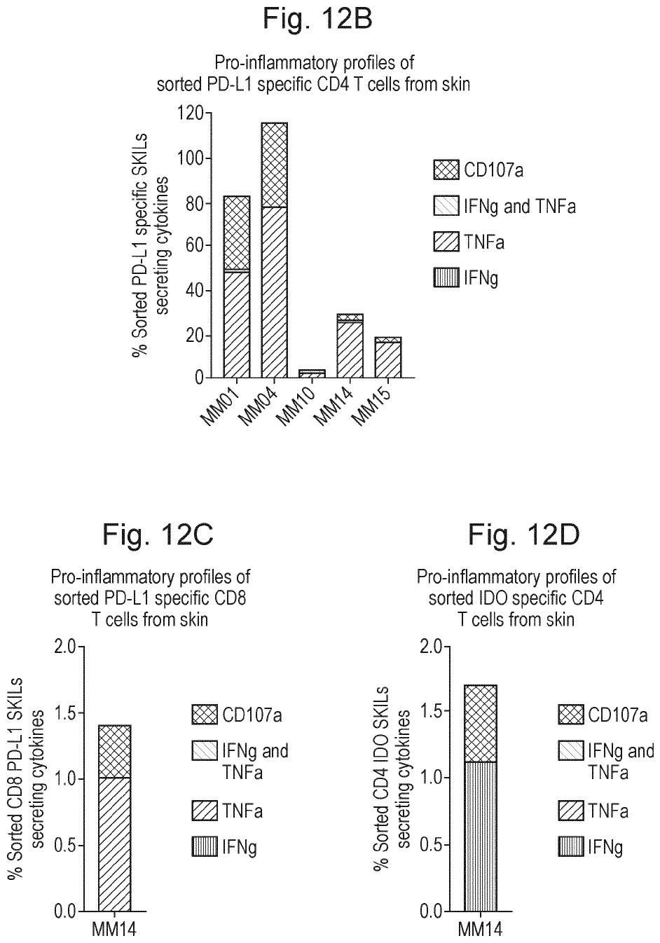

IDO specific T-cells were shown in the skin from 6 of 10 patients and PD-L1 specific T cells in 9 of 11 patients. (FIG. 12A). SKILs were grown from DTH injection with either IDO peptide, PD-L1 peptide or a mix as presented by different blue colours. Responses were calculated as the difference between average numbers of spots in wells stimulated with IDO or PD-L1 peptide (triplicates) and corresponding control (DMSO) and statistical analyses of Elispot responses were performed using distribution-free resampling method (Moodie et al.). DR: Not statistically confirmed response due to replicate number but number of spots in peptide wells are two times higher than control wells (DMSO). NS: No significant response and no DR Intracellular cytokine staining was performed on SKILs from five patients after stimulation with either PD-L1 or IDO. The major fraction detected was CD4+ peptide-reactive T-cells that secreted TNF-α, upregulated CD107a and a minor fraction also secreted IFN-7. In one patient, CD8+PD-L1 reactive T-cells were detected. (FIGS. 2B, 12C, and 12D).

### Direct Recognition of IDO- and PD-L1 Target Cells by Vaccine-Induced T Cells

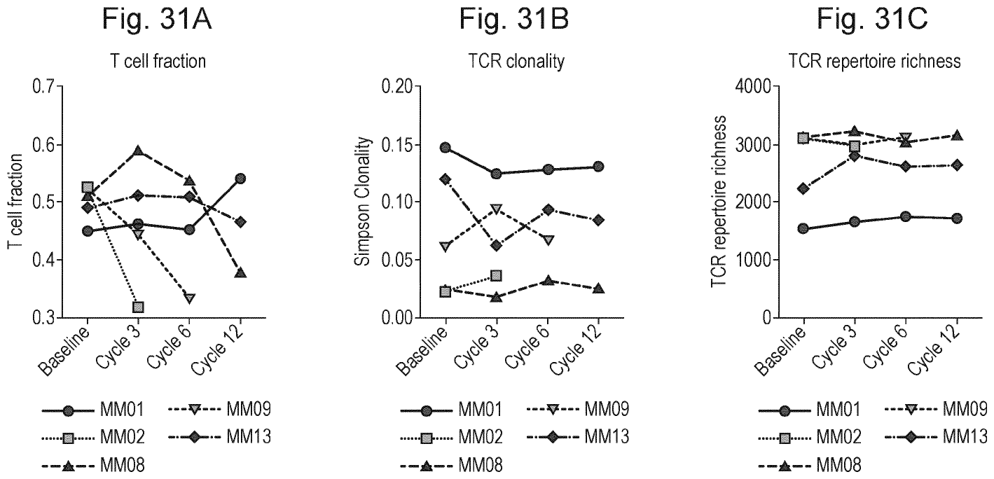

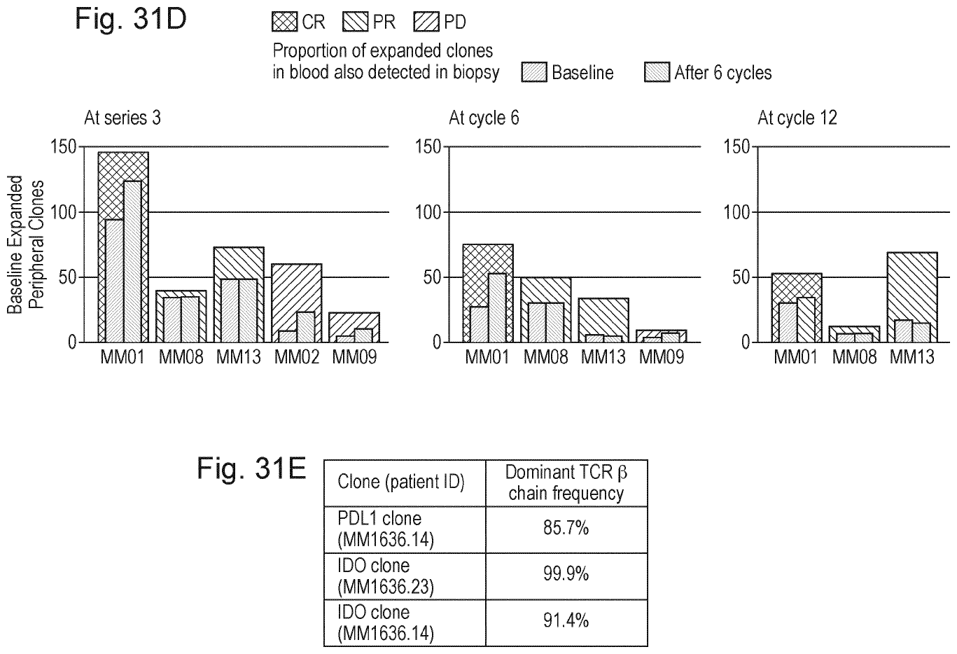

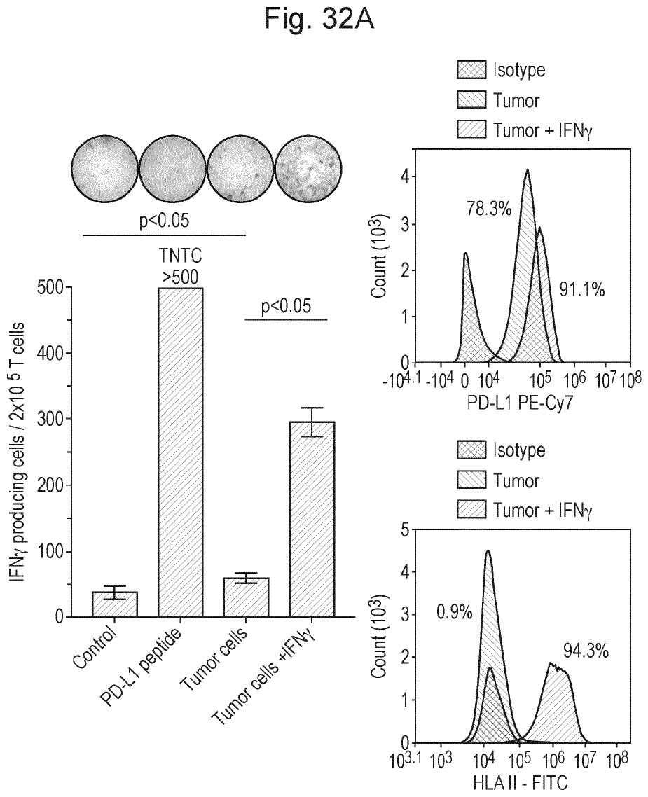

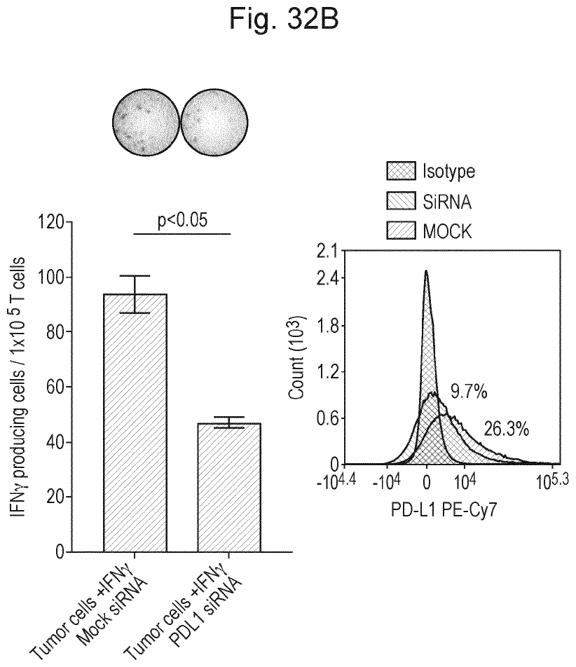

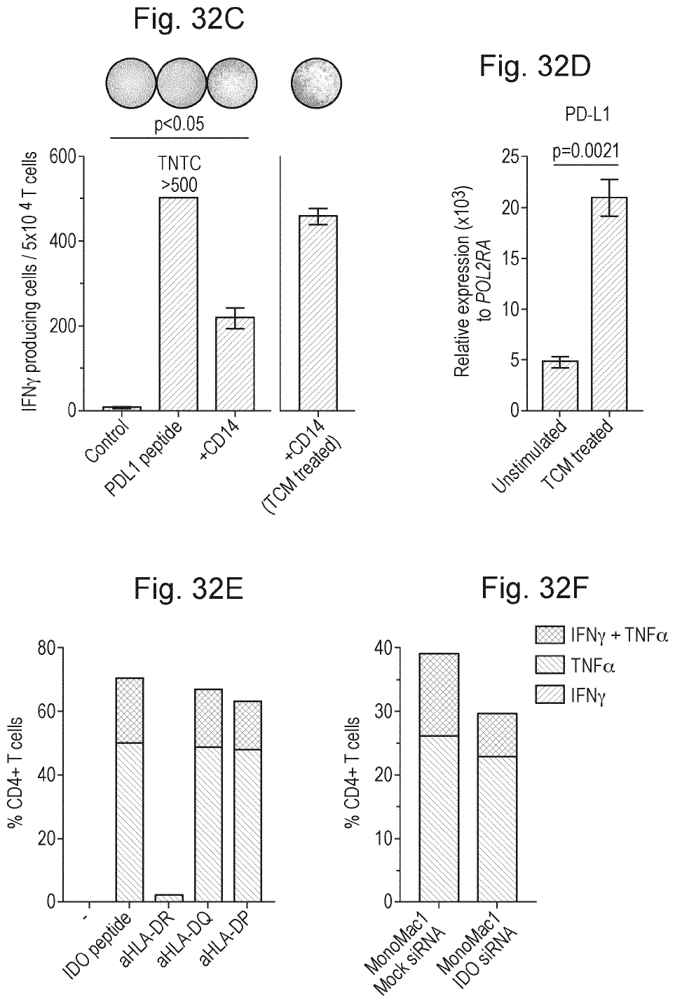

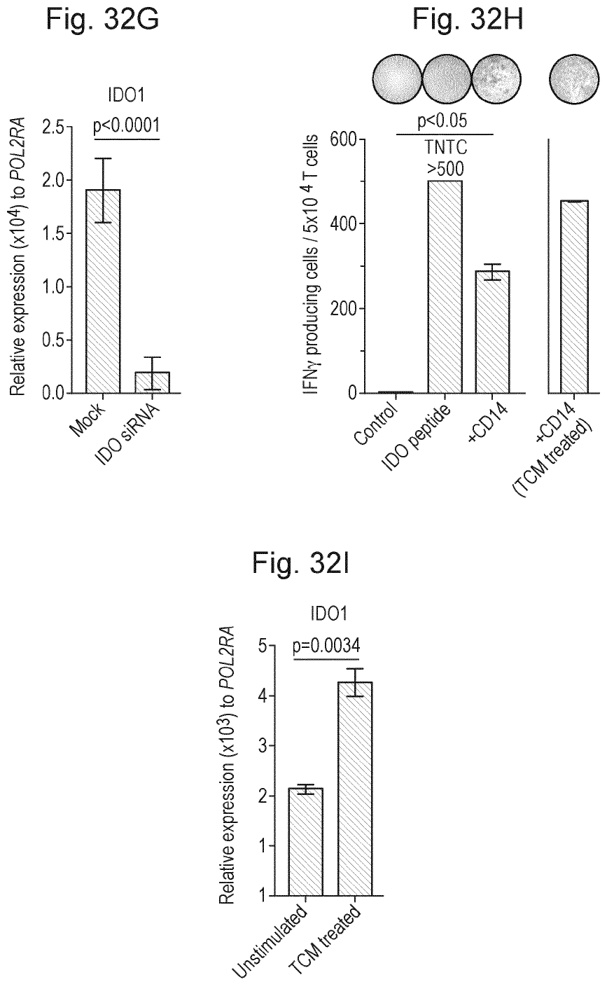

To confirm the functionality of vaccine-expanded T cells, vaccine specific T cell clones (clonal purity confirmed by T cell receptor (TCR) sequencing) were isolated and expanded from patient PBMCs (FIG. 31E). It was shown that PD-L1 specific T cells were able to recognise PD-L1+ autologous tumour cells in a PD-L1 expression dependant manner if cancer cells also expressed HLA-II (FIGS. 32A and 32B). Similarly, HLA-DR-restricted IDO specific CD4+ T-cell clone was able to recognise an HLA-DR-matched IDO-expressing model cell line MonoMac1 in an IDO-expression dependant manner (FIG. 32E-G). As previously described, IDO and PD-L1 specific T cell mode of action is not limited to targeting only cancer cells. It was shown that vaccine specific T cell clones were also reacting against PD-L1 and IDO-expressing autologous immune cells (FIGS. 32C and 32H). To provide myeloid cells with a tumour-associated phenotype, isolated CD14+ myeloid cells were treated with tumour conditioned media (TCM) derived from an established autologous tumour cell line. It was observed that such TCM treated CD14+ cells had an increased expression of PD-L1 and IDO and were effectively recognized by autologous PD-L1 and IDO specific CD4+ T cell clones (FIGS. 32C, 32D, 32H, and 32I)

### Enriched and Newly Detected IDO and PD-L1 T-Cell Clones in Blood and Signs of Tumour Trafficking of Peripherally Expanded Clones Upon Treatment

To track treatment-induced T-cell responses, T-cell receptor (TCR) sequencing of the complementarity-determining region 3 (CDR3) was performed on five patients in peripheral blood (baseline, cycle 3, 6, and 12) and paired biopsies. These 5 patients (MM01, MM02, MM08, MM09, MM13) were selected due to the availability of material and to investigate a balanced patient group with both responders and non-responders. Details on clinical response are shown in FIG. 3A.

Additionally, PBMCs (on treatment) or SKILs were stimulated with the IDO/PD-L1 peptides, then cytokine-producing T-cells were sorted to track vaccine-induced T-cells both in the periphery and at the tumour site.

To identify enriched IDO/PD-L1− specific T-cell clones, TCR rearrangement in sorted IDO/PD-L1 clones, TCR rearrangements in sorted IDO/PD-L1 T cell samples were compared to baseline PBMC samples for each patient. Clonal expansion of vaccine specific TCR rearrangements from samples on vaccination were then tracked using differential abundance framework. Cumulative IDO/PD-L1 T cell frequencies were tracked in post-treatment samples.

No relation between clinical response and the enrichment of vaccine-specific clones was found, but an increase on vaccination of IDO/PD-L1− specific T-cell clones was observed at different time-points in the periphery in all five patients (FIG. 8C).

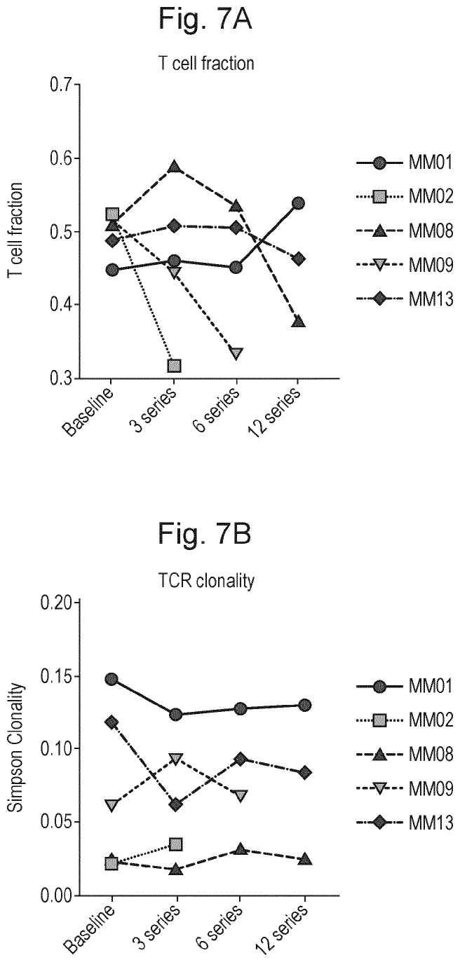

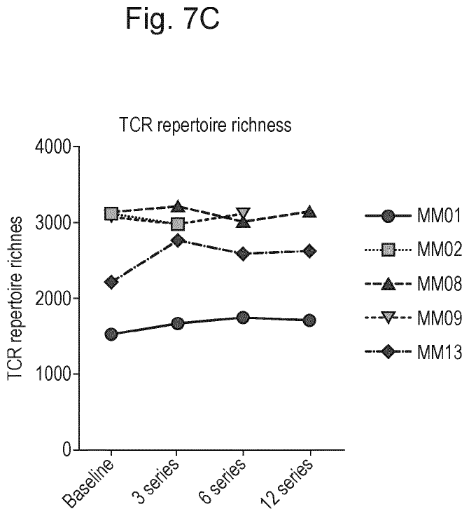

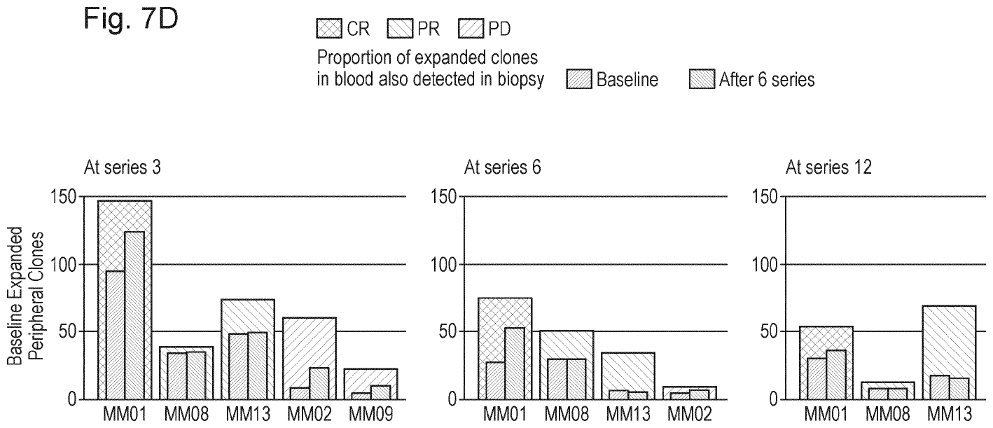

Overall changes in T-cell repertoire in the blood were also investigated. A modest increase in the peripheral T-cell fraction was observed in the three responding patients at cycle 3, while the two non-responding patients had a clear decrease in T-cell fraction. (FIG. 7A) TCR clonality and TCR repertoire richness was then investigated, exploring the proportion of abundant clones and the number of unique rearrangements, respectively. A decreasing peripheral Simpson clonality (FIG. 7B) and increasing TCR repertoire richness (FIG. 7C) was observed in responding patients at cycle 3, which might indicate tumour trafficking upon treatment. Simpson clonality measures how evenly TCR sequences are distributed amongst a set of T-cells where 0 indicate even distribution of frequencies and 1 indicate an asymmetric distribution where a few clones dominate. TCR repertoire richness report the mean number of unique rearrangements. The opposite pattern was seen in non-responding patients. (FIG. 7B and FIG. 7C).

Peripherally expanded clones were tumour-associated and persisted until cycle 12 (latest time-point analysed). The largest peripheral expansion was observed at cycle 3, with the most significant increase observed in patient MM01 (CR). Responding patients had a larger fraction of peripherally expanded clones that were also found in the tumour compared to non-responders. By tracking peripherally expanded clones detected at the tumour site, it was noted that MM01 had a substantial increase after treatment indicating tumour trafficking of peripheral expanded clones. (FIG. 7D).

T-Cell Influx at the Tumour Site with Enriched and Newly Detected IDO and PD-L1 Clones after Therapy

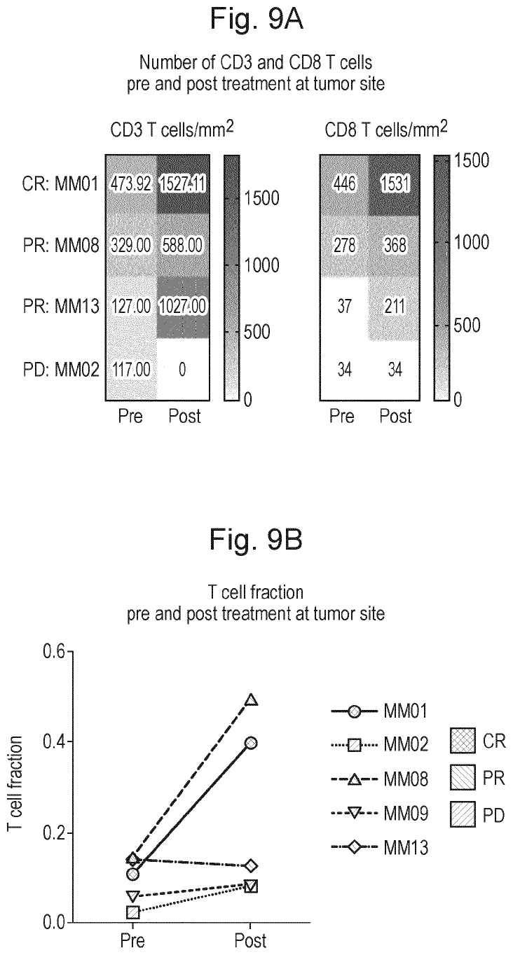

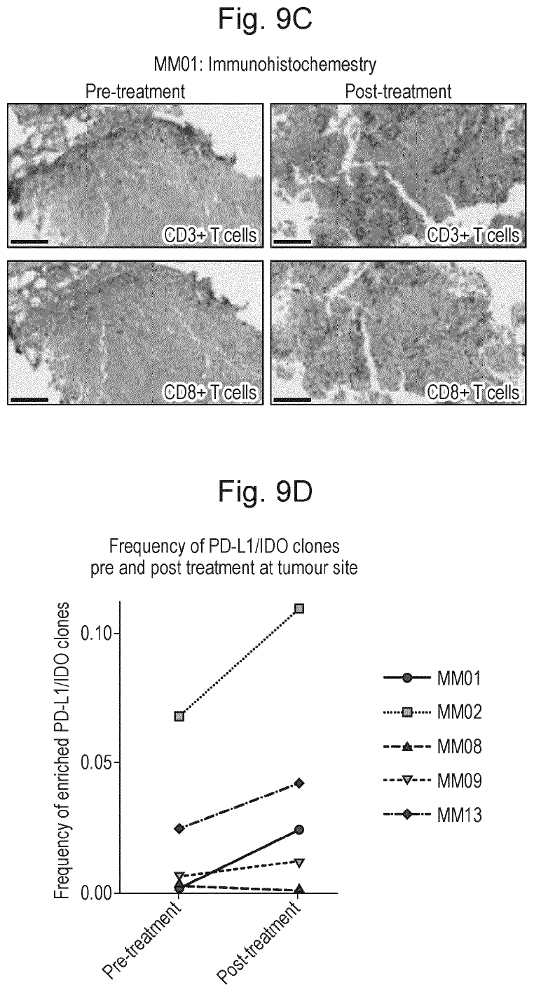

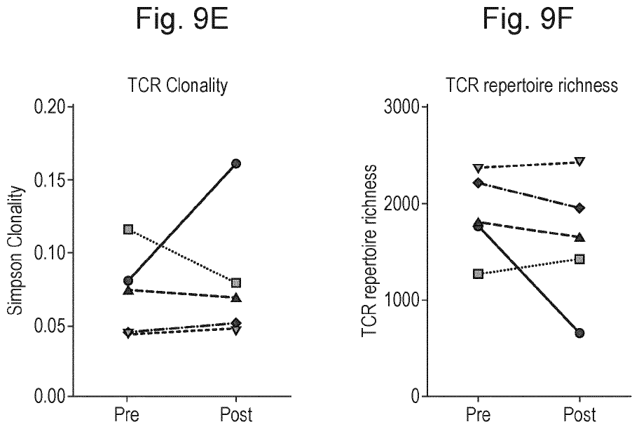

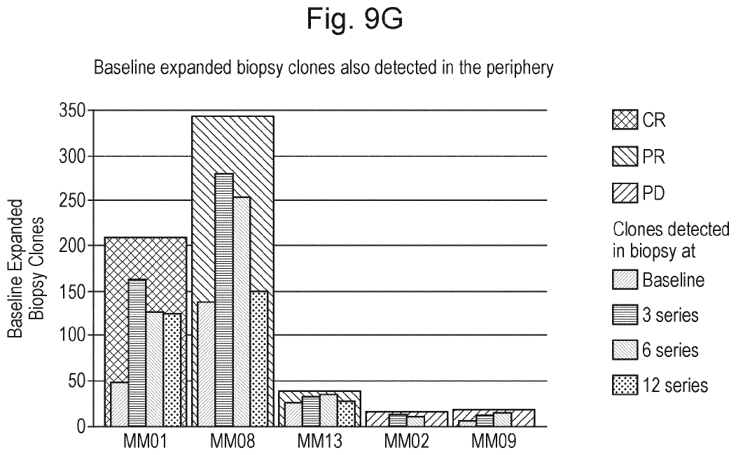

With the observation on increased T-cell fraction and enrichment of IDO and PD-L1 clones in the blood after treatment, the same trends were investigated at the tumour site. Both TCR sequencing and immunohistochemistry (IHC) on paired biopsies from the 5 patients described above showed an increase in T-cell fraction with an influx of CD3 and CD8+ T-cells after treatment in the three responding patients (FIGS. 9A, 9B, and 9C). IHC was not possible on patient MM09 due to tissue loss. The high infiltration of CD8+ T-cells in responding patients suggests tumour antigen recognition by these cells.

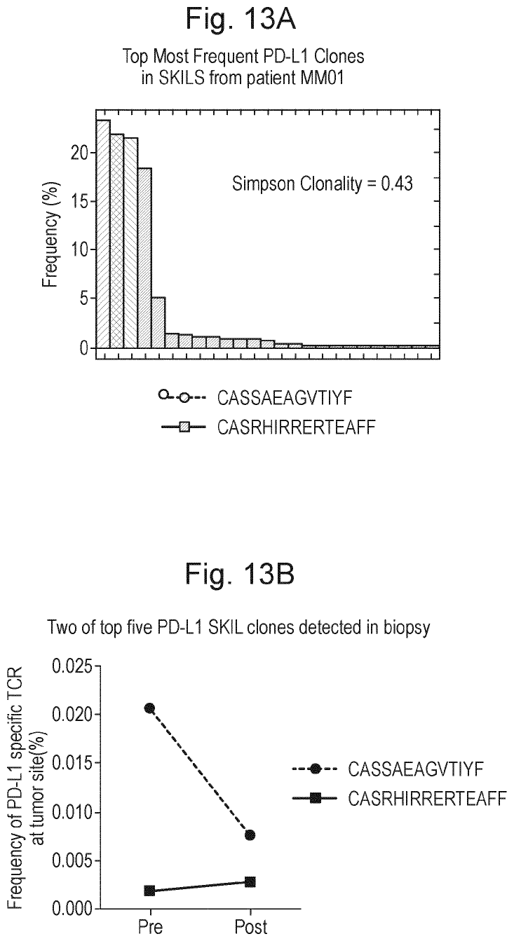

The presence of IDO/PD-L1 vaccine linked T-cells at the tumour site was also investigated. Vaccine associated clones were tracked as combined frequency of IDO and PD-L1 T-cell rearrangements. In biopsies the frequencies at cycle 6 were compared to baseline and showed an increase in vaccine-specific T-cells in four out of five patients, irrespectively of clinical response (FIG. 9D). TCR sequencing of PD-L1 specific SKILs (a more specific culture than the IDO/PD-L1 specific isolations derived from PBMCs on vaccination) and paired biopsies, showed that two of the top five PD-L1 specific SKIL clones were present at the tumour site both before and after treatment. (FIG. 13)

With the focus on the more abundant T-cell clones, overall TCR clonality at the tumour site was investigated before and after treatment. In addition, we explored the number of unique TCR rearrangements, dissecting the lower frequency clones. Patient MM01 had a significant increase in TCR clonality and a decrease in repertoire richness at the tumour site after therapy, indicating a focused tumour repertoire response of selected clones. All three responding patients had a decrease in TCR repertoire richness, which again might indicate a focused tumour response. (FIGS. 9E and 9F)

These data reflects earlier findings which show that MM patients responding to pembrolizumab have a less diverse TCR beta chain repertoire and are more clonal in nature. 19 Deeper analyses showed that the T-cell clones expanded at the tumour site after therapy were also present in the blood at baseline and increased significantly after treatment in 4 out of 5 patients. The highest proportion was detected early at cycle 3. These data again support trafficking of peripherally expanded clones to the tumour site and could indicate that the T-cell response to treatment is derived from pre-existing peripherally tumour associated T-cells. (FIG. 9G)

Treatment-Related Increased T-Cell Function and T-Cell Inflamed TME with Induction of Therapy Targets

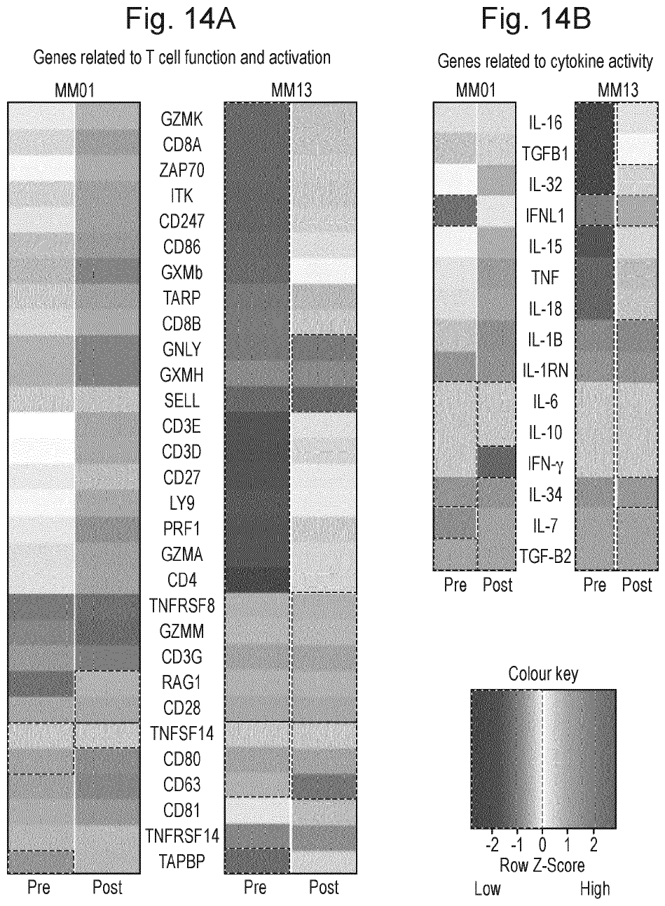

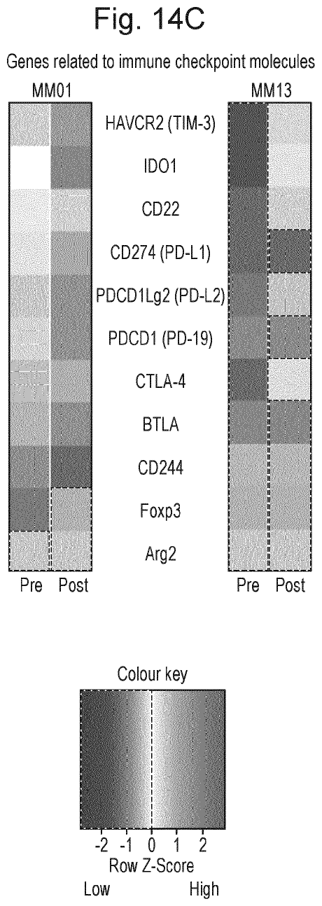

To dissect changes in the TME induced by T-cell influx upon treatment in responding patients, RNA gene expression analyses using the nCounter®PanCancer Immune Profiling Panel from NanoString were performed on paired biopsies from two responding patients (MM01 and MM13). Genes related to adaptive immunity; T-cell activation, effector functions (IFN-γ, TNF-α, IL-15, IL-18) and cytotoxicity were increased in post-treatment biopsies (FIGS. 14A and 14B). Also, genes related to checkpoint inhibitors such as TIM-3, IDO, PD-L1, PD-L2, PD-1, and CTLA-4 increased after treatment, indicating activation of immune cells in the TME. (FIG. 14C)

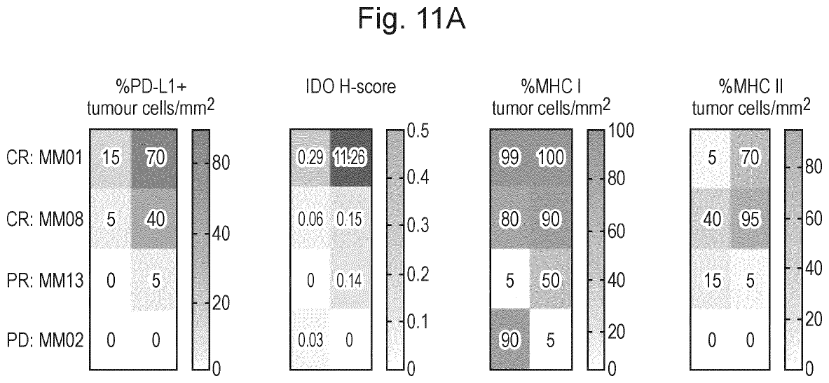

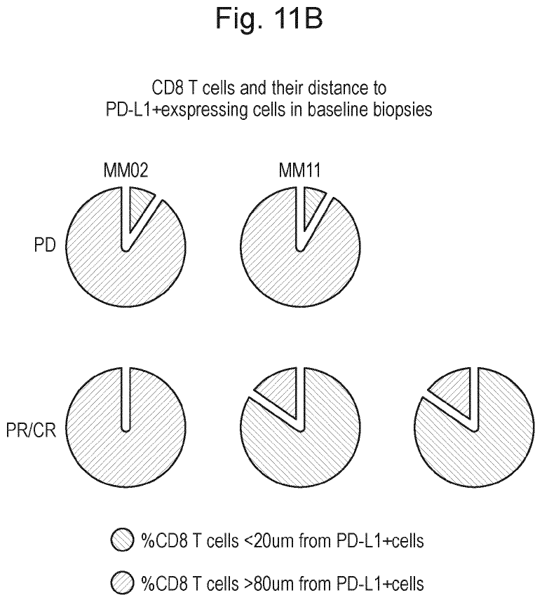

Additionally, IHC on paired biopsies from four patients (MM01, MM02, MM05, MM13) showed an upregulation of PD-L1, IDO, MHCI, and MHCII on tumour cells, indicating a treatment-induced pro-inflammatory response in the three responding patients, except for decrease in MHCII expression in patient MM13. In contrast, the non-responding patient MM02 had a reduction of T-cells present in the tumour after treatment and no expression of PD-L1, IDO, and MHCII, and interestingly total loss of MHCI, indicating tumour immune escape. (FIG. 11A)

CD8+ T-cells and their distance (m) to PD-L1 expressing cells on baseline biopsies in five patients by IHC was investigated. With the exception of patient MM13 (PR), distance and clinical responses were associated: The two responders had reduced distance (<20 m) between cells expressing these markers, compared to non-responding patients (>80 m). This observation indicates that responding patients not only have a higher intratumoral infiltration of CD8+ T-cells, but that these cells can surround and attack PD-L1 expressing immune cells and tumour cells. (FIG. 11B)

### Discussion

In this clinical trial MM1636, 30 patients with metastatic melanoma were treated with a first-in-class immunomodulatory IDO/PD-L1 targeting peptide vaccine combined with nivolumab. The treatment led to an unprecedented high ORR of 80%, with a majority of 43% reaching CR and a striking mPFS of 26 months (95% CI: 15.4-69) was reached. The vaccine represents a novel treatment strategy to activate specific T-cells that target intratumoral regulatory cells (including tumour cells), positively modulating the TME with the induction of local inflammation. These phenomena may further induce checkpoint molecules and rewire the TME towards an increasingly αPD1-permissive state.

The rate of investigator assed ORR in the phase III trial CheckMate067 was 43.7% in the nivolumab monotherapy group and 57% in the nivolumab-plus-ipilimumab group. CR was reached in 8.9% and 11.5%, respectively (Larkin, J. et al. N. Engl. J. Med. 373, 23-34 (2015)). Further, the mPFS of 26 months (95% CI: 15,4-69) in the MM1636 trial is more than twice as long as patients treated with nivolumab-plus-ipilimumab in CheckMate067, where a mPFS of 11.5 (95% CI: 8.7-19.3) months was reached.

Patient baseline characteristics were in general comparable to MM patients that have been treated in CheckMate067, although patients in MM1636 were older (mean age 70) and a larger fraction were PD-L1 positive (57%). 18,20,21 Among patients with PD-L1 negative tumours in MM1636 an ORR of 61.5% was still reached, which would be expected to be around 33% in nivolumab monotherapy first line (Robert, C., N. Engl. J. Med. 372, 320-330 (2015).

To address potential trial bias and the non-randomized set-up, patients in MM1636 were matched on age, PS, Gender, M-stage, LDH-level, PD-L1 status and BRAF-status with a historical control group from the Danish Metastatic Melanoma Database, DAMMED, who were treated contemporarily (2015-2019) with αPD1 monotherapy as standard of care. A significantly higher ORR and CRR was observed in MM1636 compared to matched patients, who had an ORR of 43% and a CRR of 13%, comparable to patients treated in CheckMate067. Restrictions of the synthetic control group is of course that it is partially historic and patient selection outside matching criteria cannot be ruled out (Khozin, S. et al., J. Natl. Cancer Inst. 109, 1359-1360 (2017)).

Numerous contemporary clinical trials are exploring the combination of αPD1 with other immunomodulating agents in advanced melanoma. Talimogene laherparepvec (T-VEC), an oncolytic virus, is approved by the FDA and EMA to treat advanced melanoma. A small phase 1b trial with 21 patients (Masterkey-265) combined T-VEC and pembrolizumab in patients with advanced unresectable melanoma and reached an ORR of 62% and a CR of 33%. Seventy-one percent of the patients in this trial had an M-stage below M1c, this was 40% in our trial. Furthermore, mainly patients with an M-stage below M1c responded to treatment, which was not the case in MM1636.

Epacadostat, an IDO inhibitor in combination with pembrolizumab was tested in a non-randomized phase II trial in 40 αPD1 treatment-naive MM patients with promising results reaching an ORR of 62%. Unfortunately, the phase III trial showed no indication that epacadostat provided improvement of PFS and OS (Long, G. V. et al. Lancet Oncol. 20, 1083-1097 (2019)). Limitations of the phase III trial was the little information on pharmacodynamics, as well as biomarker evaluation to improve the design. The IDO/PD-L1 vaccine differentiates from epacadostat as it is not an IDO inhibitor but targets IDO and PD-L1 expressing cells. Similar vaccines administered as monotherapy induced objective responses in lung cancer and basal cell carcinoma, while epacadostat as monotherapy in 52 patients showed zero responses (Iversen, T. Z. et al. Clin. Cancer Res. 20, 221-232 (2014); Kjeldsen, J. W. et al., Front. Immunol. 9, 1-6 (2018)).

The overall safety and tolerability findings are comparable to αPD1 monotherapy. Injection site reactions were exclusive to the vaccine. However, these side effects were transient and mild in most patients and most likely due to the adjuvant montanide.

Numerous vaccines induced changes in the blood and at the tumour site were observed. Peripheral IDO and/or PD-L1 specific T-cells were detected in vitro in over 93% of the patients on vaccination unrelated to patient HLA-type. The immune responses were persistent in patients who surpassed follow-up at data cut-off and were still detectable up to 6 months after the last vaccine, suggesting induction of memory T-cells. Despite most patients showing an immune response to the vaccine in the blood, we observed no correlation between vaccine-induced responses in blood and clinical responses. TCR sequencing on five patients confirmed enrichment of IDO/PD-L1 T-cell clones in the blood at different time points after treatment. Furthermore, an increase of enriched IDO/PD-L1 clones was observed in 4 out of 5 patients at the tumour site after treatment irrespectively of clinical response.

Phenotypic characterization showed that vaccine-specific T-cells, which were expanded in vitro with IL-2 from the blood of vaccinated individuals, were both CD4+ T-cells and CD8+ T-cells. Vaccine-specific T-cells expressed CD107a and produced IFN-γ and TNF-α upon stimulation with the cognate target, indicating their cytolytic capacity. Future studies will validate if the pro-inflammatory profile and cytolytic features observed in vaccine specific T cells in vitro are also seen ex vivo.

Despite a limited number of paired biopsies, due to either patient decline or the fact that a large fraction of responding patients had no assessable tumours after 6 cycles, we observed trends indicating treatment-induced general T-cell influx in responding patients. It has been shown that proliferation of CD8+ T-cells in the tumour after αPD1 treatment is associated with radiographic reduction in tumour size (Tumeh, P. C. et al. Nature 515, 568-571 (2014)). Additionally, a large proportion of expanded peripheral TCR clones were tumour-associated and the most considerable amount of clonal expansion was observed early at cycle 3. For the CR patient included in the TCR 392 sequencing analyses (MM01) the number of peripheral expanded clones present at the tumour-site increased after treatment compared to baseline, indicating tumour-trafficking of peripheral expanded clones.

Gene expression analyses (2 paired biopsies) and IHC (5 paired biopsies) further demonstrated that the treatment induced a pro-inflammatory TME in responding patients with signs of T-cell activation and cytotoxicity, and increased cytokine activity. This may lead to further upregulation of IDO, PD-L1, MHC I and MHC II on tumour cells leading to more treatment targets. It has been shown that, following vaccination with a cancer vaccine, PD-L1 expression is increased on tumour cells due to recruitment of tumour specific T-cells and upregulation of adaptive immune resistance pathways in the TME (Wang, T. et al. Nat. Commun. 9, 1-12 (2018)).

Treatment with nivolumab monotherapy enhances PD-L1 expression and it is therefore problematic to discriminate the effect of the vaccine as compared to nivolumab (Vilain, R. E. et al. Clin. Cancer Res. 23, 5024-5033 (2017)). In conclusion, here we report an impressive response rate, complete response rate, and mPFS for a first-in-class immune modulating vaccine combined with nivolumab. This may be a first step toward a new treatment strategy for patients with metastatic melanoma. Limitations are the low number of patients treated at a single institution, and the lack of a randomized design with αPD1 monotherapy as comparator. Studies in αPD1 resistant or refractory melanoma are ongoing as well as biomarker analysis for selecting patients at a higher likelihood to benefit from the combination vs αPD1 monotherapy. A larger randomized trial will validate these findings and determine the specific contribution of the vaccine to clinical responses and changes in the TME. In December 2020, the Food and Drug Administration (FDA) granted breakthrough therapy designation for the 10102/10103 vaccine combined with αPD1 in metastatic melanoma based on data from the MM1636 trial.

### Example 4—Identification of Immune Profiles Correlative of Clinical Response in the MM1636 Trial

### Patient Population, Treatment Plan and Samples

Study design, eligibility criteria, and treatment plan of the clinical trial are described above. Briefly, patients were treated with an IDO/PD-L1 vaccine every second week for the first 6 administrations and thereafter every fourth week up to 15 vaccinations in total. Nivolumab was administered biweekly for up to two years until progression or complete response. This was a non-randomized, single-center phase I/II study at the Department of Oncology at Herlev and Gentofte University hospital, Denmark. ClinicalTrials.gov, identifier: NCT03047928. All patients provided written informed consent.

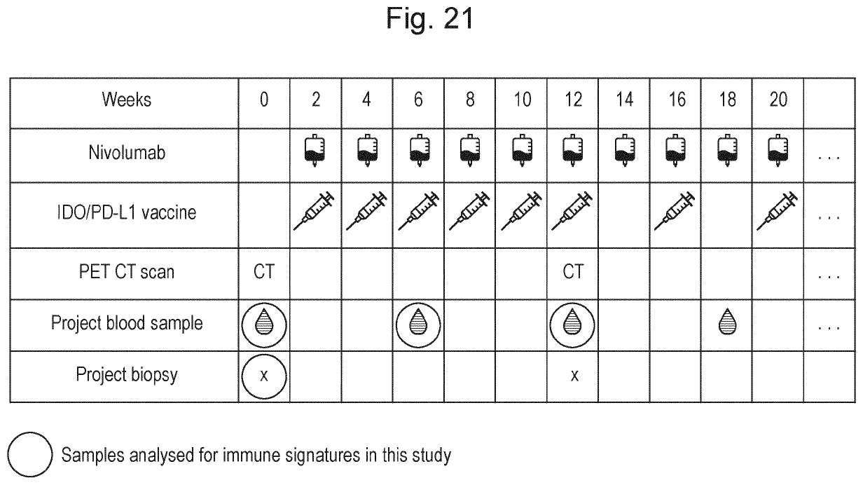

Baseline biopsies were taken one-week prior to treatment start and after 6 cycles of treatment. Baseline blood samples were taken the same day as the first treatment (before), at cycle 3, cycle 6 and thereafter at every evaluation scan every third month. In this study we looked into baseline biopsies and peripheral blood at baseline, at cycle 3 and cycle 6 (see FIG. 21).

### Collection of Blood and Biopsies and Isolation of PBMCs and Serum

Peripheral blood was drawn from all patients at baseline, cycle 3 and 6 in heparinized tubes. After a maximum of 4 hours PBMCs were separated using Lymphoprep (Medinor) density gradient and cryopreserved in 90% human AB serum (Sigma.Aldrich, Ref. No H4522-100 ml) and 10% DMSO using controlled-rate freezing (Cool-Cell, Biocision) in −80° C. The next day they were moved to −140° C. freezer until used for analysis. Serum samples were collected at baseline, cycle 3 and 6. Serum tubes were spun at 3000 ref for 10 minutes after a maximum of 4 hours and transferred immediately to a −80° C. freezer and stored until further processing.

Baseline biopsies were taken with a 1.2 mm needle when assessable on tumour metastasis and afterwards formalin fixed, paraffin-embedded (FFPE).

### Immunohistochemistry (IHC)

IHC staining was performed at HalioDX Service Laboratory. In short, FFPE blocks were stained for CD3+, CD8+, MHC I (on tumour cells), MHC II (on tumour cells), IDO (on tumour and immune cells) and PD-L1 (on tumour cells) as described above.

### RNA Gene Analysis (NanoString)

RNA expression profiling was performed by HalioDX using NanoString nCounter Analysis System on 776 targeted genes as described above.

### Phenotyping of PBMCs Using Multi-Color Flowcytometry

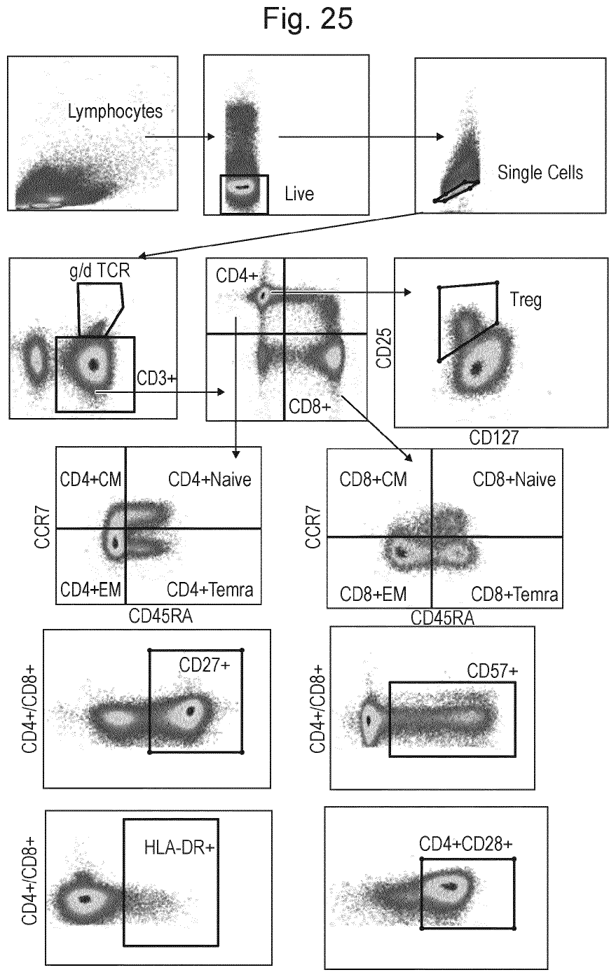

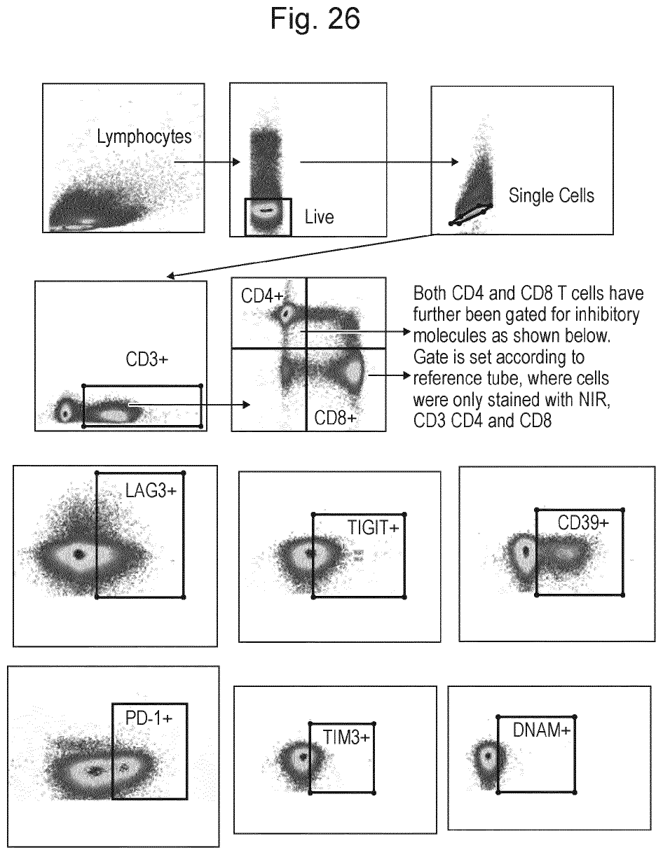

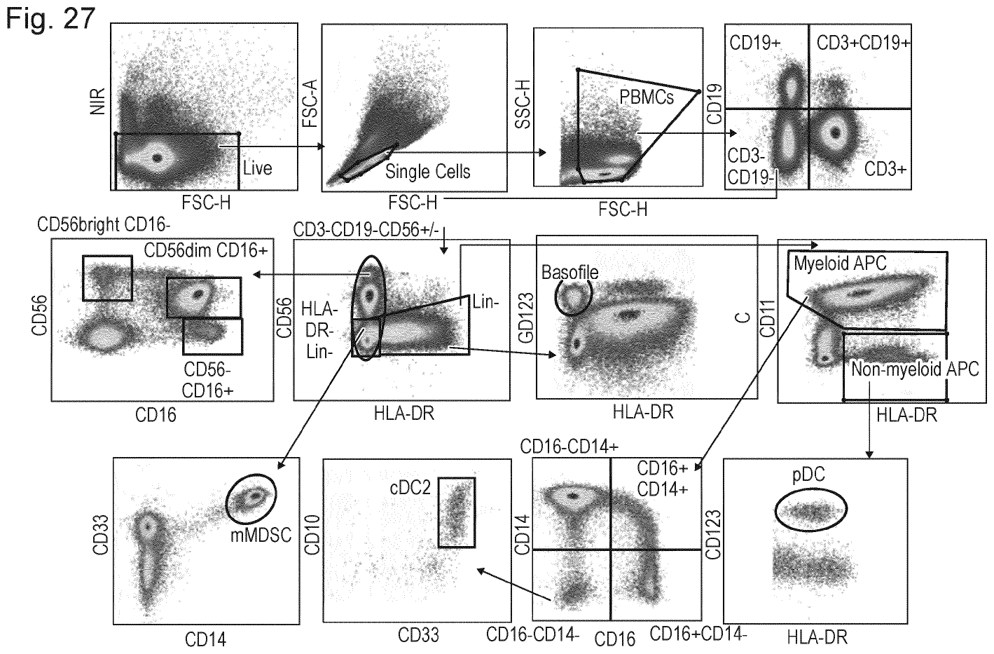

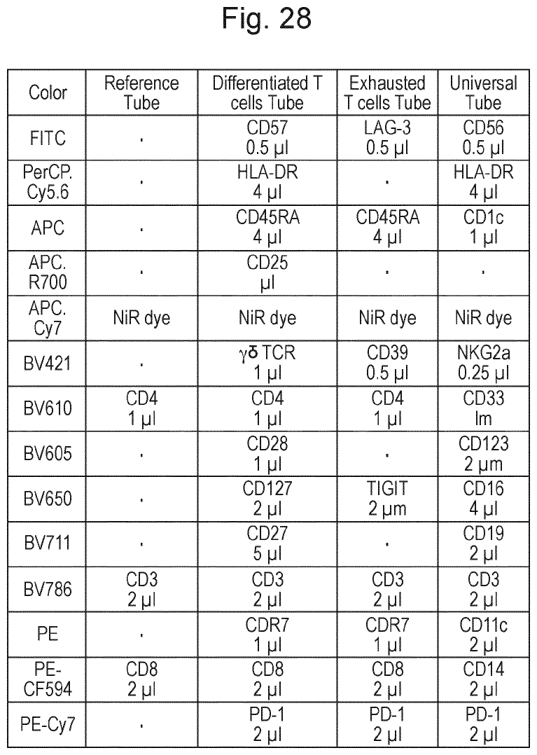

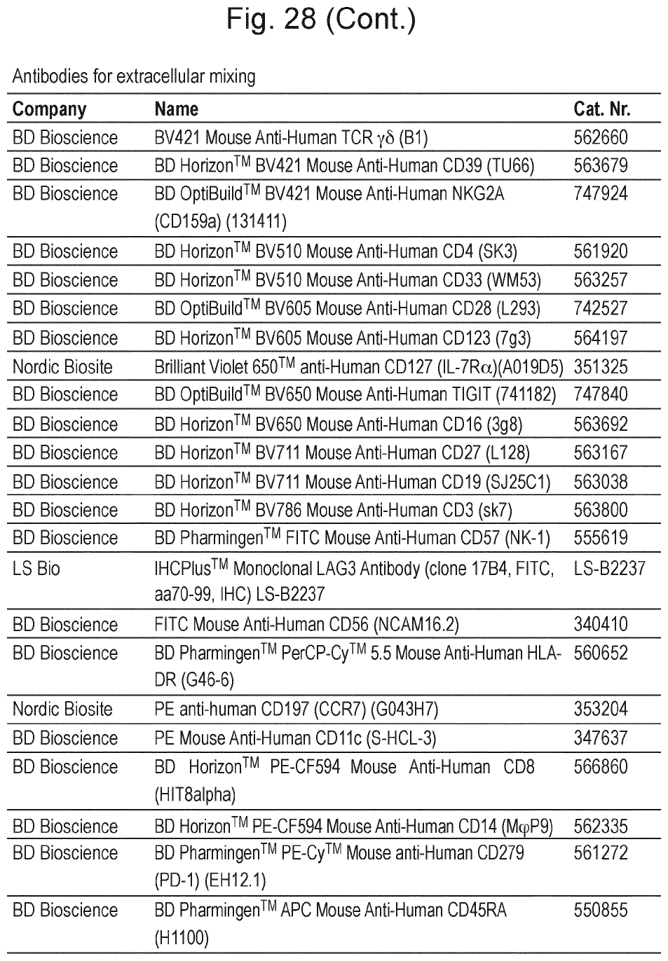

For surface staining of PBMCs, fluorochrome-labeled anti-mouse antibodies from either BD Biosciences or Biolegend was used. Extracellular antibody mixtures containing 0.25-4 μl/well of each antibody, 10% Brilliant Violet Stain Buffer (BVSB)-plus (10×) (BD biosciences, Cat. No. 566385) and phosphate-buffered saline (PBS) was made. Live/Dead Fixable Near-IR (NIR) Dead Cell stain kit was obtained from ThermoFischer and was diluted 1:100 in EDTA Buffer (Life Technologies, Cat. No. 15575-038). PBMCs were thawed, washed with PBS, stained with NIR and incubated in the dark at 4° C. Relevant antibodies were added, and the samples incubated in the dark at 4° C. After incubation the cells were washed, resuspended in PBS and placed on ice until acquisition. Flow cytometry analysis was conducted using a Novocyte Quanteon (ACEA Biosciences) and analyzed using FlowJo Software. Gating strategy is shown in FIG. 25-27. Table of antibodies used is shown in FIG. 28.

Data visualization and statistical analyses were done in GraphPad Prism (version 8.0.2). Wilcoxon matched pairs signed rank t test was used to test the level of significance in paired observations. A Mann-Whitney U test was used to compare ranks of unpaired observations.

### Assessment of Kynurenine (Kyn)/Tryptophan (Trp) Ratio Inpatient Serum

Twenty essential amino acids, including kynurenine (kyn) and tryptophan (trp), concentration in plasma samples was measured using liquid chromatography-tandem mass spectrometry (LC-MS/MS) as described elsewhere in all patients at baseline and at cycle 3 and 6 (Bornø, A. & Van Hall, G. J. Chromatogr. B Anal. Technol. Biomed. Life Sci. 951-952, 69-77 (2014)).

### Results

### Baseline Tumour Immune Profiles

Thirty anti-PD1 treatment naive patients with histologically confirmed metastatic melanoma (MM) were included in the MM1636 trial. Patient characteristics are described above; briefly mean age was 70 years, 60% were M1c, 35% had elevated LDH, 43% were PD-L1 negative (<1%) and 38% were BRAF mutated.

The combination treatment resulted in notable clinical efficacy and prolonged progression-free survival (PFS). An overall response rate of 80% was reached and a median PFS of 26 months. Thus, 20% of the patients did not respond and 30% had a PFS below 9 months. Immunohistochemistry (IHC) analysis for PD-L1 expression was performed as standard melanoma recurrence diagnostics. 94% of the patients with PD-L1 expression >1% (based on the tumor proportion score as measured by PD-L1 expression by IHC) responded to the treatment, while 61% with <1% PD-L1 expression responded.

To search for potential biomarkers that could predict response, baseline immune profiles were examined at the tumour site. These immune analyses were performed on voluntary trial specific tumour biopsies and therefore not available on all patients due to e.g. patient decline and lack of assessable tumour lesions. Thus, the following analysis were performed on tumour biopsies from eight patients representing both responding and non-responding patients.

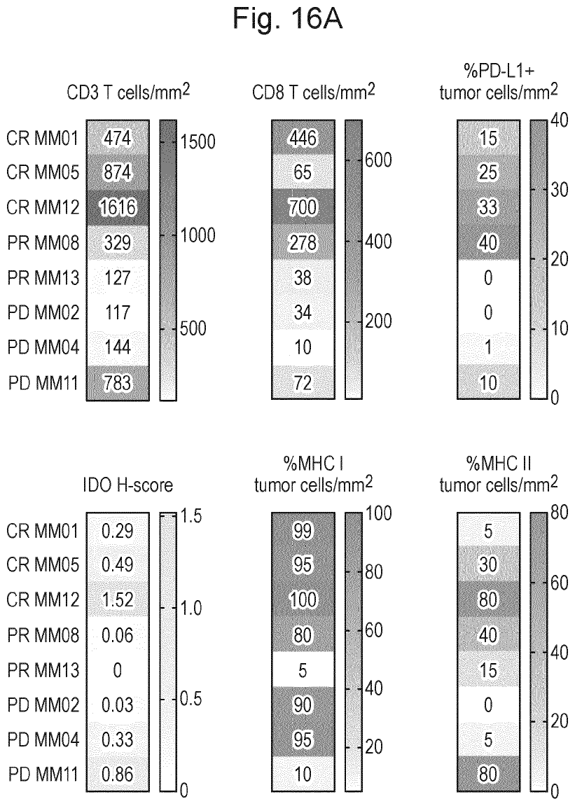

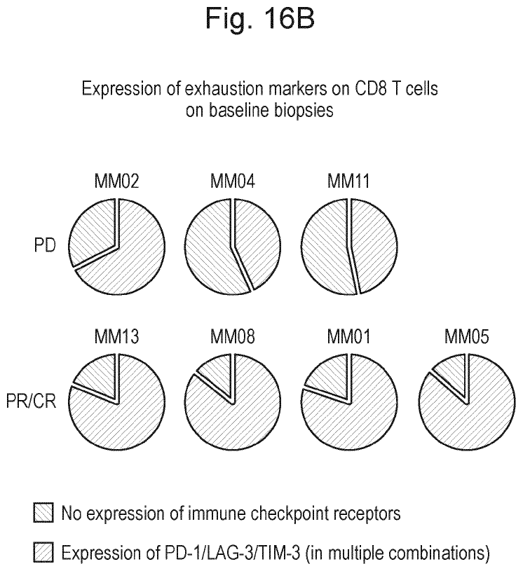

Baseline T-cell infiltration in the tumour was evaluated by IHC with staining of CD3 and CD8 expression performed on 8 patients (MM01, MM02, MM04, MM05, MM08, MM11, MM12, MM13), reflecting a balanced cohort with both responding (3 CR and 2 PR) and non-responding patients (3 PD). Four of the five responding patients had a high T-cell infiltration, while two of the three non-responding patients had almost no T-cells present in their tumour (FIG. 16A).

Baseline expression of checkpoint inhibitor molecules (PD1, LAG3 and TIM3) were also analyzed by IHC. More than 50% of the CD3+CD8+ positive T-cells expressed PD-1, LAG-3 or TIM-3 alone or in combination in all patients except for patient MM04 and MMII (both non-responders) who expressed around 40% (FIG. 16B). This suggests that responding patients might have more activated T-cells which upregulate checkpoint inhibitory molecules upon antigen stimulation.

Further, as demonstrated in FIG. 16A, four out of five responding patients analysed tended to exhibit higher expression of MHC-II on tumour cells compared to the three non-responders where only one out of three had high MHC-II expression. On the other hand, MHC class I was highly expressed in most patients and no association with clinical response was found. Similarly, no association was seen between IDO expression and clinical responses (FIG. 16A).

To conclude, IHC analysis of baseline tumour samples showed that responders tended to have a more “hot” tumour, e.g. high T-cell infiltration and increased expression of MHC-II and PD-L1 on tumour cells.

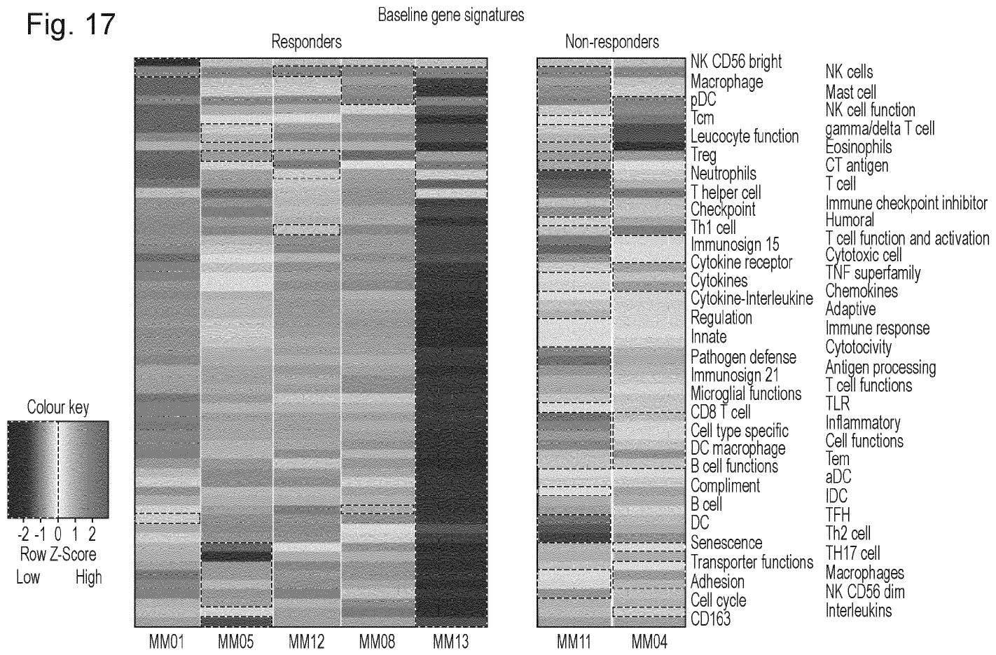

Additionally, RNA gene expression analyses of 776 genes related to innate and adaptive immunity was performed on baseline biopsies in 7 patients (same patients that were analysed with IHC, except patient MM12 due to tissue loss) using NanoString technology. Responding patients tended toward higher T-cell activation at baseline, which does not correlate directly to the levels of CD3 and CD8 T-cell infiltration (FIG. 17). The one outlier was patient MM11, who had a high T-cell infiltration but low expression of genes related to T-cell activation. Notably, the tumour cells in this patient lost MHC I expression during treatment.

All in all, gene expression analyses showed that responding patients tended to have a higher expression of innate and adaptive immune profiles (indicative of a “hot tumour”) compared to non-responders, except for patient MM13 who stands out as a responding patient with a cold baseline tumour (FIG. 17).

### Baseline Peripheral Blood Immune Cell Profiles

A multicolour flow cytometry panel was used to compare immune profiles in blood between responders and non-responders (FIG. 28). Immune phenotyping was performed on PBMCs and analyzed at baseline on all 30 patients.

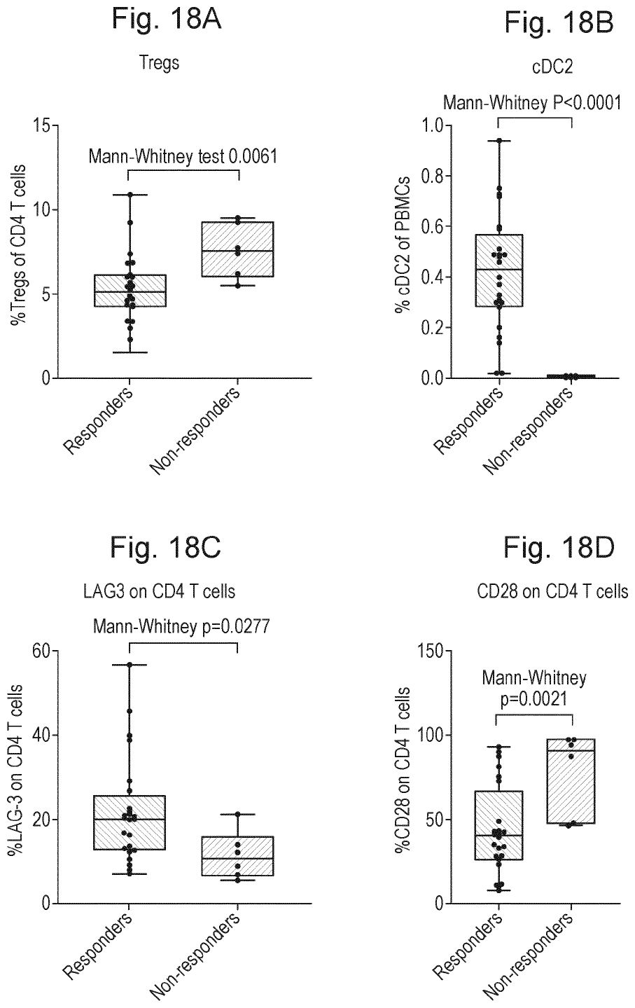

At baseline, significant differences were observed between responding and non-responding patients. Responding patients had a significantly lower percentage of Tregs (CD4+CD25^{high}CD127^{low}) (5.2% vs 7.5% p=0.01) (FIG. 18A). A significant decrease in expression of CD28 on CD4+ T-cells in responders was observed as compared to non-responders (43% vs 78% p=0.0065). Further, a significantly higher percentage of CD4+ T-cells expressed LAG-3 in responders as compared to non-responders (20.1% vs 10.56% p=0.0277) (FIG. 18C and FIG. 18D).

Responding patients also had a significant higher percentage of classical dendritic cells (cDC2) (CD3-CD19-CD56-CD11c+CD16-CD14-CD33+CD1c+) as a percentage of PBMCs compared to non-responders (0.42% vs 0.0005% p=0.0002) (FIG. 18B).

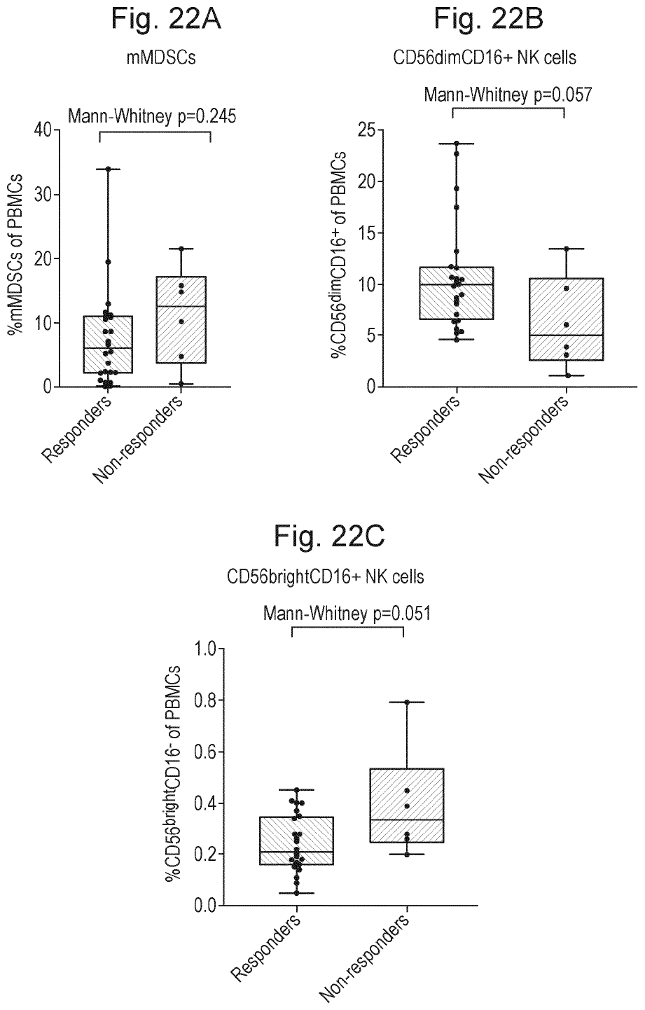

Finally, a trend towards a lower percentage of monocytic-myeloid derived suppressor cells (mMDSCs) (CD3-CD19-CD56-HLADR-CD14+CD33+) as a percentage of PBMCs was observed in responding patients compared to non-responders (6% vs 12.5% p=0.24) (FIG. 22A). There was also a non-significant trend towards a higher percentage of CD56^{dim}CD16+NK cells as percentage of PBMCs and a lower percentage of CD56^{bight}CD16− NK cells as percentage of PBMCs compared to non-responders (FIGS. 22B and 22C).

No difference in baseline fractions of CD4 or CD8 T-cells and their differentiated subtypes into naïve, effector memory, central memory and effector memory RA (based on CD45RA and CCR7 expression) were found in responding and non-responding patients. Also, expression of PD-1, CD27, CD57, CD39, TIGIT, TIM3 and HLA-DR on both CD4 and CD8 T-cells were evenly distributed between the two groups. Finally, no difference in the distribution of B-cells, 76 T-cells, plasmacytoid dendritic cells (pDCs), classical and non-classical monocytes were observed at baseline between responding and non-responding patients (data not shown).

### Early On-Treatment Immune Cell Changes

Multicolor flowcytometry was performed on PBMCs at treatment cycle 3 (week 6) and cycle 6 (week 12) in all patients to further investigate immune cell subtypes of potential relevance for treatment outcome. In the responding group (n=24) PBMCs from all three time points were available. In the non-responding group (n=6) material from series 6 was missing on three patients due to rapid progression.

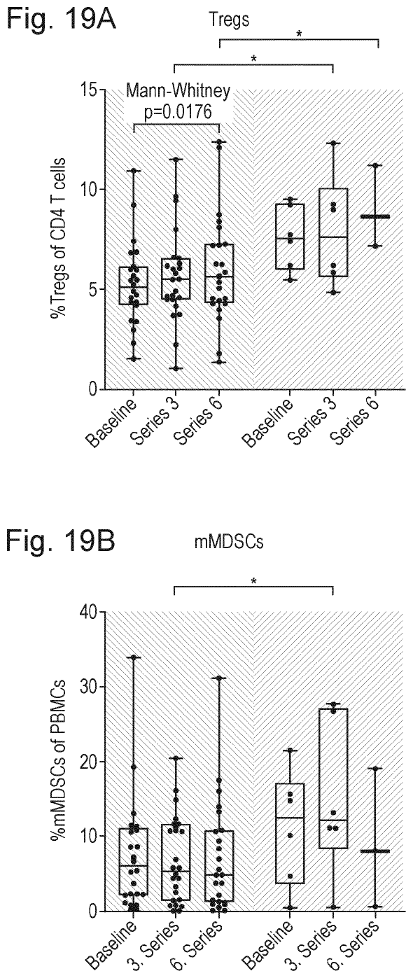

Non-responding patients had a higher percentage of immunosuppressive cells (Tregs and mMDSC) in their blood at baseline compared to responding patients. The same pattern was observed at cycle 3 and cycle 6, where non-responding patients still had a higher percentage of both Tregs and mMDSC. Nevertheless, a significant increase in the percentage of Tregs was observed in responding patients from baseline to cycle 6. The same trend was observed in non-responding patients but did not reach statistical significance (FIG. 19).

Overall, the significant differences in cell populations and surface marker expression observed at baseline between responders and non-responders (mMDSCs, Tregs, cDC2, and expression of LAG3 and CD28 on CD4 T cells) were also seen after treatment (data not shown).

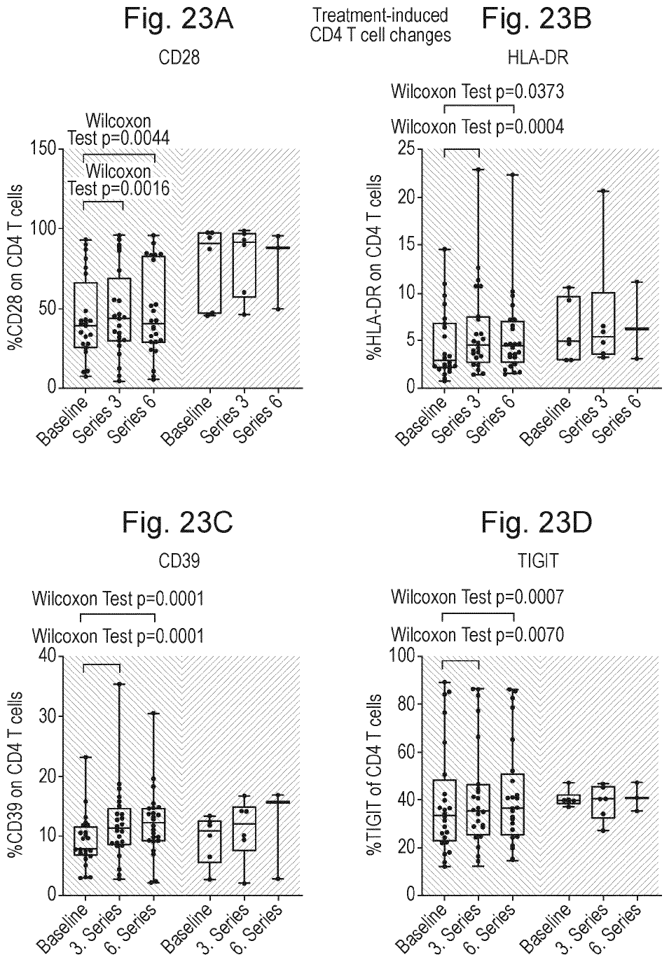

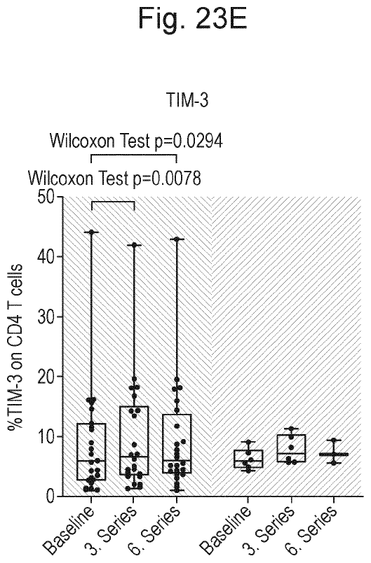

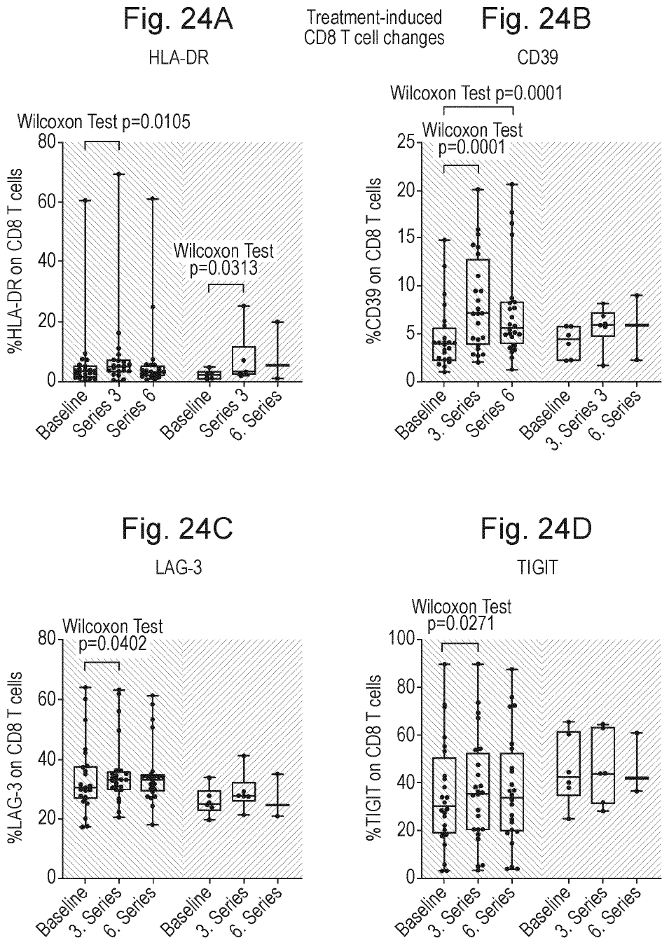

A significant increase of the activation/exhaustion markers such as CD28, HLA-DR, CD39, TIGIT, and TIM-3 was observed on CD4 T-cells after treatment, while a significant increase of HLA-DR, CD39, LAG-3, and TIGIT was observed on CD8 T-cells in responding patients indicating a more general immune activation after treatment. The same tendency was observed in non-responding patients (FIG. 23 and FIG. 24).

The expression of the inhibitory marker NKG2a also increased significantly on both CD56^{dim} and CD56^{bright} NK cells which could indicate an activation of both NK cell subtypes on treatment. Again, the same tendency was observed in non-responding patients. (data not shown).

### Kynurenine/Tryptophan Ratio as a Potential Early On-Treatment Biomarker of Response

The kynurenine (kyn)/tryptophan (trp) ratio has been suggested to mirror IDO activity (Uyttenhove, C. (2003) Nat. Med. 9, 1269-1274). An increase in the kyn/trp ratio is generally indicative of systemic immune modulation and has been implicated in the progression of different cancer types.

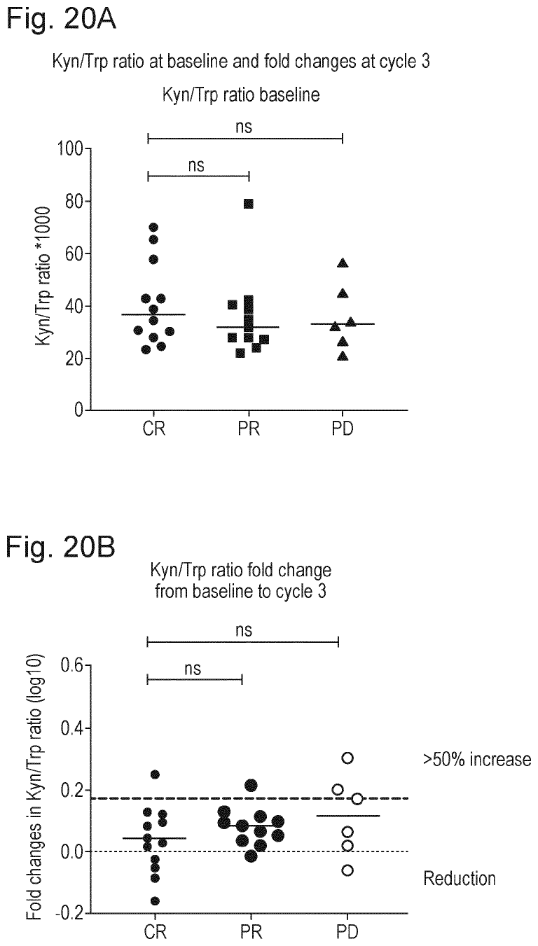

23 measurements of Kyn and Trp levels from sera at baseline and cycle 3 were performed on all 30 patients. No significant differences between baseline levels of Kyn/Trp ratio in CR, PR or PD patients were observed (FIG. 20A). Fold changes in Kyn/Trp ratio from baseline to cycle 3 were compared between CR, PR and PD patients. A tendency towards a higher increase was observed in the non-responding patients, indicating an adaptive resistance mechanism in these patients. The tendency was not significant which might be due to the limited number of progressing patients (FIG. 20B).

### Discussion

Numerous studies have explored predictive biomarkers for response in melanoma patients treated with immune checkpoint inhibitors, such as anti-PD1 (nivolumab and pembrolizumab) and anti-CTLA-4 (ipilimumab) (Subrahmanyam, P. B. et al., J Immunother Cancer. 2018 Mar. 6; 6(1):18.24; 20; Nebhan & Johnson Expert Review of Anticancer Therapy vol. 20 137-145 (2020)). To date, most biomarkers have been identified at the tumour site, but biomarkers in peripheral blood would naturally be of great interest, due to the ease of accessing blood versus tumour tissue. Another advantage of finding biomarkers in blood is the homogeneity compared to tumours, where a biopsy in one metastasis can be very different from another metastasis or even the same (Bedard, P. L. et al., Nature (2013) 501(7467): 355-64). Baseline and early on-treatment immune profiles correlative of response at the tumour site and in peripheral blood were investigated in patients treated with an immune modulatory vaccine comprising IDO and PD-L1 peptides in combination with nivolumab in a phase I/II clinical trial. Despite the fact that most patients responded to the treatment, 20% did not benefit, and 30% had a PFS below 9 months. Therefore, the aim was to clarify possible differences in immune profiles between responders and non-responders, to better understand why some patients respond to the treatment while others do not and to help improve patient selection and optimize the design of future clinical trials with this combination treatment.

Available material was evaluated for immune profiles at the tumour site correlative of response. Due to the limited number of baseline biopsies we could not apply statistics, but trends were observed that responding patients had high T-cell infiltration as well as high expression of PD-L1, while non-responding patients had low T-cell infiltration and almost no PD-L1 expression.

High T-cell infiltration at the tumour site and high expression of PD-L1 on the tumour cells are two factors known to correlate to response but are not optimal as biomarkers, since a fraction of PD-L1 negative and low T-cell infiltrating tumours still respond to anti-PD1 therapy (Fusi, A. et al. Lancet Oncol. 16, 1285-1287 (2015); Tumeh, P. C. et al. Nature 515, 568-571 (2014). The same trend was found in the present study where one patient (MM13) with a long-term partial response had a cold tumour at baseline with low T-cell infiltration and no PD-L1 expression, while a non-responding patient (MM11) had high T-cell infiltration and high PD-L1 expression. This again highlights the potential difficulties associated with the use of these biomarkers.

Expression of the T cell exhaustion markers PD-1, TIM-3, and LAG-3 (in multiple combinations) were higher in the four responding patients compared to the three non-responders, indicating that these T-cells have seen tumour antigen, and might be more responsive to treatment strategies targeting these immune checkpoint molecules.

The fact that patient MM13 has signs of a cold tumour, but still responds to treatment could be explained by the fact that the few T-cells that are present at the tumour have been activated (high expression of checkpoint inhibitors on T-cells) and indicating cytotoxic capacities, which might have been enough to achieve tumour control. On the other hand, a non-responding patient (MM11) had signs of a hot tumour with high T-cell infiltration and 10% PD-L1 expression, but absolutely no expression of MHC I on tumour cells, indicating tumour immune escape.

A correlation between a high expression of IDO on tumour cells and response to anti-CTLA4 therapy has been reported, but no association has been found with response to anti-PD1 therapy (Hamid, O. et al. J. Transl. Med. 9, 204 (2011). In the present study, no association between IDO expression on tumour cells and clinical response was observed.

MHC II expression on tumour cells, but not MHC I, has been shown to correlate with clinical response, PFS, and OS, as well as CD4 and CD8 T-cell infiltration in melanoma patients treated with anti-PD1 (Johnson, D. B. et al. Nat. Commun. 7, (2016)). The same tendency was observed in the present study. However, the present data indicate that MHC II is not a suitable predictive biomarker due to a large overlap in the MHC II expression between the responders and non-responders.

Baseline blood samples were analysed for several immune cell populations by flow cytometry. Interestingly, it was observed that responding patients had significantly lower percentage of Tregs at baseline compared to non-responders and non-responding patients had a trend towards higher proportion of mMDSCs as a percentage of PBMCs at baseline. At treatment cycle 3, the percentage of Tregs and mMDSCs were both significantly higher in the non-responder group compared to the responding group.

DCs are a group of specialized antigen-presenting cells. At baseline, a significantly higher percentage of cDC2 was observed in responding patients, but no difference was seen for pDCs. Classical DC2 are known to prime CD4 T-cells for anti-tumour function and they might play an important role for patients receiving treatment with a combination of an IDO/PD-L1 immune modulatory vaccine and anti-PD1 therapy.

No baseline differences in the percentage of CD4 and CD8 T-cells or their differentiation into naïve, CM, EM or TemRA, but expression of inhibitory as well as co-stimulatory molecules were of interest. CD28 is a co-receptor expressed on both CD4 and CD8 T-cells and provide co-stimulatory signals required for T-cell activation and survival, but it has also been shown that CD4 T-cells that lack expression of CD28 (CD4+CD28−) can be classified into cytotoxic T-helper type-1 cells known to produce IFN-7, IL2, perforin and granzyme (Maly, K. & Schirmer, M. J. Immunol. Res. (2015)).

It was demonstrated that responding patients had lower expression of CD28 on CD4 T-cells compared to non-responders, which could indicate the importance of CD4 T-cells in response to this treatment. Furthermore, a significant higher expression of LAG-3 on CD4 T-cells was found in responding patients compared to non-responders. LAG-3 is a checkpoint molecule which mainly binds to MHC-II molecules and provides an inhibitory signal to the T-cell, and a higher expression of the marker might indicate a higher exposure to tumour antigens in these patients.

Early on-treatment immune profiles in blood at cycle 3 and 6 were evaluated in order to identify immune profiles predictive of response to treatment with IDO/PD-L1 immune modulatory vaccine in combination with anti-PD1 therapy. Overall, the baseline differences found between responders and non-responders remained significant at cycle 3 and 6. No new immune subsets that were significantly different between the two groups were observed. However, changes revealing immune activation with higher expression of activation/inhibitory markers after treatment of both CD4 and CD8 T-cells compared to baseline were observed. This observation was significant in the responding group and the same tendency was observed in the non-responding group.

It has been described that the Kyn/Trp ratio increases as an adaptive resistance mechanism in response to αPD1 therapy. Furthermore, is has been observed that >50% increase in kyn/trp ratio upon αPD1 treatment is strongly associated with poor prognosis. The same tendency was observed in the present study where the median fold change was lower in complete responders compared to both partial responders and patients with progressive disease.

In conclusion, intra-tumoral and peripheral baseline immune parameters correlative of response in patients treated with an IDO/PD-L1 peptide vaccine and anti-PD1 therapy have been identified. High expression of PD-L1 and MHC II on tumour cells, high T-cell infiltration, and higher expression of inhibitory molecules (LAG-3, TIM-3 and PD-1) on CD8 T-cells were prevalent in tumour lesions from responders.

Peripheral blood analyses were conducted on all patients. Here it was demonstrated that responding patients had significantly lower level of Tregs at baseline compared to non-responders and the same tendency was observed for mMDSCs. Early on-treatment changes showed signs of immune activation with higher expression of inhibitory/activation molecules on both CD4 and CD8 T-cells as well as a significant increase in the percentage of Tregs of CD4 T-cells, indicating homeostatic control. This was significant in the responding group, but the same tendency was observed in the non-responding group.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A method for treating a cancer in a subject in need thereof comprising administering to the subject:
a) a first immune checkpoint polypeptide or a polynucleotide encoding the same;
b) a second immune checkpoint polypeptide or a polynucleotide encoding the same; and
c) an immune checkpoint inhibitor.

2. A method of preventing disease progression in a subject suffering from a cancer comprising administering to the subject:
a) a first immune checkpoint polypeptide or a polynucleotide encoding the same;
b) a second immune checkpoint polypeptide or a polynucleotide encoding the same; and
c) an immune checkpoint inhibitor.

3. A method of reducing tumor volume in a subject suffering from a cancer comprising administering to the subject:
a) a first immune checkpoint polypeptide or a polynucleotide encoding the same;
b) a second immune checkpoint polypeptide or a polynucleotide encoding the same; and
c) an immune checkpoint inhibitor.

4. The method of any one of claims 1-3, wherein the subject has not previously received treatment with the immune checkpoint inhibitor.

5. The method of any one of claims 1-3, wherein the subject has previously received treatment with the immune checkpoint inhibitor.

6. The method of claim 5, wherein the subject was refractory to the treatment with the immune checkpoint inhibitor or developed resistance to the immune checkpoint inhibitor during the course of the previous treatment.

7. The method of any one of claims 1-6, wherein the first and second immune checkpoint polypeptide are independently selected from an IDO1 peptide, a PD-1 peptide, a PD-L1 peptide, a PD-L2 peptide, a CTLA4 peptide, a B7-H3 peptide, a B7-H4 peptide, an HVEM peptide, a BTLA peptide, a GAL9 peptide, a TIM3 peptide, a LAG3 peptide, or a KIR polypeptide.

8. The method of any one of claims 1-7, wherein the first immune checkpoint polypeptide is an IDO1 polypeptide and wherein the second immune checkpoint polypeptide is a PD-L1 polypeptide.

9. The method of claim 8, wherein the IDO1 polypeptide consists of up to 50 consecutive amino acids of SEQ ID NO: 1, and wherein said consecutive amino acids comprise the sequence of ALLEIASCL (SEQ ID NO: 2) or the sequence of DTLLKALLEIASCLEKALQVF (SEQ ID NO: 3).

10. The method of claim 8 or 9, wherein the IDO1 polypeptide comprises or consists of the sequence of ALLEIASCL (SEQ ID NO: 2) or the sequence of DTLLKALLEIASCLEKALQVF (SEQ ID NO: 3).

11. The method of claim 8, wherein the PD-L1 polypeptide consists of up to 50 consecutive amino acids of SEQ ID NO: 14, and wherein said consecutive amino acids comprise the sequence of any one of SEQ ID NOs: 15 to 32.

12. The method of claim 8, wherein the PD-L1 polypeptide consists of up to 50 consecutive amino acids of SEQ ID NO: 14, and wherein said consecutive amino acids comprise the sequence of any one of SEQ ID NOs: 15, 25, 28 or 32.

13. The method of claim 8, wherein the PD-L1 peptide comprises or consists of the sequence of FMTYWHLLNAFTVTVPKDL (SEQ ID NO: 32).

14. The method of claim 8, wherein the IDO1 polypeptide comprises or consists of the sequence of ALLEIASCL (SEQ ID NO: 2) or the sequence of DTLLKALLEIASCLEKALQVF (SEQ ID NO: 3) and the PD-L1 peptide comprises or consists of the sequence of FMTYWHLLNAFTVTVPKDL (SEQ ID NO: 32).

15. The method of any one of claims 1-14, wherein the immune checkpoint inhibitor is an antibody or small molecule inhibitor (SMI).

16. The method of claim 15, wherein the SMI is an inhibitor of IDO1.

17. The method of claim 16, wherein the SMI is selected from Epacadostat (INCB24360), Indoximod, GDC-0919 (NLG919), and F001287.

18. The method of claim 15, wherein the antibody binds to CTLA4 or PD1.

19. The method of claim 18, wherein the antibody that binds to CTLA4 is ipilimumab.

20. The method of claim 18, wherein the antibody that binds to PD-1 is pembrolizumab or nivolumab.

21. The method of any one of claims 1-20, wherein (a) and (b) are administered as a first composition and (c) is administered as a second composition.

22. The method of any one of claims 1-20, wherein (a), (b), and (c) are administered as one composition.

23. The method of claim 21 or 22, wherein the compositions further comprise an adjuvant or carrier.

24. The method of claim 23, wherein the adjuvant is selected from herein said adjuvant is a Montanide ISA adjuvant, a bacterial DNA adjuvant, an oil/surfactant adjuvant, a viral dsRNA adjuvant, an imidazoquinoline, and GM-CSF.

25. The method of claim 24, wherein the Montanide ISA adjuvant is selected from Montanide ISA 51 and Montanide ISA 720.

26. The method of any one of claims 3-25, wherein the disease does not progress for at least 12 months, at least 2 years, at least 3 years, at least 4 years, at least 5 years, or longer after completion of treatment.

27. The method of any one of claims 1-26, wherein the cancer is selected from prostate cancer, brain cancer, breast cancer, colorectal cancer, pancreatic cancer, ovarian cancer, lung cancer, cervical cancer, liver cancer, head/neck/throat cancer, skin cancer, bladder cancer, or a hematologic cancer.

28. The method of any one of claims 1-26, wherein the cancer is a solid tumor cancer selected from an adenoma, an adenocarcinoma, a blastoma, a carcinoma, a desmoid tumour, a desmopolastic small round cell tumour, an endocrine tumour, a germ cell tumour, a lymphoma, a leukaemia, a sarcoma, a Wilms tumour, a lung tumour, a colon tumour, a lymph tumour, a breast tumour, or a melanoma.

29. The method of any one of claims 1-26, wherein the cancer is metastatic melanoma.

30. The method of any one of claims 1-29, wherein the subject has an immune profile indicative of response to treatment with the first immune checkpoint polypeptide or the polynucleotide encoding the same, the second immune checkpoint polypeptide or the polynucleotide encoding the same, and the immune checkpoint inhibitor.

31. A method for treating a cancer in a subject in need thereof comprising administering to the subject:
a) an IDO immune checkpoint polypeptide or a polynucleotide encoding the same, wherein the IDO polypeptide comprises or consists of the sequence of ALLEIASCL (SEQ ID NO: 2) or the sequence of
(SEQ ID NO: 3)
DTLLKALLEIASCLEKALQVF;
b) a PD-L1immune checkpoint polypeptide or a polynucleotide encoding the same, wherein the PD-L1 polypeptide comprises or consists of the sequence of FMTYWHLLNAFTVTVPKDL (SEQ ID NO: 32); and
c) an immune checkpoint inhibitor, wherein the immune checkpoint inhibitor is an anti-PD1 antibody.

32. A method of preventing disease progression in a subject suffering from a cancer comprising administering to the subject:
a) an IDO immune checkpoint polypeptide or a polynucleotide encoding the same, wherein the IDO polypeptide comprises or consists of the sequence of ALLEIASCL (SEQ ID NO: 2) or the sequence of
(SEQ ID NO: 3)
DTLLKALLEIASCLEKALQVF;
b) a PD-L1immune checkpoint polypeptide or a polynucleotide encoding the same, wherein the PD-L1 polypeptide comprises or consists of the sequence of FMTYWHLLNAFTVTVPKDL (SEQ ID NO: 32); and
c) an immune checkpoint inhibitor, wherein the immune checkpoint inhibitor is an anti-PD1 antibody.

33. A method of reducing tumor volume in a subject suffering from a cancer comprising administering to the subject:
a) an IDO immune checkpoint polypeptide or a polynucleotide encoding the same, wherein the IDO polypeptide comprises or consists of the sequence of ALLEIASCL (SEQ ID NO: 2) or the sequence of
(SEQ ID NO: 3)
DTLLKALLEIASCLEKALQVF;
b) a PD-L1 immune checkpoint polypeptide or a polynucleotide encoding the same, wherein the PD-L1 polypeptide comprises or consists of the sequence of FMTYWHLLNAFTVTVPKDL (SEQ ID NO: 32); and
c) an immune checkpoint inhibitor, wherein the immune checkpoint inhibitor is an anti-PD1 antibody.

34. The method of any one of claims 30-33, wherein the subject has an immune profile indicative of response to treatment with the IDO polypeptide or polynucleotide encoding the same, the PD-L1 polypeptide or polynucleotide encoding the same, and the anti-PD1 antibody.

35. A kit comprising:
a) a first immune checkpoint polypeptide or a polynucleotide encoding the same;
b) a second immune checkpoint polypeptide or a polynucleotide encoding the same; and
c) an immune checkpoint inhibitor.

36. The kit of claim 35, wherein (a) and (b) are provided as a single composition, in a separate sealed container from (c).

37. An immunotherapeutic composition for use in a method for the prevention or treatment of cancer in a subject, wherein the immunotherapeutic composition comprises (a) and/or (b) as defined in claim 1, and wherein the method is as defined in claim 1.

38. Use of an immunotherapeutic composition in the manufacture of a medicament for the prevention or treatment of cancer in a subject, the immunotherapeutic composition comprising (a) and/or (b) as defined in claim 1, which is formulated for administration before, concurrently with, and/or after an immune checkpoint inhibitor.

39. An immunotherapeutic composition for use according to claim 37, or the use according to claim 38, wherein the subject has an immune profile indicative of response to treatment with the first immune checkpoint polypeptide or the polynucleotide encoding the same, the second immune checkpoint polypeptide or the polynucleotide encoding the same, and the immune checkpoint inhibitor.

40. The immunotherapeutic composition for use according to claim 39 or the use according to claim 39, wherein the first immune polypeptide is an IDO1 polypeptide, the second immune polypeptide is a PD-L1 polypeptide and the immune checkpoint inhibitor is an antibody that binds to PD1.

41. The method of claim 30 or 34, or the immunotherapeutic composition for use according to claim 39 or 40, or the use according to claim 39 or 40, wherein the immune profile comprises one or more of the following:
a) a decrease in CD4+ T regulatory cells compared to a control subject group;
b) a decrease in CD28⁺CD4⁺ T cells compared to a control subject group;
c) an increase in LAG-3+CD4+ T cells compared to a control subject group;
d) a decrease in monocytic-myeloid derived suppressor cells (mMDSCs) compared to a control subject group;
e) an increase in CD56^{dim}CD16+ Natural Killer (NK) cells compared to a control subject group;
f) a decrease in CD56^{bright}CD16− NK cells compared to a control subject group; and/or
g) an increase in conventional dendritic cells type 2 (cDC2) compared to a control subject group.

42. The method, the immunotherapeutic composition, or the use of claim 41, wherein the cell populations are determined by FACS analysis of a peripheral blood sample obtained from the subject.

43. A method for stratifying a cancer patient into one of at least two treatment groups, wherein said method comprises analysing one or more cell populations in a peripheral blood sample from the patient to determine an immune profile; and:
i. stratifying the patient into a first treatment group if the immune profile determined is indicative of response to treatment with an IDO1 polypeptide, a PD-L1 polypeptide and an antibody that binds to PD1; or
ii. stratifying the patient into a second treatment group if the immune profile determined in step indicates that the subject will not respond to said treatment.

44. The method of claim 43, wherein the first treatment group is to be treated, or is treated with, the IDO1 polypeptide, the PD-L1 polypeptide and the antibody that binds to PD1 and the second treatment group is to be treated with, or is treated with, one or more alternative therapies.

45. The method any one of claims 39-44, wherein the immune profile is a baseline immune profile.

46. The method of any one of claims 43-45, wherein an immune profile indicative of response to treatment comprises or more of:
a) a decrease in CD4⁺ T regulatory cells compared to a control subject population;
b) a decrease in CD28⁺CD4⁺ T cells compared to a control subject population;
c) an increase in LAG-3⁺ CD4⁺ T cells compared to a control subject population;
d) a decrease in monocytic-myeloid derived suppressor cells (mMDSCs), compared to a control subject population;
e) an increase in CD56^{dim}CD16+ Natural Killer (NK) cells compared to a control subject population;
f) a decrease in CD56^{bright}CD16− Natural Killer (NK) cells compared to a control subject population; and
g) an increase in conventional dendritic cells type 2 (cDC2) compared to a control subject population.

47. A method of monitoring the response of a cancer patient to treatment with an IDO1 polypeptide, a PD-L1 polypeptide and an antibody that binds to PD1, wherein said method comprises analysing one or more cell populations in a peripheral blood sample from the patient to determine an immune profile, and:
i. determining that the patient is responding to treatment if the patient has an immune profile indicative of response to treatment; or
ii. determining that the patient is not responding to treatment if the patient does not have an immune profile indicative of response to treatment.

48. The method of claim 47, wherein the immune profile indicative of response to treatment comprises increased expression of CD28, HLA-DR, CD39, TIGIT and/or TIMN-3 on CD4+ T cells and/or increased expression of HLA-DR, CD39, LAG-3 and/or TIGIT on CD8+ T-cells.

49. The method of any one of claims 40, 43, 44 or 47, wherein the IDO1 polypeptide is as defined in claim 9 or 10 and/or the PD-L1 polypeptide is as defined in any one of claims 11-13 and/or the antibody that binds to PD1 is pembrolizumab or nivolumab.

50. The method of any one of claims 31-49, wherein the cancer patient has a metastatic melanoma.
